SEQUENCE LISTING (11 sequences)

SEQ ID NO: 1 - protein, 12 aa, Artificial Sequence
  DTC-stapled PMI peptide
  MOD_RES (1)..(1): ACETYLATION
  MISC_FEATURE (1)..(5): K stapled to C with dithiocarbamate moiety
KSFACYWNLLSP

SEQ ID NO: 2 - protein, 12 aa, Artificial Sequence
  DTC-stapled PMI peptide
  MOD_RES (1)..(1): ACETYLATION
  MISC_FEATURE (1)..(1): C stapled to K with dithiocarbamate moiety
CSFAKYWNLLSP

SEQ ID NO: 3 - protein, 12 aa, Artificial Sequence
  DTC-stapled PMI peptide
  MOD_RES (1)..(1): ACETYLATION
  MISC_FEATURE (2)..(6): K stapled to C with dithiocarbamate moiety
TKFAECWNLLSP

SEQ ID NO: 4 - protein, 12 aa, Artificial Sequence
  DTC-stapled PMI peptide
  MOD_RES (1)..(1): ACETYLATION
  MISC_FEATURE (2)..(6): C stapled to K with dithiocarbamate moiety
TCFAEKWNLLSP

SEQ ID NO: 5 - protein, 12 aa, Artificial Sequence
  DTC-stapled PMI peptide
  MOD_RES (1)..(1): ACETYLATION
  MISC_FEATURE (4)..(8): K stapled to C with dithiocarbamate moiety
TSFKEYWCLLSP

SEQ ID NO: 6 - protein, 12 aa, Artificial Sequence
  DTC-stapled PMI peptide
  MOD_RES (1)..(1): ACETYLATION
  MISC_FEATURE (4)..(8): C stapled to K with dithiocarbamate moiety
TSFCEYWKLLSP

SEQ ID NO: 7 - protein, 12 aa, Artificial Sequence
  DTC-stapled PMI peptide
  MOD_RES (1)..(1): ACETYLATION
  MISC_FEATURE (5)..(9): K stapled to C with dithiocarbamate moiety
TSFAKYWNCLSP

SEQ ID NO: 8 - protein, 12 aa, Artificial Sequence
  DTC-stapled PMI peptide
  MOD_RES (1)..(1): ACETYLATION
  MISC_FEATURE (5)..(9): C stapled to K with dithiocarbamate moiety
TSFACYWNKLSP

SEQ ID NO: 9 - protein, 12 aa, Artificial Sequence
  DTC-stapled PMI peptide
  MOD_RES (1)..(1): ACETYLATION
  MISC_FEATURE (8)..(12): K stapled to C with dithiocarbamate moiety
TSFAEYWKLLSC

SEQ ID NO: 10 - protein, 12 aa, Artificial Sequence
  DTC-stapled PMI peptide
  MOD_RES (1)..(1): ACETYLATION
  MISC_FEATURE (8)..(12): C stapled to K with dithiocarbamate moiety
TSFAEYWCLLSK

SEQ ID NO: 11 - protein, 12 aa, Artificial Sequence
  DTC-stapled p53 peptide
  MOD_RES (1)..(1): ACETYLATION
  MISC_FEATURE (5)..(9): C stapled to K with dithiocarbamate moiety
QETFCDLWKLLP

(19) United States
(12) Patent Application Publication
(10) Pub. No.: US 2022/0089638 A1
(43) Pub. Date: Mar. 24, 2022

(54) DITHIOCARBAMATE STAPLED PEPTIDES AND METHODS OF MAKING AND USE THEREOF

(71) Applicant: UNIVERSITY OF MARYLAND, BALTIMORE, BALTIMORE, MD (US)

(72) Inventors: Wuyuan LU, Shanghai (CN); Xiang LI, Shanghai (CN)

(21) Appl. No.: 17/078,016

(22) Filed: Oct. 22, 2020

Related U.S. Application Data
(60) Provisional application No. 62/924,300, filed on Oct. 22, 2019.

(51) Int. Cl. C07K 1/107; C07K 7/08

(52) U.S. Cl. CPC C07K 1/1077; C07K 7/08

(57) ABSTRACT

Dithiocarbamate stapled peptides and methods of making and using the same for treating a condition associated with p53, such as cancer, are provided.

### CROSS-REFERENCE TO RELATED APPLICATIONS

This application claims the benefit of priority of U.S. Provisional Application No. 62/924,300, filed Oct. 22, 2019, which is incorporated by reference herein in its entirety.

### STATEMENT REGARDING FEDERALLY SPONSORED RESEARCH OR DEVELOPMENT

This invention was made with government support under Grant Number CA 167296 awarded by the National Institutes of Health. The government has certain rights in the invention.

### REFERENCE TO SEQUENCE LISTING SUBMITTED ELECTRONICALLY

The sequence listing contained in the file named “115834-5023-US_ST25.txt”, created on Oct. 22, 2020, and having a size of 4.43 kilobytes, has been submitted electronically herewith via EFS-Web, and the contents of the txt file are hereby incorporated by reference in their entirety.

### FIELD

The disclosure relates generally to dithiocarbamate stapled peptides and methods of making using the same for treating conditions associated with p53, including cancer.

### BACKGROUND

Peptides are effective inhibitors of protein-protein interactions (PPI) and superior in many aspects as therapeutics to small molecule and protein drugs. However, peptides have two major pharmacological disadvantages—strong susceptibility to proteolytic degradation in vivo and poor membrane permeability, severely limiting their therapeutic efficacy. For small peptides that adopt an α-helical structure upon interaction with target protein, various side chain stapling chemistries have been developed to improve their pharmacological properties via a pre-formed stable α-helix, among which the elaborate “hydrocarbon stapling” technique is probably best known.

Despite its success in peptide drug design, hydrocarbon stapling can be technically cumbersome and costly due to the use of conformationally constrained unnatural amino acids; all-hydrocarbon crosslinks also invariably decrease peptide solubility, potentially limiting drug concentration in vivo.

### SUMMARY

The disclosure provides a method for preparing a dithiocarbamate stapled peptide. The method includes contacting a peptide comprising a dehydroalanine residue and a lysine residue with carbon disulfide to form a dithiocarbamate linker.

In some embodiments, the method further includes contacting a peptide comprising a cysteine residue and a lysine residue with a reagent to convert the cysteine residue into a dehydroalanine residue. In some embodiments, the cysteine residue and the lysine residue are separated by one or more amino acid residues. In some embodiments, the reagent comprises a 1,4-dihalobutane group. In some embodiments, the reagent is selected from 2,5-dibromohexanediamide, 1,4-butanediol dimethanesulfonate, 1,4-dibromobutane, 1,4-diiodobutane, and methyl 2,5-dibromopentanoate. In some embodiments, the reagent is selected from O-mesitylenesulfonylhydroxylamine, 5,5-dithio-bis-(2-nitrobenzoic acid (Ellman's reagent), and 1,2-bis(bromomethyl)benzene. In some embodiments, the method further includes adding the reagent to a buffer solution. In some embodiments, the buffer solution is at a pH in a range of about 8 to about 9. In some embodiments, the method further includes adding the peptide comprising a cysteine residue and a lysine residue to a buffer solution. In some embodiments, the buffer solution is at a pH in a range of about 2 to about 3. In some embodiments, the buffer solution comprises guanidine hydrochloride and sodium hydrogen phosphate (Na₂HPO₄). In some embodiments, the method further comprises adding the buffer solution comprising the peptide comprising a cysteine residue and a lysine residue to the buffer solution comprising the reagent. In some embodiments, the method further comprises adding the carbon disulfide to a solution comprising one or more alcohols and the peptide comprising a dehydroalanine residue and a lysine residue. In some embodiments, the alcohol is selected from methanol, ethanol, n-propanol, isopropanol, n-butanol, s-butanol, and t-butanol. In some embodiments, the alcohol is ethanol. In some embodiments, the method further includes adding a base to the solution of the peptide comprising a dehydroalanine residue and a lysine residue. In some embodiments, base is triethylamine. In some embodiments, the dithiocarbamate stapled peptide comprises a peptide backbone comprising 3 or more amino acid residues. In some embodiments, the peptide backbone comprises 3 to 20 amino acid residues. In some embodiments, the peptide backbone comprises 12 amino acids. In some embodiments, the dehydroalanine residue and the lysine residue are separated by 1 to 8 amino acid residues. In some embodiments, the dehydroalanine residue and the lysine residue are separated by 3 amino acid residues.

The disclosure also provides a stapled peptide comprising a peptide backbone and a staple, wherein the peptide backbone comprises three or more amino acid residues, the staple comprises a dithiocarbamate moiety and is attached to a cysteine residue and a lysine residue, and the cysteine and lysine residues are separated by one or more amino acid residues.

In some embodiments, the peptide backbone comprises 3 to 20 amino acids. In some embodiments, the peptide backbone comprises 12 amino acids. In some embodiments, the cysteine residue and the lysine residue are separated by 1 to 8 amino acid residues. In some embodiments, the cysteine residue and the lysine residue are separated by 3 amino acid residues. In some embodiments, the stapled peptide is capped at the 5′ end, at the 3′ end, or at both ends. In some embodiments, the stapled peptide is capped at the 5′ end. In some embodiments, the stapled peptide is capped with one or more of a group selected from formyl, acetyl, propanoyl, hexanoyl, and myristoyl. In some embodiments, the stapled peptide is capped with an acetyl group. In some embodiments, the stapled peptide comprises one staple. In some embodiments, the stapled peptide comprises two or more staples, wherein the staples are chemically similar or chemically different. In some embodiments, the staple comprises a structure of formula (I). In some embodiments, the stapled peptide is selected from SEQ ID NO: 1 to SEQ ID NO: 11. In some embodiments, the peptide is selected from SEQ ID NO: 5 and SEQ ID NO: 9:

The disclosure also provides a pharmaceutical composition for treating a condition alleviated by inducing p53 activity. In some embodiments, the pharmaceutical composition comprising one or more stapled peptides according to the disclosure, or a pharmaceutically acceptable salt, solvate, hydrate, cocrystal, or prodrug thereof, and a pharmaceutically acceptable carrier. In some embodiments, the condition is cancer.

The disclosure also provides a pharmaceutical composition for treating cancer. In some embodiments, the pharmaceutical composition comprising one or more stapled peptides of the disclosure, or a pharmaceutically acceptable salt, solvate, hydrate, cocrystal, or prodrug thereof, and a pharmaceutically acceptable carrier. In some embodiments, the cancer is selected from bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thymoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophageal tumors, hematological neoplasms, non-small-cell lung cancer, chronic myelocytic leukemia, diffuse large B-cell lymphoma, esophagus tumor, follicle center lymphoma, head and neck tumor, hepatitis C virus induced cancer, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

The disclosure also provides a method of treating a condition by inducing p53 activity in a patient in need of said treatment. In some embodiments, the method cincludes administering to the patient a therapeutically effective amount of a stapled peptide of the disclosure, or a pharmaceutically acceptable salt, solvate, hydrate, cocrystal, or prodrug thereof. In some embodiments, the condition is cancer.

The disclosure also provides a method of treating or preventing cancer. In some embodiments, the method comprising administering to a patient a therapeutically effective amount of a stapled peptide of the disclosure, or a pharmaceutically acceptable salt, solvate, hydrate, cocrystal, or prodrug thereof. In some embodiments, the cancer is selected from bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thymoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophageal tumors, hematological neoplasms, non-small-cell lung cancer, chronic myelocytic leukemia, diffuse large B-cell lymphoma, esophagus tumor, follicle center lymphoma, head and neck tumor, hepatitis C virus induced cancer, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

### BRIEF DESCRIPTION OF THE FIGURES

FIG. 1A-FIG. 1J: FIG. 1A illustrates the schematic representation of the DTC chemistry linking the side chains of Lys and Cys at (i, i+4) positions. FIG. 1B and FIG. 1C illustrate SPR-based equilibrium competition binding assays for peptides interacting with MDM2 and MDMX. FIG. 1D illustrates co-crystal structure of PMI(8,12)-a (green) or PMI (cyan) in complex with MDM2 (yellow). FIG. 1E illustrates co-crystal structure of PMI(4,8)-a (yellow) or PMI (cyan) in complex with MDMX (gray). FIG. 1F illustrates amino acid sequence and chemical structure of ^{DTC}PMI. FIG. 1G illustrates dose-dependent anti-proliferative activity of ^{DTC}PMI against isogenic HCT116p53^{+/+} and p53^{-/-} cell lines. FIG. 1H illustrates Western blot analysis of the expression of MDM2, p21 and p53 in HCT116p53^{+/+} cells treated with ^{DTC}PMI. FIG. 1I and FIG. 1J illustrate ^{DTC}PMI-induced apoptosis of HCT116p5.3^{+/+} cells as analyzed by flow cytometry.

FIG. 2 illustrates the synthetic route of DTC-stapled PMI(1,5)-a.

FIG. 3 illustrates the structures of DTC-stapled PMI peptides of the disclosure.

FIG. 4 illustrates representative HPLC chromatograms of purified PMI(4,8)-a and PMI(8,12)-a.

FIG. 5A-FIG. 5B illustrates circular dichroism spectra of linear PMI-0 and DTC-stapled peptides.

FIG. 6 illustrates the superposition of MDM2-PMI(8,12)-a and MDMX-PMI(4,8)-a copies within the asymmetric unit of each crystal form. The root mean square deviation (RMSD) between the 12 copies of MDM2-PMI(8,12)-a complex (left) in the crystal ranges from 0.479-1.348 Å, 0.393-1.180 Å for MDM2 alone and 0.286-1.476 Å for the peptides (Table S4). The RMSD between the 8 MDMX-PMI(4, 8)-1 complexes (right) ranges from 0.498-0.976 Å in the crystal, 0.368-0.774 Å for MDMX and 0.274-2.188 Å for PMI(4, 8)-1 (Table 5).

FIG. 7 illustrates MDM2-PMI(8,12)-a and MDMX-PMI(4,8)-a complex interfaces. The MDM2-PMI(8,12)-a, MDM2-PMI (PDB code: 3EQS), MDMX-PMI(4,8)-a and MDMX-PMI (PDB code: 3EQY) complex structures were superimposed based on MDM2 (top) and MDMX (bottom). The PMI peptides are shown as ribbon-ball-stick representations. For clarity only side chains of residues of MDM2 and MDMX forming the interface involved in hydrogen bonds and hydrophobic contacts are shown as ball-sticks and residues which differ between the stapled PMI and PMI complexes are colored in red. The same set of residues with the exception of K⁵¹ and Met¹⁰² that lines the PMI binding pocket within the MDM2 molecule is involved in PMI(8,12)-a peptide binding (residues 54-55, 57-58, 61-62, 67, 72-73, 75, 86, 91, 93-94, 96, 99-100 of MDM2). In addition, PMI(8,12)-a makes one new hydrophobic contact to I¹⁰³ of MDM2. There are also three direct protein-peptide H-bounds formed at the MDM2-PMI(8,12)-a contact interface (Q⁷² Oε1 to F³N, L⁵⁴ O to W³ Nε1, Y¹⁰⁰ (OH) to L¹⁰ O) with elongated H-bond of Q⁷² Oε¹ to F³ N. Residues 53-54, 56-57, 60-61, 66, 71-72, 74, 90, 92-93, 95, 98-99 of MDMX line the PMI(4,8)-a binding pocket. The PMI(4,8)-a binding doesn't involve V⁴⁹ and L¹⁰² of MDMX which are engaged in PMI binding. A new contact to K⁵⁰ of MDMX is formed to accommodate M¹¹ of PMI(4,8)-a. There are also two direct protein-peptide H-bounds formed at the MDMX-PMI(4,8)-a contact interface (Q71 Oε1 to F3 N, M⁵³ O to W³ Nε1 and Y⁹⁹ (OH) to S¹¹ O) with elongated H-bond between Q71 Oε1 to F3 N and Y99 (OH) to S¹¹ O).

FIG. 8 illustrates the superposition of PMI(8,12)-a and PMI(4,8)-a peptides from the crystal asymmetric unit to each other and to the parent PMI peptide. PMI(8,12)-a and PMI(4,8)-a peptides could be superimposed with an average RMSD value of 0.946 Å and 0.943 Å for the main chain atoms of 11 residues (Thr¹-Ser¹¹) among themselves and with PMI peptides, respectively.

FIG. 9 illustrates the structural analysis of interactions of stapled PMI with MDM2 and MDMX. Analysis of the peptide binding interface. The relative contribution of each residue of PMI(8,12)-a and PMI(4,8)-a (green/yellow) and PMI (cyan) to MDM2/MDMX interface is shown as the buried surface area (BSA, top panel) and the solvation energy in kcal/mol (ΔiG, bottom panel) of each position as calculated by PISA. BSA represents the solvent-accessible surface area of the corresponding residue that is buried upon interface formation and the solvation energy gain of the interface is calculated as the difference in solvation energy of a residue between the dissociated and associated structures. A positive solvation energy corresponds to a negative contribution to the solvation energy gain of the interface or put another way, the hydrophobic effect. Hydrogen bonds and salt bridges are not included in ΔiG. When more than one copy of the peptide is present in the asymmetric unit values are shown as the mean with the range displayed as an error bar. The sequence for each position is shown on the bottom. E⁵ of PMI peptides is not shown since it is not contributing to the binding in any of complex shown.

FIG. 10 illustrates the stability of PMI-0 and/or PMI(8,12)-a in the presence of cathepsin G or GSH as monitored by HPLC.

FIG. 11 illustrates experimental data demonstrating the viability of HCT116 p53^{+/+} and HCT116 p53^{-/-} cell lines in the presence of PMI-0 and stapled PMI peptides.

FIG. 12 illustrates binding curves of representative PMI peptides with MDM2 (left) and MDMX (right) as determined by fluorescence polarization.

FIG. 13 illustrates experimental data demonstrating the viability of HCT116 p53^{+/+} and HCT116 p53^{-/-} cell lines in the presence of linear ^{DTC}PMI control.

FIG. 14 illustrates fitted curves of viability of HCT116 p53^{+/+} and HCT116 p53^{-/-} cell lines in the presence of ^{DTC}PMI.

FIG. 15 illustrates experimental data demonstrating ^{DTC}PMI-induced apoptosis of HCT 116 p53^{+/+} cells as measured by flow cytometry

FIG. 16 illustrates one embodiment of the synthetic route of DTC-stapled PMI(1,5)-a.

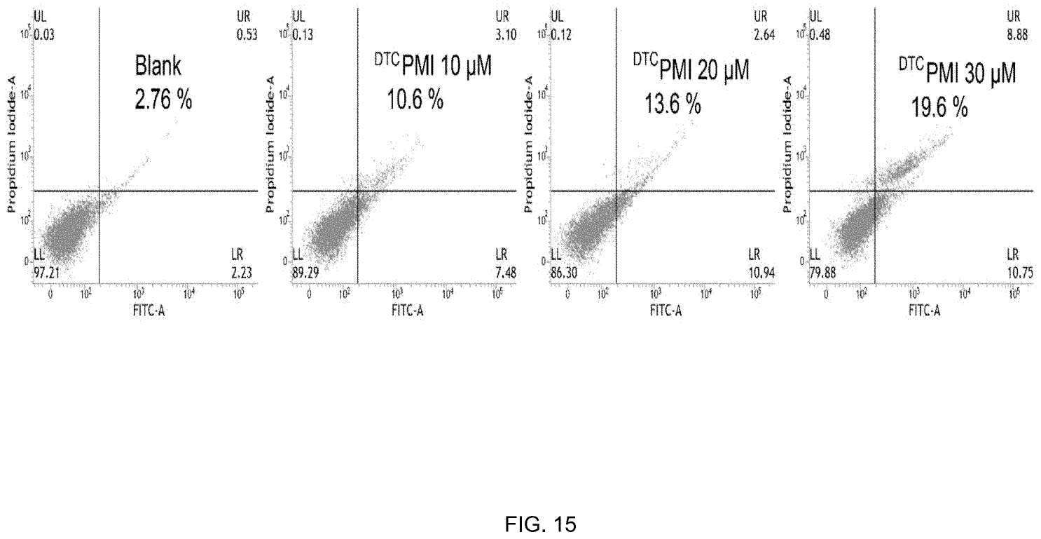

FIG. 17 illustrates a schematic of the dithiocarbamate stapling of PMI, followed by traversing the cancer cell and activating p53 to induce apoptosis.

FIG. 18A-FIG. 18C illustrate examples of DTC stapling chemistry. FIG. 18A illustrates a schematic representation of DTC chemistry linking the side chains of Lys and Cys at (i, i+4) positions. FIG. 18B illustrates structures of examples of DTC-stapled PMI peptides. FIG. 18C illustrates formation of the DTC staple as one predominant product from the PMI-derived peptide Ac-TSFAEKWCLLSK-NH₂ according to HPLC analytic traces.

FIG. 19A-FIG. 19C illustrate HPLC chromatograms and MS spectra of DTC-stapled peptides. Bottom panel: PMI-0, PMI(4,8)-a and PMI(8,12)-a analyzed by HPLC at different gradients, 30-60% B (FIG. 19B) and 35-45% B (FIG. 19C) over 30 min (B=acetonitrile).

FIG. 20A-FIG. 20E: FIG. 20A illustrates the structure of ^{DTC}p53 and HPLC and MS chromatograms. of ^{DTC}p53. FIG. 20B-FIG. 20E illustrate binding curves with MDM2 (FIG. 20B and FIG. 20D) and MDMX (FIG. 20C and FIG. 20E) as determined by SPR and FP.

FIG. 21 illustrates experimental data demonstrating that tryptic digestion coupled with mass spectrometry analysis confirmed the DTC staple formed by Cys and Lys at (i, i+4) positions. Note: the 812.5 Da mass peak is the sodium adduct of the DTC-stapled peptide fragment (790.5 Da).

FIG. 22A-FIG. 22E illustrate the characterization of representative DTC-stapled PMI peptides. FIG. 22A illustrates MDM2 at 25 or 50 nM and FIG. 22B illustrates MDMX at 100 nM with PMI-0, PMI(4,8)-a and PMI(8,12)-a as quantified by SPR-based competitive binding assays. FIG. 22C and FIG. 22D illustrate MDM2 (FIG. 22C) and MDMX (FIG. 22D) at 50 nM with PMI-0, PMI(4,8)-a and PMI(8,12)-a as quantified by FP-based competitive binding assays. Kd and Ki values were obtained through a non-linear regression analysis, and each curve is the mean of three independent measurements. Two replicates and three independent experiments were performed. FIG. 22E illustrates Circular dichroism spectra of PMI-0, PMI(4,8)-a and PMI(8,12)-a. The experiment was repeated independently twice with similar results.

FIG. 23 illustrates circular dichroism spectra of DTC-stapled PMIs.

FIG. 24A-FIG. 24F illustrate the structural validation of DTC staples. FIG. 24A illustrates the co-crystal structure of PMI(8,12)-a (green) or PMI (cyan) in complex with MDM2 (yellow). FIG. 24B illustrates the co-crystal structure of PMI(4,8)-a (yellow) or PMI (cyan) in complex with MDMX (gray). FIG. 24C and FIG. 24D illustrate the superposition of PMI(8,12)-a and PMI(4,8)-a peptides from the crystal asymmetric unit to each other and to the parent PMI peptide. PMI(8,12)-a and PMI(4,8)-a peptides could be superimposed with an average RMSD value of 0.946 Å and 0.943 Å for the main chain atoms of 11 residues (Thr¹-Ser¹¹) among themselves and with PMI peptides, respectively. FIG. 24E and FIG. 24F illustrate the electron density maps of the DTC staples seen in PMI(8,12)-a (left) and PMI(4,8)-a (right) contoured at 1.0 σ level. D-cysteine in black is modeled at the same position, where no electron density was observed.

FIG. 25 illustrates the superposition of MDM2-PMI(8,12)-a and MDMX-PMI(4,8)-a copies within the asymmetric unit of each crystal form. The root mean square deviation (RMSD) between the 12 copies of MDM2-PMI(8,12)-a complex (left) in the crystal ranges from 0.479-1.348 ∈, 0.393-1.180 ∈ for MDM2 alone and 0.286-1.476 ∈ for the peptides (Table 8). The RMSD between the 8 MDMX-PMI(4, 8)-1 complexes (right) ranges from 0.498-0.976 Å in the crystal, 0.368-0.774 Å for MDMX and 0.274-2.188 Å for PMI(4, 8)-1 (Table 9).

FIG. 26 illustrates MDM2-PMI(8,12)-a and MDMX-PMI(4,8)-a complex interfaces. The MDM2-PMI(8,12)-a, MDM2-PMI (PDB code: 3EQS), MDMX-PMI(4,8)-a and MDMX-PMI (PDB code: 3EQY) complex structures were superimposed based on MDM2 (top) and MDMX (bottom). The PMI peptides are shown as ribbon-ball-stick representations. For clarity only side chains of residues of MDM2 and MDMX forming the interface involved in hydrogen bonds and hydrophobic contacts are shown as ball-sticks and residues which differ between the stapled PMI and PMI complexes are colored in red. The same set of residues with the exception of K⁵¹ and Met¹⁰² that lines the PMI binding pocket within the MDM2 molecule is involved in PMI(8,12)-a peptide binding (residues 54-55, 57-58, 61-62, 67, 72-73, 75, 86, 91, 93-94, 96, 99-100 of MDM2). In addition, PMI(8,12)-a makes one new hydrophobic contact to I¹⁰³ of MDM2. There are also three direct protein-peptide H-bounds formed at the MDM2-PMI(8,12)-a contact interface (Q⁷² Oε1 to F³ N, L⁵⁴ O to W³ Nε1, Y¹⁰⁰ (OH) to L¹⁰ O) with elongated H-bond of Q⁷² Oε¹ to F³ N. Residues 53-54, 56-57, 60-61, 66, 71-72, 74, 90, 92-93, 95, 98-99 of MDMX line the PMI(4,8)-a binding pocket. The PMI(4,8)-a binding does not involve V⁴⁹ and L¹⁰² of MDMX which are engaged in PMI binding. A new contact to K⁵⁰ of MDMX is formed to accommodate M¹¹ of PMI(4,8)-a. There are also two direct protein-peptide H-bounds formed at the MDMX-PMI(4,8)-a contact interface (Q71 Oε1 to F3 N, M⁵³ O to W³ Nε1 and Y⁹⁹ (OH) to S¹¹ O) with elongated H-bond between Q71 Oε1 to F3 N and Y99 (OH) to S¹¹ O).

FIG. 27 illustrates structural analysis of interactions of stapled PMI with MDM2 and MDMX. Analysis of the peptide binding interface. The relative contribution of each residue of PMI(8,12)-a and PMI(4,8)-a (green/yellow) and PMI (cyan) to MDM2/MDMX interface is shown as the buried surface area (BSA, top panel) and the solvation energy in kcal/mol (ΔiG, bottom panel) of each position as calculated by PISA. BSA represents the solvent-accessible surface area of the corresponding residue that is buried upon interface formation and the solvation energy gain of the interface is calculated as the difference in solvation energy of a residue between the dissociated and associated structures. A positive solvation energy corresponds to a negative contribution to the solvation energy gain of the interface or put another way, the hydrophobic effect. Hydrogen bonds and salt bridges are not included in ΔiG. When more than one copy of the peptide is present in the asymmetric unit values are shown as the mean with the range displayed as an error bar. The sequence for each position is shown on the bottom. E⁵ of PMI peptides is not shown since it is not contributing to the binding in any of complex shown.

FIG. 28A-FIG. 28B: FIG. 28A illustrates the stability of PMI-0 and PMI(8,12)-a in the presence of cathepsin G or GSH as monitored by HPLC. FIG. 28B illustrates the stability of PMI-0 and PMI(8,12)-a in the presence of human serum.

FIG. 29A-FIG. 29K illustrates design and functional characterization of ^{DTC}PMI. FIG. 29A illusrates the amino acid sequence and chemical structure of ^{DTC}PMI. FIG. 29B illusrates HPLC chromatograms and MS spectra of ^{DTC}PMI. FIG. 29C illusrates MDM2 at 25 or 50 nM and FIG. 29D illusrates MDMX at 100 nM with ^{DTC}PMI Ctrl. and ^{DTC}PMI as quantified by SPR-based competitive binding assays. FIG. 29E and FIG. 29F illustrate MDM2 (FIG. 29E) and MDMX (FIG. 29F) at 50 nM with ^{DTC}PMI Ctrl. and ^{DTC}PMI as quantified by FP-based competitive binding assays. Kd and Ki values were obtained through a non-linear regression analysis, and each curve is the mean of three independent measurements. Two replicates and three independent experiments were performed. FIG. 29G illusrates circular dichroism spectra of ^{DTC}PMI. FIG. 29H illusrates dose-dependent anti-proliferative activity of ^{DTC}PMI against isogenic HCT116 p53^{+/+} and p53^{-/-} cell lines. FIG. 29I illusrates Western blot analysis of the expression of MDM2, p21 and p53 in HCT116 p53^{+/+} cells treated with ^{DTC}PMI. FIG. 29J and FIG. 29K illustrate ^{DTC}PMI-induced apoptosis of HCT116 p5.3^{+/+} cells as analyzed by flow cytometry. The experiment was repeated independently twice with similar results.

FIG. 30 illustrates confocal microscope images of FITC-labeled ^{DTC}PMI Ctrl. and ^{DTC}PMI localization in HCT116 cells. Both of them showed a diffused intracellular localization, demonstrating efficient cellular uptake.

FIG. 31 illustrates experimental data demonstrating the viability of HCT116 p53^{+/+} and HCT116 p53^{-/-} cell lines in the presence of linear ^{DTC}PMI control.

FIG. 32 illustrates fitted curves of the viability of HCT116 p53^{+/+} and HCT116 p53^{-/-} cell lines in the presence of ^{DTC}PMI.

FIG. 33A-FIG. 33B: FIG. 33A illustrates quantitative Western blot analysis of MDM2, p21 and p53 in HCT116 p53^{+/+} cells treated with different concentrations of ^{DTC}PMI. FIG. 33B illustrates original western blot gel for MDM2, p21 and p53. Lane 1 was for blank control and lanes 5-7 were for ^{DTC}PMI. Three additional lanes (lane 2-4) for peptide samples were unrelated to this work.

FIG. 34 illustrates experimental data demonstrating ^{DTC}PMI-induced apoptosis of HCT 116 p53^{-/-} cells as measured by flow cytometry.

FIG. 35 illustrates a comparison of solubility between DTC- and Hydrocarbon-stapled PMI.

### DETAILED DESCRIPTION

Unless defined otherwise, all technical and scientific terms used herein have the same meaning as is commonly understood by one of skill in the art to which this disclosure belongs. All patents and publications referred to herein are incorporated by reference in their entireties.

### Definitions

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The terms “active pharmaceutical ingredient” and “drug” include, but are not limited to, the compounds described herein and, more specifically, a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, and their features and limitations as described herein.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., increased sensitivity to apoptosis). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs disclosed herein, can also be incorporated into the described compositions and methods.

As used herein, the terms “treat,” “treatment,” and/or “treating” may refer to the management of a disease, disorder, or pathological condition, or symptom thereof with the intent to cure, ameliorate, stabilize, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition).

As used herein, the terms “modulate” and “modulation” refer to a change in biological activity for a biological molecule (e.g., a protein, gene, peptide, antibody, and the like), where such change may relate to an increase in biological activity (e.g., increased activity, agonism, activation, expression, upregulation, and/or increased expression) or decrease in biological activity (e.g., decreased activity, antagonism, suppression, deactivation, downregulation, and/or decreased expression) for the biological molecule.

As used herein, the term “prodrug” refers to a derivative of a compound described herein, the pharmacologic action of which results from the conversion by chemical or metabolic processes in vivo to the active compound. Prodrugs include compounds wherein an amino acid residue, or a polypeptide chain of two or more (e.g., two, three or four) amino acid residues is covalently joined through an amide or ester bond to a free amino, hydroxyl or carboxylic acid group of a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11. The amino acid residues include but are not limited to the 20 naturally occurring amino acids commonly designated by one or three letter symbols but also include, for example, 4-hydroxyproline, hydroxylysine, desmosine, isodesmosine, 3-methylhistidine, beta-alanine, gamma-aminobutyric acid, citrulline, homocysteine, homoserine, ornithine and methionine sulfone.

Additional types of prodrugs are also encompassed. For instance, free carboxyl groups can be derivatized as amides or alkyl esters (e.g., methyl esters and acetoxy methyl esters). Prodrug esters as employed herein includes esters and carbonates formed by reacting one or more hydroxyls of compounds of the method of the disclosure with alkyl, alkoxy, or aryl substituted acylating agents employing procedures known to those skilled in the art to generate acetates, pivalates, methylcarbonates, benzoates and the like. As further examples, free hydroxyl groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxyl and amino groups are also included, as are carbonate prodrugs, sulfonate prodrugs, sulfonate esters and sulfate esters of hydroxyl groups. Free amines can also be derivatized to amides, sulfonamides or phosphonamides. All of the stated prodrug moieties may incorporate groups including but not limited to ether, amine and carboxylic acid functionalities. Moreover, any compound that can be converted in vivo to provide the bioactive agent (e.g., a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11) is a prodrug within the scope of the disclosure. Various forms of prodrugs are well known in the art. A comprehensive description of pro drugs and prodrug derivatives are described in: (a) The Practice of Medicinal Chemistry, Camille G. Wermuth et al., (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds., (Harwood Academic Publishers, 1991). In general, prodrugs may be designed to improve the penetration of a drug across biological membranes in order to obtain improved drug absorption, to prolong duration of action of a drug (slow release of the parent drug from a prodrug, decreased first-pass metabolism of the drug), to target the drug action (e.g. organ or tumor-targeting, lymphocyte targeting), to modify or improve aqueous solubility of a drug (e.g., i.v. preparations and eyedrops), to improve topical drug delivery (e.g. dermal and ocular drug delivery), to improve the chemical/enzymatic stability of a drug, or to decrease off-target drug effects, and more generally in order to improve the therapeutic efficacy of the compounds utilized in the disclosure.

The term “amino acid” as used herein refers to a molecule containing both an amino group and a carboxyl group. Amino acids include alpha-amino acids and beta-amino acids, the structures of which are depicted below. In certain forms, an amino acid is an alpha amino acid. Amino acids can be natural or synthetic. Amino acids include, but are not limited to, the twenty standard or canonical amino acids: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). Common non-standard or non-canonical amino acids include, but are not limited to, dehydroalanine, selenocysteine, pyrrolysine, and N-formylmethionine.

For the avoidance of doubt, it is intended herein that particular features (for example integers, characteristics, values, uses, diseases, formulae, compounds or groups) described in conjunction with a particular aspect, embodiment or example of the disclosure are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The disclosure is not restricted to any details of any disclosed embodiments. The disclosure extends to any novel one, or novel combination, of the features disclosed in this specification (including any accompanying claims, abstract and drawings), or to any novel one, or any novel combination, of the steps of any method or process so disclosed.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the disclosure. All embodiments of the disclosure can, in the alternative, be more specifically defined by any of the transitional terms “comprising,” “consisting essentially of,” and “consisting of.”

Methods of Peptide Stapling

Two major pharmacological hurdles severely limit the widespread use of small peptides as therapeutics: poor proteolytic stability and membrane permeability. Various elaborate side chain stapling chemistries have been developed for α-helical peptides to circumvent this problem, with considerable success.

Hydrocarbon stapling chemistry takes advantage of Grubbs catalysts to crosslink on resin, via ruthenium-catalyzed olefin metathesis, two unnatural amino acids bearing olefinic side chains at (i, i+4) or (i, i+7) positions, and has been successfully used to design various peptide inhibitors with improved proteolytic stability, membrane permeability, and biological activity. One notable example is ALRN-6924, a hydrocarbon-stapled peptide antagonist of the oncogenic proteins MDM2 and MDMX that functionally inhibit the tumor suppressor protein p53. ALRN-6924, in phase 2 clinical trials for advanced solid tumors and lymphomas, kills tumor cells harboring wild-type p53 by antagonizing MDM2 and/or MDMX to reactivate the p53 pathway.

In one aspect, described herein is a novel peptide stapling strategy. In some embodiments, the side chains of Lys and Cys are crosslinked at (i, i+4) positions via a thiocarbonyl group to form the dithiocarbamate (DTC) structure —NH—C(═S)—S—. stapling chemistries have been developed for α-helical peptides to circumvent this problem. In some embodiments, the side chains of residues Lys (i) and Cys (i+4) are linked in a dodecameric peptide antagonist, termed PMI, of the p53-inhibitory oncogenic proteins MDM2 and MDMX. In one embodiment, a dithiocarbamate-stapled PMI derivative, DTC PMI, showed a 50-fold stronger binding to MDM2 and MDMX than its linear counterpart. In some embodiments, the present invention describes DTC PMI or derivatives as p53-activating compounds for anticancer therapy.

As described herein, crystallographic studies of peptide-MDM2/MDMX complexes structurally validated the design of the dithiocarbamate staple bridging Lys and Cys at (i, i+4) positions, and in contrast to PMI and its linear derivatives, the DTC PMI peptide actively traversed the cell membrane and killed HCT116 tumor cells in vitro by activating the tumor suppressor protein p53. In one aspect, the facile and cost-effective stapling chemistry disclosed herein demonstrates an important new tool for the development of peptide therapeutics with improved pharmacological properties.

In one aspect, the present disclosure provides a method for preparing a dithiocarbamate stapled peptide. In some embodiments, the method comprises contacting a peptide comprising a dehydroalanine residue and a lysine residue with carbon disulfide to form a dithiocarbamate linker.

In some embodiments, the synthesis of a stapled peptide first involves the selection of a desired sequence and number of amino acids and amino acid analogues. As one of ordinary skill in the art will realize, the number, stereochemistry, and type of amino acid structures (natural or non-natural) selected will depend upon the size of the peptide to be prepared, the ability of the particular amino acids to generate a desired structural motif (e.g., an α-helix), and any particular motifs that are desirable to mimic protein domains that effectively bind to the target or effector biomolecule. In some embodiments, the peptides are helical. In some embodiments, the peptides are non-helical. In some embodiments, the stapled peptide sequence can parallel a sequence or subsequence of a known peptide or protein and improve the stability or other characteristics of an existing α-helix or other amino acid motif(s) therein. In some embodiments, the stapled peptide sequence can be added to a known peptide or protein to add an α-helix or other amino acid motif(s) wherein none existed before. In some embodiments, the active agent is the stapled peptide. In some embodiments, the peptide has two or more staples. In some embodiments, the stapled sequences can be the same or different.

There are various strategies for generating stapled helical peptides. In one embodiment, the method includes the use of cysteine side chains for forming disulfide bridges and thioether formation. Other non-limiting methods involve ring-closing metathesis; biaryl linkage of functionalized synthetic amino acids involving borylated phenylalanine derivatives; or “click chemistry”, whereby cycloaddition between an azide and a terminal or internal alkyne yields a 1,2,3-Triazole. These syntheses are expensive and laborious. The disclosed methods and stapled peptides offer improvements over the foregoing peptide staple technology.

In some embodiments, the method further comprises adding the carbon disulfide to a solution comprising one or more alcohols and the peptide comprising a dehydroalanine residue and a lysine residue. Non-limiting examples of alcohols include methanol, ethanol, n-propanol, isopropanol, n-butanol, s-butanol, and t-butanol. In some embodiments, the alcohol is ethanol.

In some embodiments, the method further comprises adding a base to the solution of the peptide comprising a dehydroalanine residue and a lysine residue. Non-limiting examples of bases include trimethylamine, N,N-diisopropylethylamine (DIEA), potassium t-butoxide, or 1,8-diazabicyclo[5.4.0]undec-7-ene (DBU). In some embodiments, the base is trimethylamine.

In some embodiments, the dehydroalanine residue and the lysine residue are separated by one or more amino acid residues. In some embodiments, the dehydroalanine residue and the lysine residue are separated by 2 or more amino acid residues. In some embodiments, the dehydroalanine residue and the lysine residue are separated by 3 or more amino acid residues. In some embodiments, the dehydroalanine residue and the lysine residue are separated by 4 or more amino acid residues. In some embodiments, the dehydroalanine residue and the lysine residue are separated by 5 or more amino acid residues. In some embodiments, the dehydroalanine residue and the lysine residue are separated by 6 or more amino acid residues. In some embodiments, the dehydroalanine residue and the lysine residue are separated by 7 or more amino acid residues. In some embodiments, the dehydroalanine residue and the lysine residue are separated by 8 or more amino acid residues.

In some embodiments, the dehydroalanine residue and the lysine residue are separated by 1 to 8 amino acid residues. In some embodiments, the dehydroalanine residue and the lysine residue are separated by one amino acid residue. In some embodiments, the dehydroalanine residue and the lysine residue are separated by 2 amino acid residues. In some embodiments, the dehydroalanine residue and the lysine residue are separated by 3 amino acid residues. In some embodiments, the dehydroalanine residue and the lysine residue are separated by 4 amino acid residues. In some embodiments, the dehydroalanine residue and the lysine residue are separated by 5 amino acid residues. In some embodiments, the dehydroalanine residue and the lysine residue are separated by 6 amino acid residues. In some embodiments, the dehydroalanine residue and the lysine residue are separated by 7 amino acid residues. In some embodiments, the dehydroalanine residue and the lysine residue are separated by 8 amino acid residues.

In some embodiments, the dehydroalanine residue and the lysine residue are at (i, i+1) positions. In some embodiments, the dehydroalanine residue and the lysine residue are at (i, i+2) positions. In some embodiments, the dehydroalanine residue and the lysine residue are at (i, i+3) positions. In some embodiments, the dehydroalanine residue and the lysine residue are at (i, i+4) positions. In some embodiments, the dehydroalanine residue and the lysine residue are at (i, i+5) positions. In some embodiments, the dehydroalanine residue and the lysine residue are at (i, i+6) positions. In some embodiments, the dehydroalanine residue and the lysine residue are at (i, i+7) positions. In some embodiments, the dehydroalanine residue and the lysine residue are at (i, i+8) positions.

In some embodiments, the method further comprises contacting a peptide comprising a cysteine residue and a lysine residue with a reagent to convert the cysteine residue into a dehydroalanine residue. In some embodiments, the method further comprises contacting a peptide comprising a serine residue and a lysine residue with a reagent to convert the serine residue into a dehydroalanine residue. Any reagent useful for converting a cysteine residue or a serine residue into a dehydroalanine residue is contemplated by the present invention. In some embodiments, the reagent is selected from from O-mesitylenesulfonylhydroxylamine, 5,5-dithio-bis-(2-nitrobenzoic acid (Ellman's reagent), and 1,2-bis(bromomethyl)benzene. In some embodiments, the reagent comprises a 1,4-dihalobutane group. Non-limiting examples of reagents comprising a 1,4-dihalobutane group include 2,5-dibromohexanediamide, 1,4-butanediol dimethanesulfonate, 1,4-dibromobutane, 1,4-diiodobutane, and methyl 2,5-dibromopentanoate. See, for example, Chalker et al., Chemical Science 2011, 2:1666, which is incorporated by reference herein in its entirety.

In some embodiments, the method further comprises adding the reagent to a buffer solution. In some embodiments, the buffer solution is an aqueous buffer solution. In some embodiments, the buffer comprises one or more of guanidine hydrochloride and sodium hydrogen phosphate (Na₂HPO₄). In some embodiments, the buffer comprises guanidine hydrochloride and sodium hydrogen phosphate (Na₂HPO₄).

In some embodiments, the buffer solution is at a pH in a range of about 1 to about 12. In some embodiments, the buffer solution is at a pH in a range of about 4 to about 11. In some embodiments, the buffer solution is at a pH in a range of about 6 to about 10. In some embodiments, the buffer solution is at a pH in a range of about 8 to about 9. In some embodiments, the buffer solution is at a pH of about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, or about 12. In some embodiments, the buffer solution is at a pH of about 8. In some embodiments, the buffer solution is at a pH of about 8.5. In some embodiments, the buffer solution is at a pH of about 9.

In some embodiments, the method further comprises adding the peptide comprising a cysteine residue and a lysine residue to a buffer solution. In some embodiments, the buffer solution is an aqueous buffer solution. In some embodiments, the buffer comprises one or more of guanidine hydrochloride and sodium hydrogen phosphate (Na₂HPO₄). In some embodiments, the buffer comprises guanidine hydrochloride and sodium hydrogen phosphate (Na₂HPO₄).

In some embodiments, the buffer solution is at a pH in a range of about 1 to about 12. In some embodiments, the buffer solution is at a pH in a range of about 2 to about 6. In some embodiments, the buffer solution is at a pH in a range of about 2 to about 3. In some embodiments, the buffer solution is at a pH in a range of about 8 to about 9. In some embodiments, the buffer solution is at a pH of about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, or about 12. In some embodiments, the buffer solution is at a pH of about 2. In some embodiments, the buffer solution is at a pH of about 2.5. In some embodiments, the buffer solution is at a pH of about 3.

In some embodiments, the buffer solution has a concentration of guanidine hydrochloride of about 2 M to about 10 M. In some embodiments, the buffer solution has a concentration of guanidine hydrochloride of about 4 M to about 8 M. In some embodiments, the buffer solution has a concentration of guanidine hydrochloride of about 5 M to about 7 M. In some embodiments, the buffer solution has a concentration of guanidine hydrochloride of about 2 M, about 2.5 M, about 3 M, about 3.5 M, about 4 M, about 4.5 M, about 5 M, about 5.5 M, about 6 M, about 6.5 M, about 7 M, about 7.5 M, about 8 M, about 8.5 M, about 9 M, about 9.5 M, or about 10. In some embodiments, the the buffer solution has a concentration of guanidine hydrochloride of about 6 M.

In some embodiments, the buffer solution has a concentration of sulfuric acid of about 50 mM to about 150 mM. In some embodiments, the buffer solution has a concentration of sulfuric acid of about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, or about 150 mM. In some embodiments, the buffer solution has a concentration of sulfuric acid of about 100 mM.

In some embodiments, the method further comprises adding the buffer solution comprising the peptide comprising a cysteine residue and a lysine residue to the buffer solution comprising the reagent. In some embodiments, the method further comprises adding the buffer solution comprising the reagent to the buffer solution comprising the peptide comprising a cysteine residue and a lysine residue.

In some embodiments, the cysteine residue and the lysine residue are separated by one or more amino acid residues. In some embodiments, the cysteine residue and the lysine residue are separated by 2 or more amino acid residues. In some embodiments, the cysteine residue and the lysine residue are separated by 3 or more amino acid residues. In some embodiments, the cysteine residue and the lysine residue are separated by 4 or more amino acid residues. In some embodiments, the cysteine residue and the lysine residue are separated by 5 or more amino acid residues. In some embodiments, the cysteine residue and the lysine residue are separated by 6 or more amino acid residues. In some embodiments, the cysteine residue and the lysine residue are separated by 7 or more amino acid residues. In some embodiments, the cysteine residue and the lysine residue are separated by 8 or more amino acid residues.

In some embodiments, the cysteine residue and the lysine residue are separated by 1 to 8 amino acid residues. In some embodiments, the cysteine residue and the lysine residue are separated by one amino acid residue. In some embodiments, the cysteine residue and the lysine residue are separated by 2 amino acid residues. In some embodiments, the cysteine residue and the lysine residue are separated by 3 amino acid residues. In some embodiments, the cysteine residue and the lysine residue are separated by 4 amino acid residues. In some embodiments, the cysteine residue and the lysine residue are separated by 5 amino acid residues. In some embodiments, the cysteine residue and the lysine residue are separated by 6 amino acid residues. In some embodiments, the cysteine residue and the lysine residue are separated by 7 amino acid residues. In some embodiments, the cysteine residue and the lysine residue are separated by 8 amino acid residues.

In some embodiments, the cysteine residue and the lysine residue are at (i, i+1) positions. In some embodiments, the cysteine residue and the lysine residue are at (i, i+2) positions. In some embodiments, the cysteine residue and the lysine residue are at (i, i+3) positions. In some embodiments, the cysteine residue and the lysine residue are at (i, i+4) positions. In some embodiments, the cysteine residue and the lysine residue are at (i, i+5) positions. In some embodiments, the cysteine residue and the lysine residue are at (i, i+6) positions. In some embodiments, the cysteine residue and the lysine residue are at (i, i+7) positions. In some embodiments, the cysteine residue and the lysine residue are at (i, i+8) positions.

In some embodiments, the dithiocarbamate stapled peptide comprises a peptide backbone comprising 3 or more amino acid residues. In some embodiments, the dithiocarbamate stapled peptide comprises a peptide backbone comprising 4 or more amino acid residues. In some embodiments, the dithiocarbamate stapled peptide comprises a peptide backbone comprising 5 or more amino acid residues. In some embodiments, the dithiocarbamate stapled peptide comprises a peptide backbone comprising 6 or more amino acid residues. In some embodiments, the dithiocarbamate stapled peptide comprises a peptide backbone comprising 7 or more amino acid residues. In some embodiments, the dithiocarbamate stapled peptide comprises a peptide backbone comprising 8 or more amino acid residues. In some embodiments, the dithiocarbamate stapled peptide comprises a peptide backbone comprising 9 or more amino acid residues. In some embodiments, the dithiocarbamate stapled peptide comprises a peptide backbone comprising 10 or more amino acid residues. In some embodiments, the dithiocarbamate stapled peptide comprises a peptide backbone comprising 11 or more amino acid residues. In some embodiments, the dithiocarbamate stapled peptide comprises a peptide backbone comprising 12 or more amino acid residues. In some embodiments, the dithiocarbamate stapled peptide comprises a peptide backbone comprising 13 or more amino acid residues. In some embodiments, the dithiocarbamate stapled peptide comprises a peptide backbone comprising 14 or more amino acid residues. In some embodiments, the dithiocarbamate stapled peptide comprises a peptide backbone comprising 15 or more amino acid residues. In some embodiments, the dithiocarbamate stapled peptide comprises a peptide backbone comprising 16 or more amino acid residues. In some embodiments, the dithiocarbamate stapled peptide comprises a peptide backbone comprising 17 or more amino acid residues. In some embodiments, the dithiocarbamate stapled peptide comprises a peptide backbone comprising 18 or more amino acid residues. In some embodiments, the dithiocarbamate stapled peptide comprises a peptide backbone comprising 19 or more amino acid residues. In some embodiments, the dithiocarbamate stapled peptide comprises a peptide backbone comprising 20 or more amino acid residues.

In some embodiments, the peptide backbone comprises 3 to 20 amino acids. In some embodiments, the peptide backbone comprises 3 amino acid residues. In some embodiments, the peptide backbone comprises 4 amino acid residues. In some embodiments, the peptide backbone comprises 5 amino acid residues. In some embodiments, the peptide backbone comprises 6 amino acid residues. In some embodiments, the peptide backbone comprises 7 amino acid residues. In some embodiments, the peptide backbone comprises 8 amino acid residues. In some embodiments, the peptide backbone comprises 9 amino acid residues. In some embodiments, the peptide backbone comprises 10 amino acid residues. In some embodiments, the peptide backbone comprises 11 amino acid residues. In some embodiments, the peptide backbone comprises 12 amino acid residues. In some embodiments, the peptide backbone comprises 13 amino acid residues. In some embodiments, the peptide backbone comprises 14 amino acid residues. In some embodiments, the peptide backbone comprises 15 amino acid residues. In some embodiments, the peptide backbone comprises 16 amino acid residues. In some embodiments, the peptide backbone comprises 17 amino acid residues. In some embodiments, the peptide backbone comprises 18 amino acid residues. In some embodiments, the peptide backbone comprises 19 amino acid residues. In some embodiments, the peptide backbone comprises 20 amino acid residues.

### Stapled Peptides

In one aspect, the present disclosure provides a stapled peptide comprising a peptide backbone and a staple. In some embodiments, the peptide backbone comprises three or more amino acid residues. In some embodiments, the staple comprises a dithiocarbamate moiety and is attached to a cysteine residue and a lysine residue. In some embodiments, the cysteine and lysine residues are separated by one or more amino acid residues.

The term “staple” as used herein refers to the intramolecular or intermolecular connection (also referred to as cross-linking) of two peptides or two peptide domains (e.g., two loops of a helical peptide). When the peptide has a helical secondary structure, the staple is a macrocyclic ring, which is exogenous (not part of) core or inherent (non-stapled) helical peptide structure. In some embodiments, the macrocyclic ring is comprises one ore more dithiocarbamate moieties and incorporates at least two amino acids of the peptide. In some embodiments, the size of the macrocyclic ring is determined by the number of helical peptide amino acids in the ring and the number methylene groups in the moieties connecting the one ore more dithiocarbamate moieties to the peptide.

In some embodiments, the stapled peptides of the disclosure exhibit increased α-helical stability in aqueous solution compared to a corresponding non-stapled peptide. In some embodiments, the stapled peptide exhibits increased thermal stability compared to a corresponding non-stapled peptide. In some embodiments, the stapled peptide exhibits increased biological activity compared to a corresponding non-stapled polypeptide. In some embodiments, the stapled peptide exhibits increased resistance to proteolytic degradation compared to a corresponding non-stapled peptide. In some embodiments, the stapled peptide exhibits increased ability to penetrate living cells compared to a corresponding non-stapled peptide.

In some embodiments, the stapled peptide exhibits improved binding to p53 as compared to a non-stapled peptide and/or linear peptide. In some embodiments, the stapled peptide exhibits improved binding to MDM2 as compared to a non-stapled peptide and/or linear peptide. In some embodiments, the stapled peptide exhibits improved binding to MDMX as compared to a non-stapled peptide and/or linear peptide.

In some embodiments, the stapled peptide comprises two or more staples, wherein the staples are chemically similar or chemically different.

It will be appreciated that the number of crosslinking moieties (i.e. linkers or staples) is not limited to one or two, rather the number of crosslinking moieties utilized can be varied with the length of the targeting and/or effector domain as desired, and as compatible with the desired structure and activity to be generated.

In certain forms, the linkage is N-terminus to N-terminus. In certain forms, the linkage is C-terminus to N-terminus. In certain forms, the linkage is C-terminus to C-terminus. In still other forms, the linkage may be through interior amino acids of one or both peptides. As will be appreciated by one of ordinary skill in the art, the linkage is typically positioned in such a way as to avoid interfering with the binding activity of the peptide. The linkage may also be positioned in such a way to avoid interfering with the stapling of the peptide.

Alternatives to hydrophobic hydrocarbon linkers are provided herein. For example, the staple or linker can include one or more of an ether, thioether, ester, amine, or amide moiety. In some embodiments, a naturally occurring amino acid side chain can be incorporated into the linker. For example, a staple or linker can be coupled with a functional group which contains a chiral hydroxyl moiety present on serine, the thiol in cysteine, the primary amine in lysine, the acid in aspartate or glutamate, or the amide in asparagine or glutamine. In some embodiments, the staple is made by coupling two naturally occurring amino acids. In some embodiments, the staple is made by coupling two non-naturally occurring amino acids. In some embodiments, the staple is made by coupling a single non-naturally occurring amino acid together with a naturally occurring amino acid.

In some embodiments, the peptide backbone comprises 3 or more amino acid residues. In some embodiments, the peptide backbone comprises 4 or more amino acid residues. In some embodiments, the peptide backbone comprises 5 or more amino acid residues. In some embodiments, the peptide backbone comprises 6 or more amino acid residues. In some embodiments, the peptide backbone comprises 7 or more amino acid residues. In some embodiments, the peptide backbone comprises 8 or more amino acid residues. In some embodiments, the peptide backbone comprises 9 or more amino acid residues. In some embodiments, the peptide backbone comprises 10 or more amino acid residues. In some embodiments, the peptide backbone comprises 11 or more amino acid residues. In some embodiments, the peptide backbone comprises 12 or more amino acid residues. In some embodiments, the peptide backbone comprises 13 or more amino acid residues. In some embodiments, the peptide backbone comprises 14 or more amino acid residues. In some embodiments, the peptide backbone comprises 15 or more amino acid residues. In some embodiments, the peptide backbone comprises 16 or more amino acid residues. In some embodiments, the peptide backbone comprises 17 or more amino acid residues. In some embodiments, the peptide backbone comprises 18 or more amino acid residues. In some embodiments, the peptide backbone comprises 19 or more amino acid residues. In some embodiments, the peptide backbone comprises 20 or more amino acid residues.

In some embodiments, the peptide backbone comprises 3 to 20 amino acids. In some embodiments, the peptide backbone comprises 3 amino acid residues. In some embodiments, the peptide backbone comprises 4 amino acid residues. In some embodiments, the peptide backbone comprises 5 amino acid residues. In some embodiments, the peptide backbone comprises 6 amino acid residues. In some embodiments, the peptide backbone comprises 7 amino acid residues. In some embodiments, the peptide backbone comprises 8 amino acid residues. In some embodiments, the peptide backbone comprises 9 amino acid residues. In some embodiments, the peptide backbone comprises 10 amino acid residues. In some embodiments, the peptide backbone comprises 11 amino acid residues. In some embodiments, the peptide backbone comprises 12 amino acid residues. In some embodiments, the peptide backbone comprises 13 amino acid residues. In some embodiments, the peptide backbone comprises 14 amino acid residues. In some embodiments, the peptide backbone comprises 15 amino acid residues. In some embodiments, the peptide backbone comprises 16 amino acid residues. In some embodiments, the peptide backbone comprises 17 amino acid residues. In some embodiments, the peptide backbone comprises 18 amino acid residues. In some embodiments, the peptide backbone comprises 19 amino acid residues. In some embodiments, the peptide backbone comprises 20 amino acid residues.

In some embodiments, the cysteine residue and the lysine residue are separated by one or more amino acid residues. In some embodiments, the cysteine residue and the lysine residue are separated by 2 or more amino acid residues. In some embodiments, the cysteine residue and the lysine residue are separated by 3 or more amino acid residues. In some embodiments, the cysteine residue and the lysine residue are separated by 4 or more amino acid residues. In some embodiments, the cysteine residue and the lysine residue are separated by 5 or more amino acid residues. In some embodiments, the cysteine residue and the lysine residue are separated by 6 or more amino acid residues. In some embodiments, the cysteine residue and the lysine residue are separated by 7 or more amino acid residues. In some embodiments, the cysteine residue and the lysine residue are separated by 8 or more amino acid residues.

In some embodiments, the cysteine residue and the lysine residue are separated by 1 to 8 amino acid residues. In some embodiments, the cysteine residue and the lysine residue are separated by one amino acid residue. In some embodiments, the cysteine residue and the lysine residue are separated by 2 amino acid residues. In some embodiments, the cysteine residue and the lysine residue are separated by 3 amino acid residues. In some embodiments, the cysteine residue and the lysine residue are separated by 4 amino acid residues. In some embodiments, the cysteine residue and the lysine residue are separated by 5 amino acid residues. In some embodiments, the cysteine residue and the lysine residue are separated by 6 amino acid residues. In some embodiments, the cysteine residue and the lysine residue are separated by 7 amino acid residues. In some embodiments, the cysteine residue and the lysine residue are separated by 8 amino acid residues.

In some embodiments, the cysteine residue and the lysine residue are at (i, i+1) positions. In some embodiments, the cysteine residue and the lysine residue are at (i, i+2) positions. In some embodiments, the cysteine residue and the lysine residue are at (i, i+3) positions. In some embodiments, the cysteine residue and the lysine residue are at (i, i+4) positions. In some embodiments, the cysteine residue and the lysine residue are at (i, i+5) positions. In some embodiments, the cysteine residue and the lysine residue are at (i, i+6) positions. In some embodiments, the cysteine residue and the lysine residue are at (i, i+7) positions. In some embodiments, the cysteine residue and the lysine residue are at (i, i+8) positions.

In some embodiments, the stapled peptide is capped at the 5′ end, at the 3′ end, or at both ends. In some embodiments, the peptide is protected from proteolysis by “capping” the amino and/or carboxyl termini of the peptide. The term “capping” refers to the introduction of a blocking group at the end of a peptide via covalent modification. In some embodiments, the blocking group caps the end of the peptide without reducing the biological activity of the peptide. A non-limiting examples of a capping is amino-terminal acetylation of the disclosed peptide. Other capping portions are possible. In some embodiments, the choice of acylating moiety provides an opportunity to “cap” the peptide and to modulate the hydrophobicity of the compound. For example, the following series of acyl groups increases hydrophobicity and is also considered as a capping moiety: formyl, acetyl, propanoyl, hexanoyl, myristoyl. In some embodiments, the capping moiety comprises a fluorescent tag. Non-limiting examples of capping moieties comprising a fluorescent tag include fluorescein 5-isothiocyanate, fluorescein 6-isothiocyanate, and 5-carboxytetramethylrhodamine (TAMRA). In some embodiments, the stapled peptide is capped with an acetyl group. In some embodiments, the capping is carboxyl-terminal amidation. In some embodiments, the stapled peptide is capped at the 5′ end. In some embodiments, the stapled peptide is capped at the 3′ end. In some embodiments, the stapled peptide is capped at the 5′ end and at the 3′ end.

In some embodiments, the staple comprises a structure of formula (I):

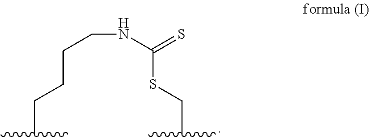 [SMILES: *CCCCNC(=S)SC*]

In some embodiments, the stapled peptide comprises a structure of formula (II) or formula (III):

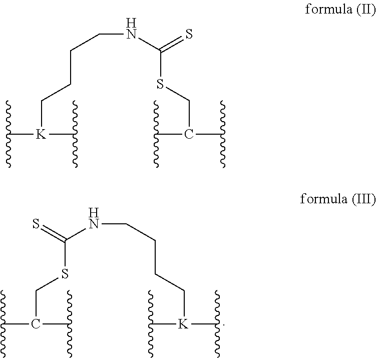

In some embodiments, the stapled peptide comprises a structure formula (11) or formula (12), wherein each of A¹, A², and A³ is independently selected from the residue of any amino acid described herein:

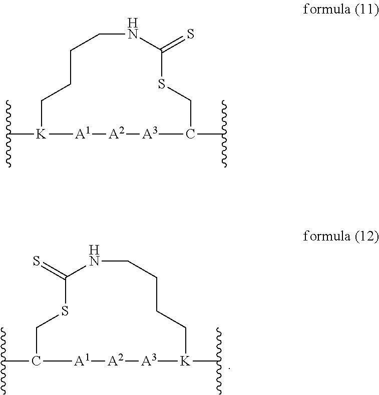

In some embodiments, the stapled peptide comprises a structure of any one of formulas (101) to formula (112):

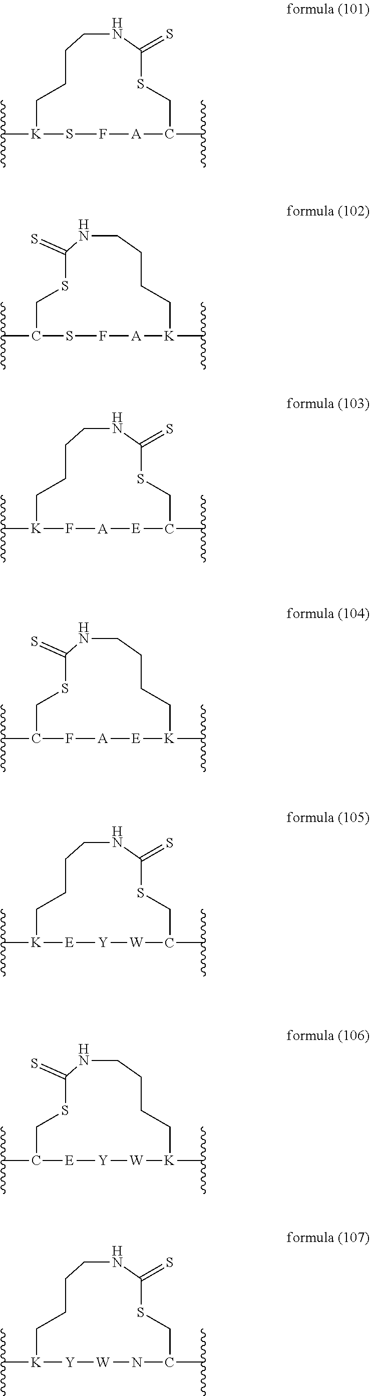

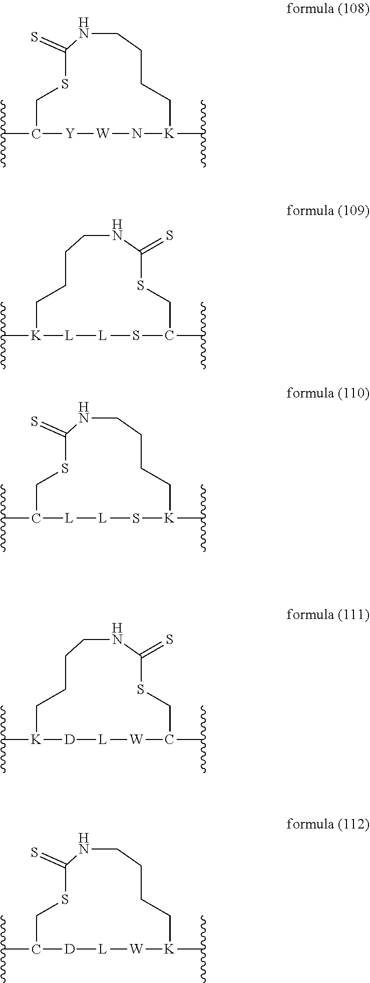

In some embodiments, the stapled peptide is selected from SEQ ID NO: 1 to SEQ ID NO: 11:

| Peptide No. | Peptide Structure |
|---|---|
| SEQ ID NO: 1 | 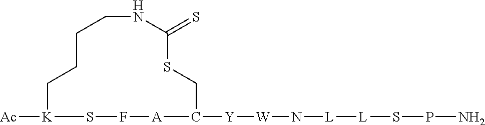 |
| SEQ ID NO: 2 | 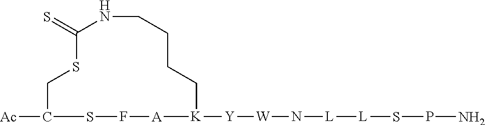 |
| SEQ ID NO: 3 | 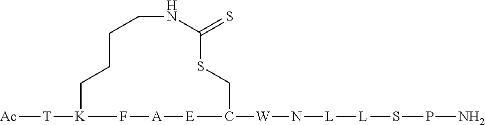 |
| SEQ ID NO: 4 | 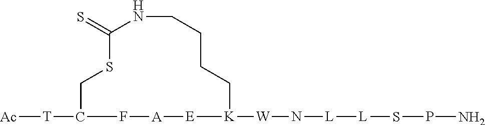 |
| SEQ ID NO: 5 | 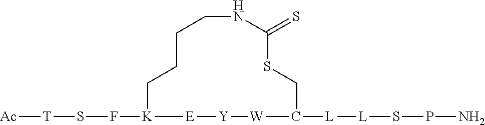 |
| SEQ ID NO: 6 | 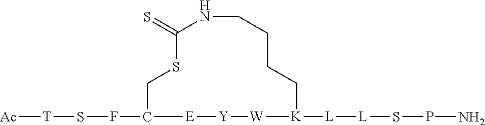 |
| SEQ ID NO: 7 | 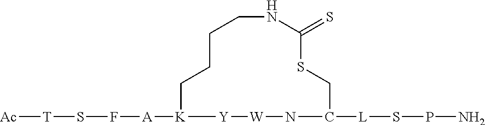 |
| SEQ ID NO: 8 | 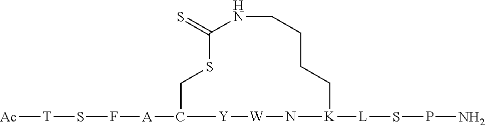 |
| SEQ ID NO: 9 | 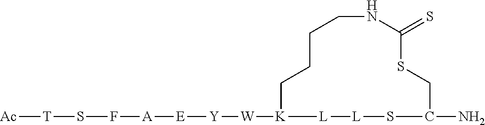 |
| SEQ ID NO: 10 | 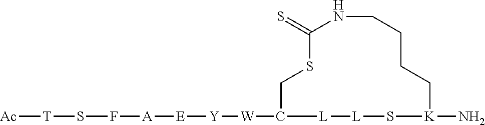 |
| SEQ ID NO: 11 | 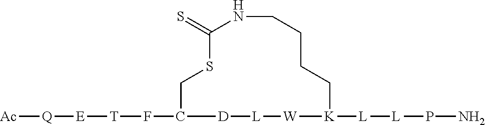 |

In some embodiments, the peptide is selected from SEQ ID NO: 5 and SEQ ID NO: 9:

| Peptide No. | Peptide Structure |
|---|---|
| SEQ ID NO: 5 | 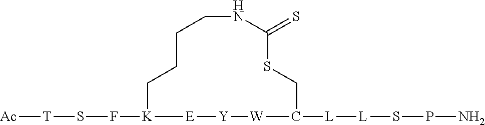 |
| SEQ ID NO: 9 | 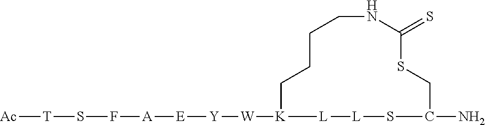 |

### Methods of Treatment

The compounds and compositions described herein can be used in methods for treating or preventing conditions and diseases, including but not limited to: a method of treating a condition associated with p53 activity, the method comprising administering to the patient a therapeutically effective amount of a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salts, solvates, hydrates, cocrystals, or prodrugs thereof; a method of treating a condition by inducing p53 activity in a patient in need of said treatment, the method comprising administering to the patient a therapeutically effective amount of a compound of a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salts, solvates, hydrates, cocrystals, or prodrugs thereof; a method of treating cancer in a patient in need of said treatment, the method comprising administering to the patient a therapeutically effective amount of a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salts, solvates, hydrates, cocrystals, or prodrugs thereof.

In some embodiments, the cancer is selected from bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thymoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophageal tumors, hematological neoplasms, non-small-cell lung cancer, chronic myelocytic leukemia, diffuse large B-cell lymphoma, esophagus tumor, follicle center lymphoma, head and neck tumor, hepatitis C virus induced cancer, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

In some embodiments, the methods of treatment include providing certain dosage amounts of a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salts, solvates, hydrates, cocrystals, or prodrugs thereof to a patient. In some embodiments, the dosage levels of each active agent of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single unit dosage form will vary depending upon the patient treated and the particular mode of administration.

### Pharmaceutical Compositions

In an embodiment, the disclosure provides a pharmaceutical composition for use in the treatment of the diseases and conditions described herein.

The pharmaceutical compositions are typically formulated to provide a therapeutically effective amount of a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salts, solvates, hydrates, cocrystals, or prodrugs thereof, as described herein, as the active ingredient. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

The pharmaceutical compositions described above are for use in the treatment or prevention of, without limitation, a condition associated with p53 activity, a condition associated with the inducement of p53 activity, and cancer, the pharmaceutical composition comprising one or more peptides of the disclosure such as, without limitation, any one of SEQ ID NOs: 1-11, or pharmaceutically acceptable salts, solvates, hydrates, cocrystals, or prodrugs thereof, provided in the pharmaceutical compositions of the disclosure is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, provided in the pharmaceutical compositions of the disclosure is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, provided in the pharmaceutical compositions of the disclosure is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, provided in the pharmaceutical compositions of the disclosure is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, provided in the pharmaceutical compositions of the disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, provided in the pharmaceutical compositions of the disclosure is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5 g, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the stapled peptides provided according to the disclosure is effective over a wide dosage range. For example, in the treatment of adult humans, dosages independently ranging from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

Described below are non-limiting pharmaceutical compositions and methods for preparing the same.

### Pharmaceutical Compositions for Oral Administration

In preferred embodiments, the disclosure provides a pharmaceutical composition for oral administration containing: one or more dithiocarbamate stapled peptides as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, and a pharmaceutical excipient suitable for administration.

The pharmaceutical compositions described above are preferably for use in the treatment of the diseases and conditions described below. In a preferred embodiment, the pharmaceutical compositions are for use in the treatment of cancer. In one embodiment, the pharmaceutical compositions of the present invention are for use in the treatment of a cancer selected from the group consisting of bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thymoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophageal tumors, hematological neoplasms, non-small-cell lung cancer, chronic myelocytic leukemia, diffuse large B-cell lymphoma, esophagus tumor, follicle center lymphoma, head and neck tumor, hepatitis C virus related cancer, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

In some embodiments, the pharmaceutical composition may be a liquid pharmaceutical composition suitable for oral consumption.

Pharmaceutical compositions of the disclosure suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, 6-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In preferred embodiments, the disclosure provides a pharmaceutical composition for injection containing: one or more stapled peptides including a structure of any one of formula (I), formula (II), formula (III), formula (11) or (12), or formula (101) to (112), such as, without limitation, a stapled peptide of any of SEQ ID NOs: 1-11, or pharmaceutically acceptable salt thereof, described herein, and a pharmaceutical excipient suitable for injection. Components and amounts of compounds in the compositions are as described herein.

The forms in which the compositions of the disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

Sterile injectable solutions are prepared by incorporating a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In preferred embodiments, the disclosure provides a pharmaceutical composition for transdermal delivery containing: a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, and a pharmaceutical excipient suitable for transdermal delivery.

Compositions of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present disclosure employs transdermal delivery devices (“patches”). Such transdermal patches may be used to provide continuous or discontinuous infusion of: a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445 and 5,001,139. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, et al., eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of a stapled peptide of the disclosure, e.g., a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, or a pharmaceutical composition of these compounds can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. A dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, can also be administered intraadiposally or intrathecally.

The compositions of the disclosure may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the disclosure may slow or inhibit the migration and proliferation of smooth muscle cells in the arterial wall which contribute to restenosis. A compound of the disclosure may be administered, for example, by local delivery from the struts of a stent, from a stent graft, from grafts, or from the cover or sheath of a stent. In some embodiments, a compound of the disclosure is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. A dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the disclosure in a suitable solvent, followed by evaporation of the solvent. Excess drug on the surface of the stent may be removed via an additional brief solvent wash. In yet other embodiments, compounds of the disclosure may be covalently linked to a stent or graft. A covalent linker may be used which degrades in vivo, leading to the release of the compound of the disclosure. Any bio-labile linkage may be used for such a purpose, such as ester, amide or anhydride linkages. A dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, may additionally be administered intravascularly from a balloon used during angioplasty. Extravascular administration of a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, via the pericard or via advential application of formulations of the disclosure may also be performed to decrease restenosis.

Exemplary parenteral administration forms include solutions or suspensions of a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, in sterile aqueous solutions, for example, aqueous propylene glycol or dextrose solutions. Such dosage forms can be suitably buffered, if desired.

The disclosure also provides kits. The kits include a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In some embodiments, the stapled peptide including a structure of any one of formula (I), formula (II), formula (III), formula (11) or (12), or formula (101) to (112), such as, without limitation, a stapled peptide of any of SEQ ID NOs: 1-11, or pharmaceutically acceptable salt thereof, described herein, and another active pharmaceutical ingredient are provided as separate compositions in separate containers within the kit. In some embodiments, the stapled peptide including a structure of any one of formula (I), formula (II), formula (III), formula (11) or (12), or formula (101) to (112), such as, without limitation, a stapled peptide of any of SEQ ID NOs: 1-11, or pharmaceutically acceptable salt thereof, and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

The kits described above are preferably for use in the treatment of the diseases and conditions described herein. In a preferred embodiment, the kits are for use in the treatment of cancer. In some embodiments, the kits are for use in treating solid tumor cancers, lymphomas and leukemias.

In some embodiments, the kits of the present invention are for use in the treatment of a cancer selected from the group consisting of bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thymoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophageal tumors, hematological neoplasms, non-small-cell lung cancer, chronic myelocytic leukemia, diffuse large B-cell lymphoma, esophagus tumor, follicle center lymphoma, head and neck tumor, hepatitis C virus related cancer, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

### Dosages and Dosing Regimens

The amounts of: a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, administered will be dependent on the human or mammal being treated, the severity of the disorder or condition, the rate of administration, the disposition of the compounds and the discretion of the prescribing physician. However, an effective dosage of each is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, may be provided in units of mg/kg of body mass or in mg/m² of body surface area.

In some embodiments, a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein is administered in multiple doses. In a preferred embodiment, a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein is administered in multiple doses. Dosing may be once, twice, three times, four times, five times, six times, or more than six times per day. Dosing may be once a month, once every two weeks, once a week, or once every other day. In other embodiments, a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, is administered about once per day to about 6 times per day. In some embodiments, a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, is administered once daily, while in other embodiments, a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein is administered twice daily, and in other embodiments a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, is administered three times daily.

Administration a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, may continue as long as necessary. In some embodiments, a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, is administered for more than 1, 2, 3, 4, 5, 6, 7, 14, or 28 days. In some embodiments, a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein is administered for less than 28, 14, 7, 6, 5, 4, 3, 2, or 1 day. In some embodiments, a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein is administered chronically on an ongoing basis—e.g., for the treatment of chronic effects. In another embodiment, the administration of a stapled peptide of the disclosure such as SEQ ID NOs: 1-11, or pharmaceutically acceptable salt thereof, described herein, continues for less than about 7 days. In yet another embodiment, the administration continues for more than about 6, 10, 14, 28 days, two months, six months, or one year. In some cases, continuous dosing is achieved and maintained as long as necessary.

In some embodiments, an effective dosage of a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg.

In some embodiments, an effective dosage of a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some instances, dosage levels below the lower limit of the aforesaid ranges may be more than adequate, while in other cases still larger doses may be employed without causing any harmful side effect—e.g., by dividing such larger doses into several small doses for administration throughout the day.

An effective amount of a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

Methods of Treating Solid Tumor Cancers, Hematological Malignancies, Inflammation, Immune and Autoimmune Disorders, and Other Diseases

An effective amount of a dithiocarbamate stapled peptide as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or pharmaceutically acceptable salt thereof, described herein, may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

In some embodiments, the invention relates to a method of treating a condition by inducing p53 activity in a patient in need thereof, including administering to the patient a therapeutically effective amount of one or more dithiocarbamate stapled peptides as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or a pharmaceutically acceptable salt, solvate, hydrate, cocrystal, or prodrug thereof.

In a preferred embodiment, the patient or subject is a mammal, such as a human. In an embodiment, the patient or subject is a human. In an embodiment, the patient or subject is a companion animal. In an embodiment, the patient or subject is a canine, feline, or equine.

In some embodiments, the invention relates to a method of treating a condition by inducing p53 activity, in a patient in need thereof, including administering to the patient dosage unit form including a therapeutically effective amount of one or more dithiocarbamate stapled peptides as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or a pharmaceutically acceptable salt, solvate, hydrate, cocrystal, or prodrug thereof. In some embodiments, the dosage unit form includes a physiologically compatible carrier medium.

In some embodiments, the invention relates to a method of treating a cancer by inducing p53 activity, in a patient in need thereof, including administering to the patient a therapeutically effective amount of one or more dithiocarbamate stapled peptides as described herein, including, without limitation, a stapled peptide comprising a structure of any one of formula (I), formula (II), formula (III), formula (11) or (12), or formula (101) to (112), and/or a stapled peptide having any one of SEQ ID NOs: 1-11, or a pharmaceutically acceptable salt, solvate, hydrate, cocrystal, or prodrug thereof.

In some embodiments, the cancer can be bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thymoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophageal tumors, hematological neoplasms, non-small-cell lung cancer, chronic myelocytic leukemia, diffuse large B-cell lymphoma, esophagus tumor, follicle center lymphoma, head and neck tumor, hepatitis C virus induced cancer, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

Efficacy of the methods, compounds, and combinations of compounds described herein in treating, preventing and/or managing the indicated diseases or disorders can be tested using various animal models known in the art. Efficacy in treating, preventing and/or managing asthma can be assessed using the ova induced asthma model described, for example, in Lee, et al., J. Allergy Clin. Immunol. 2006, 118, 403-9. Efficacy in treating, preventing and/or managing arthritis (e.g., rheumatoid or psoriatic arthritis) can be assessed using the autoimmune animal models described in, for example, Williams, et al., Chem. Biol. 2010, 17, 123-34, WO 2009/088986, WO 2009/088880, and WO 2011/008302. Efficacy in treating, preventing and/or managing psoriasis can be assessed using transgenic or knockout mouse model with targeted mutations in epidermis, vasculature or immune cells, mouse model resulting from spontaneous mutations, and immuno-deficient mouse model with xenotransplantation of human skin or immune cells, all of which are described, for example, in Boehncke, et al., Clinics in Dermatology, 2007, 25, 596-605. Efficacy in treating, preventing and/or managing fibrosis or fibrotic conditions can be assessed using the unilateral ureteral obstruction model of renal fibrosis, which is described, for example, in Chevalier, et al., Kidney International 2009, 75, 1145-1152; the bleomycin induced model of pulmonary fibrosis described in, for example, Moore, et al., Am. J. Physiol. Lung. Cell. Mol. Physiol. 2008, 294, L152-L160; a variety of liver/biliary fibrosis models described in, for example, Chuang, et al., Clin. Liver Dis. 2008, 12, 333-347 and Omenetti, et al., Laboratory Investigation, 2007, 87, 499-514 (biliary duct-ligated model); or any of a number of myelofibrosis mouse models such as described in Varicchio, et al., Expert Rev. Hematol. 2009, 2(3), 315-334. Efficacy in treating, preventing and/or managing scleroderma can be assessed using a mouse model induced by repeated local injections of bleomycin described, for example, in Yamamoto, et al., J. Invest. Dermatol. 1999, 112, 456-462. Efficacy in treating, preventing and/or managing dermatomyositis can be assessed using a myositis mouse model induced by immunization with rabbit myosin as described, for example, in Phyanagi, et al., Arthritis & Rheumatism, 2009, 60(10), 3118-3127. Efficacy in treating, preventing and/or managing lupus can be assessed using various animal models described, for example, in Ghoreishi, et al., Lupus, 2009, 19, 1029-1035; Ohl, et al., J. Biomed. Biotechnol., 2011, Article ID 432595; Xia, et al., Rheumatology, 2011, 50, 2187-2196; Pau, et al., PLoS ONE, 2012, 7(5), e36761; Mustafa, et al., Toxicology, 2011, 290, 156-168; Ichikawa, et al., Arthritis & Rheumatism, 2012, 62(2), 493-503; Rankin, et al., J. Immunology, 2012, 188, 1656-1667. Efficacy in treating, preventing and/or managing Sjögren's syndrome can be assessed using various mouse models described, for example, in Chiorini, et al., J. Autoimmunity, 2009, 33, 190-196. Models for determining efficacy of treatments for pancreatic cancer are described in Herreros-Villanueva, et al., World J. Gastroenterol. 2012, 18, 1286-1294. Models for determining efficacy of treatments for breast cancer are described, e.g., in Fantozzi, Breast Cancer Res. 2006, 8, 212. Models for determining efficacy of treatments for ovarian cancer are described, e.g., in Mullany, et al., Endocrinology 2012, 153, 1585-92; and Fong, et al., J. Ovarian Res. 2009, 2, 12. Models for determining efficacy of treatments for melanoma are described, e.g., in Damsky, et al., Pigment Cell & Melanoma Res. 2010, 23, 853-859. Models for determining efficacy of treatments for lung cancer are described, e.g., in Meuwissen, et al., Genes & Development, 2005, 19, 643-664. Models for determining efficacy of treatments for lung cancer are described, e.g., in Kim, Clin. Exp. Otorhinolaryngol. 2009, 2, 55-60; and Sano, Head Neck Oncol. 2009, 1, 32. Models for determining efficacy of treatments for colorectal cancer, including the CT26 model, are described in Castle, et al., BMC Genomics, 2013, 15, 190; Endo, et al., Cancer Gene Therapy, 2002, 9, 142-148; Roth et al., Adv. Immunol. 1994, 57, 281-351; Fearon, et al., Cancer Res. 1988, 48, 2975-2980. Efficacy in DLBCL may be assessed using the PiBCL1 murine model and BALB/c (haplotype H-2^{d}) mice. Illidge, et al., Cancer Biother. & Radiopharm. 2000, 15, 571-80. Efficacy in NHL may be assessed using the 38C13 murine model with C3H/HeN (haplotype 2-H^{k}) mice or alternatively the 38C13 Her2/neu model. Timmerman, et al., Blood, 2001, 97, 1370-77; Penichet, et al., Cancer Immunolog. Immunother. 2000, 49, 649-662. Efficacy in CLL may be assessed using the BCL1 model using BALB/c (haplotype H-2^{d}) mice. Dutt, et al., Blood, 2011, 117, 3230-29.

Stapled Peptides with Chemotherapeutic Active Pharmaceutical Ingredients

Stapled peptides described herein can also be co-administered with additional chemotherapeutic active pharmaceutical ingredients, for example gemcitabine, albumin-bound paclitaxel (nab-paclitaxel), and bendamustine or bendamustine hydrochloride. In a preferred embodiment, the invention provides a method of treating a hematological malignancy or a solid tumor cancer in a human including the step of administering to said human a stapled peptide of the disclosure, and further including the step of administering a therapeutically-effective amount of gemcitabine, or a pharmaceutically acceptable salt, prodrug, cocrystal, solvate or hydrate thereof. In an embodiment, the invention provides a method of treating a hematological malignancy or a solid tumor cancer in a human including the step of administering to said human a stapled peptide of the disclosure described herein, or a pharmaceutically acceptable salt, prodrug, cocrystal, solvate or hydrate thereof, and further including the step of administering a therapeutically-effective amount of gemcitabine, or a pharmaceutically acceptable salt, prodrug, cocrystal, solvate or hydrate thereof. In an embodiment, the solid tumor cancer in any of the foregoing embodiments is pancreatic cancer.

In any of the foregoing embodiments, the chemotherapeutic active pharmaceutical ingredient or combinations thereof may be administered before, concurrently, or after administration of the human a stapled peptide described herein.

While preferred embodiments of the invention are shown and described herein, such embodiments are provided by way of example only and are not intended to otherwise limit the scope of the invention. Various alternatives to the described embodiments of the invention may be employed in practicing the invention.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Dithiocarbamate-Inspired Side Chain Stapling Chemistry for Peptide Drug Design

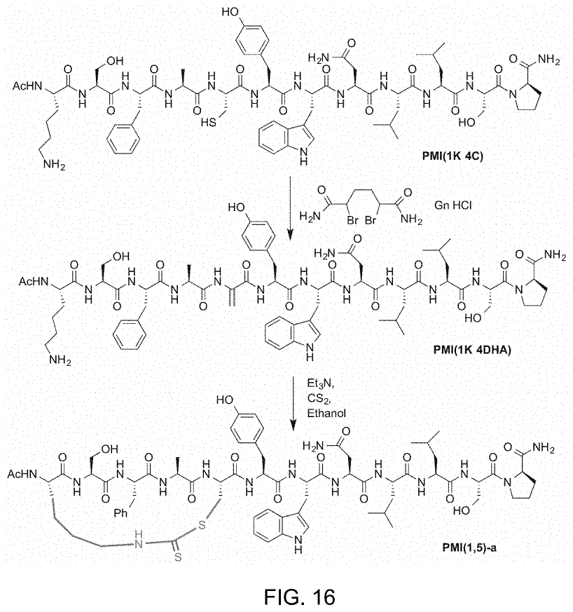

This Example describes a novel peptide stapling strategy based on the dithiocarbamate chemistry linking the side chains of residues Lys (i) and Cys (i+4) in a dodecameric peptide antagonist, termed PMI, of the p53-inhibitory oncogenic proteins MDM2 and MDMX. One dithiocarbamate-stapled PMI derivative, ^{DTC}PMI, showed a 50-fold stronger binding to MDM2 and MDMX than its linear counterpart. Crystallographic studies of peptide-MDM2/MDMX complexes structurally validated the design of the dithiocarbamate staple bridging Lys and Cys at (i, i+4) positions. Importantly, in contrast to PMI and its linear derivatives, the ^{DTC}PMI peptide actively traversed the cell membrane and killed HCT116 tumor cells in vitro by activating the tumor suppressor protein p53. See, for example, FIG. 17. This facile and cost-effective stapling chemistry is an important new tool for the development of peptide therapeutics with improved pharmacological properties.

Methods

Peptide and protein synthesis. All peptides and proteins used in this work were chemically synthesized, either in a stepwise fashion or via native chemical ligation. Peptides were synthesized using a machine-assisted Boc chemistry tailored from the optimized HBTU activation/DIEA in situ neutralization protocol. After chain assembly, side chain protecting groups were removed and peptides cleaved from the resin by treatment with anhydrous HF and p-cresol (9:1) at 0° C. for 1 h. Crude peptides were precipitated with cold ether and purified by preparative C18 reversed-phase (RP) HPLC. The synthesis of ²⁵⁻¹⁰⁹MDM2 and ²⁴⁻¹⁰⁸MDMX was described previously, and obtained via native chemical ligation. The reaction between MDM2(25-76)-COSR and MDM2(77-109) (1.5 eq) or between MDMX (24-75)-COSR and MDMX (76-108) (1.5 eq) was carried out at a total peptide concentration of 10-20 mg/ml in 0.25 M phosphate buffer (pH 7.1) containing 6 M guanidine hydrochloride, 50 mM MPAA and 20 mM TCEP.HCl. They went to completion overnight as monitored by analytical HPLC. The ligation products were purified by preparative RP-HPLC to homogeneity. The molecular masses of all peptides and proteins were ascertained by electrospray ionization mass spectrometry.

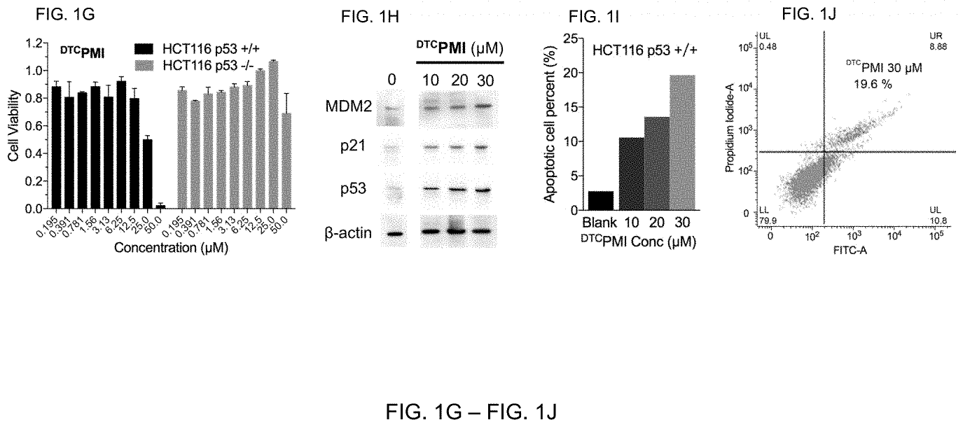

Synthesis of Stapled PMIs. PMI(1,5)-a is used as an example (FIG. 2). Cys to Dha. Buffer A containing 6 M guanidine hydrochloride and 100 mM Na₂HPO₄, pH=8.5, and Buffer B containing 6 M guanidine hydrochloride and 100 mM NaH₂PO₄, pH=2.5, were prepared prior to the reaction. 3 mL Buffer B was used for dissolving 50 mg PMI(1K,5C) for storage. 75 mg Bisamide reagent (1.5 mg per linear peptide) was dissolved in 47 mL Buffer A, followed by a slow addition of Buffer B containing the linear peptide. The reaction was stirred at room temperature overnight and monitored by analytical HPLC. The crude intermediate product PMI(1K,5DHA) was purified by preparative RP-HPLC to homogeneity (35 mg). DTC cyclization. 20 mg PMI(1K,5DHA) was dissolved in 10 mL ethanol, followed by addition of 1 mL Et₃N and 1 mL CS₂. The reaction proceeded with stirring overnight at room temperature until a complete conversion. After the solvent was removed, the residual material was purified by preparative RP-HPLC to yield the stapled product PMI(1,5)-a (10 mg).

Surface plasmon resonance (SPR). Competition binding kinetics was carried out at 25° C. using a Biacore T100 SPR instrument and ¹⁵⁻²⁹p53-immobilized CM5 sensor chips as described. ²⁵⁻¹⁰⁹MDM2 and ²⁴⁻¹⁰⁸MDMX at 50 nM or 100 nM were incubated in 10 mM HEPES buffer containing 150 mM NaCl, 0.005% surfactant P20, pH 7.4, with varying concentrations of peptide inhibitor before SPR analysis. The concentration of unbound MDM2 or MDMX in solution was deduced, based on p53-association RU values, from a calibration curve established by RU measurements of different concentrations of MDM2/MDMX injected alone. Two replicates and three independent experiments were performed.

Fluorescence polarization (FP). A FP-based competitive binding assay was established using ²⁵⁻¹⁰⁹MDM2, ²⁴⁻¹⁰⁸MDMX and a fluorescently tagged PMI peptide as previously described. Succinimidyl ester-activated carboxytetramethylrhodamine (TAMRA-NHS) was covalently conjugated to the N-terminus of PMI (TSFAEYWNLLSP) (K_{d}^{PMI-MDM2}=3.2 nM, K_{d}^{PMI-MDMX}=8.5 nM). Unlabeled PMI competed with TAMRA-PMI for MDM2/MDMX binding, based on which the K_{d} values of TAMRA-PMI with MDM2 and MDMX were determined by changes in FP to be 0.62 and 0.72 nM, respectively. As an additional positive control, the binding of Nutlin-3 to MDM2 and MDMX was quantified, yielding respective Ki values of 5.1 nM and 1.54 μM, similar to the values reported in the literature. For dose-dependent competitive binding experiments, MDM2 or MDMX protein (50 nM) was first incubated with TAMRA-PMI peptide (10 nM) in PBS (pH 7.4) on a Costar 96-well plate, to which a serially diluted solution of test peptide was added to a final volume of 125 μL. After 30 min of incubation at room temperature, the FP values were measured at λₑₓ=530 nm and λₑₘ=580 nm on a Tecan Infinite M1000 plate reader. Curve fitting was performed using GRAPHPAD PRISM software, and Ki values were calculated as described previously. Two replicates and three independent experiments were performed.

Cell Viability Assay. The human colon cancer cell lines HCT116 p53^{+/+} and HCT116 p53^{-/-}, and maintained in McCoy's 5 A medium (Invitrogen) supplemented with 10% heat-inactivated FCS and 1% penicillin-streptomycin at 37° C. with 5% CO₂ under fully humidified conditions. Cells (3×10³ cells/well) were seeded at in 96-well plates and treated with PMI and stapled PMIs at various concentrations in serum-free media for 8 hours, followed by serum complementary and additional incubation for 64 hours. The absorbance at 450 nm was then measured followed by the addition of CCK8 kit, and percent cell viability was calculated on the ratio of the A₄₅₀ of sample wells versus reference wells.

Circular Dichroism (CD) Spectroscopy. Compounds were dissolved in PB (pH=7.2) to concentrations ranging from 10-50 μM. The spectra were obtained on a Jasco J-715 spectropolarimeter at 20° C. The spectra were collected using a 0.1 cm path-length quartz cuvette with the following measurement parameters: wavelength, 185-255 nm; step resolution 0.1 nm; speed, 20 nm min⁻¹; accumulations, 6; bandwidth, 1 nm. The helical content of each peptide was calculated as reported previously.

Proteolytic Stability. PMI-0 and the stapled peptide PMI(8,12)-a were incubated at 100 μM each in RPMI 1640 with 25 μg/ml cathepsin G—an intracellular protease with dual specificities for both basic and bulky hydrophobic residues. RP-HPLC was used to monitor and quantify time-dependent peptide hydrolysis.

Stability in GSH. PMI(8,12)-a was incubated at 25° C. in PBS buffer with reduced glutathione at 10 mM. RP-HPLC and ESI-MS were used to monitor and quantify time-dependent breakdown of the DTC staple.

Western Blot Analysis. HCT116 p53^{+/+} cells (1×10⁶) incubated at 37° C. were treated with ^{DTC}P1V11 (10, 20, 30 μM) in serum-free media for 8 hours. The cells were lysed (20 mM Tris-HCl pH 8.0, 0.8% SDS, 1 mM PMSF, 1 U mL⁻¹ benzonase nuclease) and the crude lysates were clarified by brief centrifugation and total protein concentration was determined by using the Pierce BCA protein assay. Aliquots containing 5 μg of total protein were run on 4-12% Bis-Tris polyacrylamide gels (Invitrogen). Proteins were detected by chemiluminescence reagent (Perkin Elmer) using antibodies specific for p53 (Santa Cruz Biotechnology), MDM2 (Santa Cruz Biotechnology), p21 (Merck Millipore), and β-actin (Sigma-Aldrich).

Cell Apoptosis Assay. HCT116 p53^{+/+} cells were seeded in 6-well tissue culture plates (3×10⁵ cells per well) for 12 h and treated with 10, 20, or 30 μM ^{DTC}PMI in serum-free media for 8 hours, followed by serum complementary and additional incubation for 40 hours. No treatment controls were established. Culture medium that may contain detached cells was collected, and attached cells were trypsinized. After centrifugation and removal of the supernatants, cells were resuspended in 300 μL of 1×binding buffer which was then added to 5 μL of annexin V-FITC and incubated at room temperature for 15 min. After addition of 10 μL of PI, the cells were incubated at room temperature for another 10 min in the dark. The stained cells were analyzed by a flow cytometer (BD-FACSVerse).

Crystallization of stapled-PMI complexes. Initial screening for crystals was done with an Art Robbinson crystallization robot using vapor diffusion sitting trials of sparse matrix crystallization screens: the Hampton crystal screen I and II (Hampton Research), the precipitant wizard screen (Emerald BioSystems), the synergy screen (Emerald BioSystems) and the ProComplex and MacroSol screens from Molecular Dimensions. All crystallization experiments were performed with complexes at 8-10 mg/ml in 20 mM Tris pH 7.4. Conditions that produced micro crystals were then reproduced and optimized using the hanging-drop vapor diffusion method (drops of 0.5 μl of protein and 0.5 μl of precipitant solution equilibrated against 700 μl of reservoir solution). Diffraction quality crystals for MDM2-PMI(8,12)-a complex were obtained from a solution containing 1.34 M ammonium sulfate, 6.7% (v/v) glycerol, 50 mM magnesium sulfate, and 0.1 M imidazole pH 6.5. Prior to being frozen, the crystals were transferred into the crystal growth solution supplemented with 20% (v/v) 2-methyl-2,4-pentanediol (MPD). Crystals of MDMX-PMI(4,8)-a complex were grown from 30% (v/v) 2-propanol, 30% (v/v) PEG 3350, and 0.1 M Tris-HCl pH 8.5 and frozen from the same solution supplemented with 20% (v/v) MPD.

Data Collection, Structure Solution and Refinement. Diffraction data for both complexes were collected at the Stanford Synchrotron Radiation Light Source (SSRL) BL12-2 beam line equipped with Pilatus 6M PAD area detector. The MDM2-PMI(8,12)-a complex crystals belong to a space group C222₁ with unit-cell parameters a=90.8 Å, b=157.5 Å, and c=196.7 Å with twelve complexes copies present in the asymmetric unit. The MDMX-PMI(4,8)-a complex crystals belong to a space group P1 with unit-cell parameters a=43.3 Å, b=47.7 Å, c=93.4 Å, α=76.7°, β=89.9°, and γ=72.6° and eight complexes in the asymmetric unit (Table S3). The data for both the complexes were processed and scaled with HKL2000.⁶ Structures were solved by molecular replacement with Phaser from the CCP4 program suite based on the coordinates extracted from the structure of MDM2-PMI complex (PDB code: 3EQS) and MDMX-PMI complex (PDB code: 3EQY). The models were refined using Refmac and the structure manually rebuilt with COOT. The MDM2-PMI(8,12)-a complex was refined to R_{factor} of 0.197 and R_{free} of 0.245. The MDMX-PMI(4,8)-a complex was refined to R_{factor} of 0.278 and R_{free} of 0.336. 97.8% and 98.8% of residues fell within allowed regions of the Ramachandran plot as determined by MolProbity, respectively, as shown in Table 1 below.

TABLE 1
Data collection and refinement statistics

| Data collection | MDM2-PMI(8,12)-a | | MDMX-PMI(4,8)-a | |
|---|---|---|---|---|
| Wavelength, \acute{Å} | 0.97946 | | 0.97946 | |
| Space group | C222₁ | | P1 | |
| Cell parameters | | | | |
| a, b, c, Å | 90.8, 157.5, 196.7 | | 43.3, 47.7, 93.4 | |
| α, β, γ, ° | 90.0, 90.0, 90.0 | | 76.7, 89.9, 72.6 | |
| Complexes/a.u. | 12 | | 8 | |
| Resolution, (Å) | 50-1.8 | (1.83-1.8) | 50-2.7 | (2.75-2.7) |
| # of reflections | | | | |
| Total | 419,465 | | 31,027 | |
| Unique | 123,372 | | 16,330 | |
| R_{merg}^{b}, % | 7.6 | (29.9) | 13.2 | (61.4) |
| I/σ | 23.2 | (3.0) | 10.3 | (1.1) |
| Completeness, % | 95.5 | (97.9) | 86.0 | (78.7) |
| Redundancy | 3.4 | (3.4) | 1.9 | (1.8) |

Refinement Statistics

| | | |
|---|---|---|
| Resolution, Å | 41.7-1.80 | 50-2.7 |
| R^{c}, % | 19.8 | 28.1 |
| R_{free}^{d}, % | 24.6 | 33.5 |
| # of atoms | | |
| Protein | 9,547 | 5,910 |
| Water | 418 | 27 |
| Ligand/Ion | 182 | 115 |
| Overall B value (Å)² | | |
| Protein | 26.8 | 54.4 |
| Water | 24.8 | 38.9 |
| Ligand/Ion | 26.7 | 57.7 |
| Root mean square | | |
| deviation | | |
| Bond lengths, Å | 0.012 | 0.006 |
| Bond angles, ° | 1.79 | 1.14 |
| Ramachandran ^{e} | | |
| favored, % | 91.2 | 95.5 |
| allowed, % | 6.6 | 3.3 |
| outliers, % | 2.2 | 1.2 |
| PDB ID | 5VK0 | 5VK1 |

^{a}all data (outer shell).
^{b}R_{merge} = Σ|I − <I>|/ΣI, where I is the observed intensity and <I> is the average intensity obtained from multiple observations of symmetry-related reflections after rejections
^{c}R = Σ||Fₒ| − |F_{c}||/Σ|Fₒ|, where Fₒ and F_{c} are the observed and calculated structure factors, respectively
^{d}R_{free} as defined by Brünger

### Discussion

This highly efficient solution chemistry for unprotected peptides entails the conversion of Cys via oxidative elimination to dehydroalanine (DHA), which subsequently reacts with the C-amino group of Lys in the presence of carbon disulfide (CS₂). In this proof-of-concept study, PMI was used—a potent dodecameric peptide antagonist of MDM2 and MDMX that, despite its high affinity for both proteins, fails to activate p53 and kill p5.3^{+/+} tumor cells due presumably to its inability to traverse the cell membrane and susceptibility to proteolytic degradation.

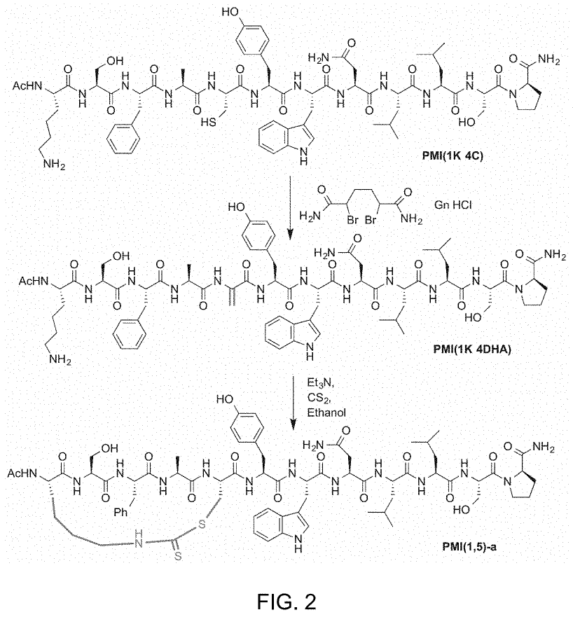

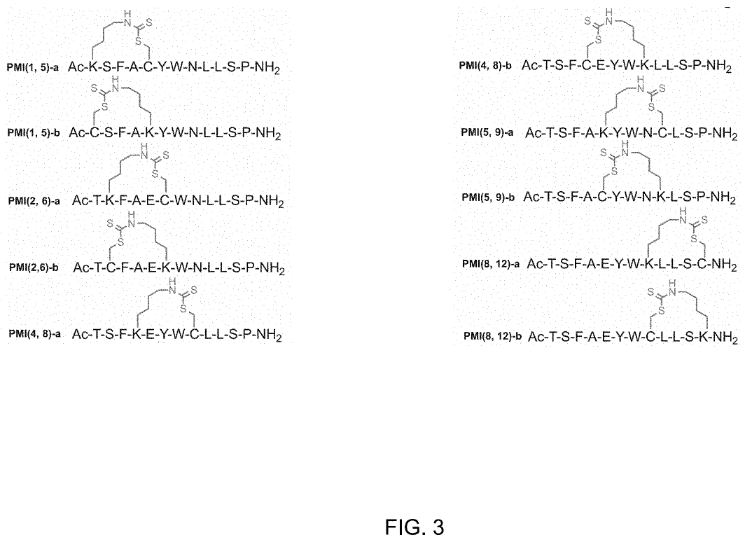

Previous structural and functional studies of PMI (TSFAEYWNLLSP) identified Phe3, Tyr6, Trp7 and Leu10 as the most critical residues for MDM2/MDMX binding. Thus, those four residues were maintained in the design of DTC-stapled peptides and introduced Lys-Cys (a) or Cys-Lys (b) pairs into (1, 5), (2, 6), (4, 8), (5, 9), or (8, 12) positions of PMI (FIG. 3). These N-acetylated and C-amidated peptides were synthesized using Boc-chemistry for solid phase peptide synthesis, and purified by HPLC to homogeneity. Conversion of Cys to DHA, monitored by HPLC and electrospray ionization mass spectrometry (ESI-MS), was achieved in an overnight reaction in 6 M GuHCl, pH 8.0, in the presence of the bisamide of the 1,4-dibromobutane core, followed by HPLC purification. Crosslinking DHA and Lys side chains was readily accomplished overnight in ethanol containing Et₃N and CS₂, as verified by ESI-MS (FIG. 4), resulting in 10 DTC-stapled constructs termed PMI(1,5)-a, PMI(1,5)-b, PMI(2,6)-a, PMI(2,6)-b, PMI(4,8)-a, PMI(4,8)-b, PMI(5,9)-a, PMI(5,9)-b, PMI(8,12)-a and PMI(8,12)-b (FIG. 3). Table 2 below shows the mass spectrometry data.

TABLE 2
Mass spectrometry data for DTC-stapled PMI peptides.

| Compound | Calculated Mass | Found Mass |
|---|---|---|
| PMI(1, 5)-a | 1511.78 | 1511.62 |
| PMI(1, 5)-b | 1511.78 | 1511.96 |
| PMI(2, 6)-a | 1491.75 | 1491.83 |
| PMI(2, 6)-b | 1491.75 | 1491.77 |
| PMI(4, 8)-a | 1556.82 | 1556.82 |
| PMI(4, 8)-b | 1556.82 | 1556.89 |
| PMI(5, 9)-a | 1499.73 | 1499.74 |
| PMI(5, 9)-b | 1499.73 | 1499.61 |
| PMI(8, 12)-a | 1530.78 | 1530.76 |
| PMI(8, 12)-b | 1530.78 | 1530.63 |
| ^{DTC}PMI | 1614.91 | 1615.68 |

The interactions of DTC-stapled PMI peptides with the p53-binding domains of MDM2 and MDMX were quantified using fluorescence polarization (FP) and surface plasmon resonance (SPR) techniques as described, and the Ki and Kd values are tabulated in Table 3 below.

TABLE 3
K_{d} and Kᵢ values of DTC-stapled PMI peptides for MDM2
and MDMX determined by SPR and FP techniques as well
as percent α-helix measured by CD spectroscopy

| | | | | | α- |
|---|---|---|---|---|---|
| | PMI-MDM2 | | PMI-MDMX | | helix |
| | Kᵢ (nM) | K_{d} (nM) | Kᵢ (nM) | K_{d} (nM) | (%) |
| PMI-0 | 5.9 ± 2.6 | 4.2 ± 0.90 | 5.2 ± 1.0 | 17 ± 1.2 | 0 |
| PMI(1,5)-a | 123 ± 28 | >500 | >1000 | >500 | 36 |
| PMI(1,5)-b | 51 ± 7.8 | 134 ± 5.5 | 39 ± 4.1 | 200 ±7.1 | 7.6 |
| PMI(2,6)-a | 4.5 ± 1.8 | 6.2 ± 0.70 | 4.4 ± 1.2 | 9.7 ± 1.2 | 5.9 |
| PMI(2,6)-b | 337± 136 | 121 ± 5.5 | 17 ± 1.2 | 48 ± 3.4 | 3.1 |
| PMI(4,8)-a | 2.2 ± 4.0 | 0.35 ± 0.12 | 1.9 ± 2.5 | 0.82 ± 0.70 | 25 |
| PMI(4,8)-b | 14 ± 1.5 | 20 ± 1.9 | 5.7 ± 1.7 | 12 ± 1.8 | 5.6 |
| PMI(5,9)-a | 29 ± 3.4 | 55 ± 3.3 | 24 ± 3.2 | 89 ± 5.6 | 0 |
| PMI(5,9)-b | 69 ± 13 | 90 ± 4.6 | 20 ± 2.7 | 54 ± 3.4 | 0 |
| PMI(8,12)-a | 1.7 ± 3.7 | 0.18 ± 0.19 | 3.3 ± 1.3 | 6.0 ± 0.90 | 33 |
| PMI(8,12)-b | 38 ± 6.5 | 57 ± 3.0 | 162 ± 31 | 400 ± 16 | 0 |
| ^{DTC}PMI | 2.1 ± 2.7 | 0.87 ± 0.49 | 2.0 ± 1.5 | 3.9 ± 2.0 | 76 |

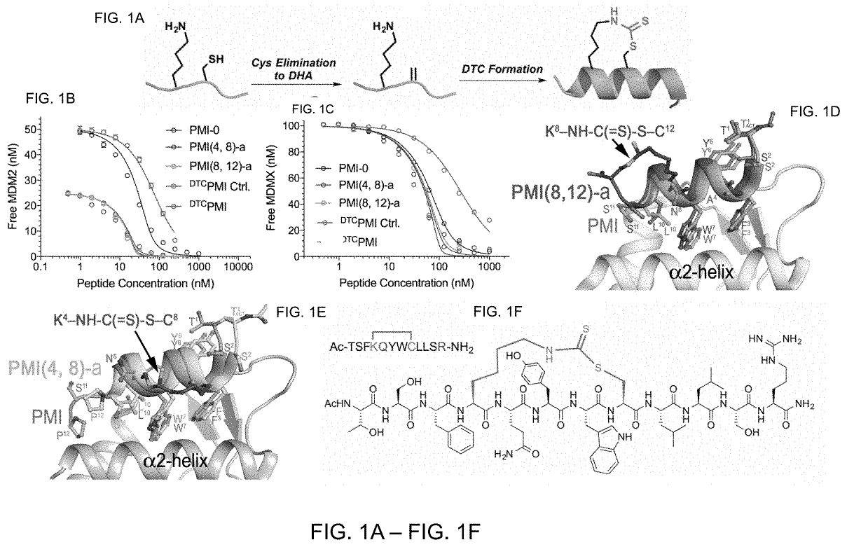

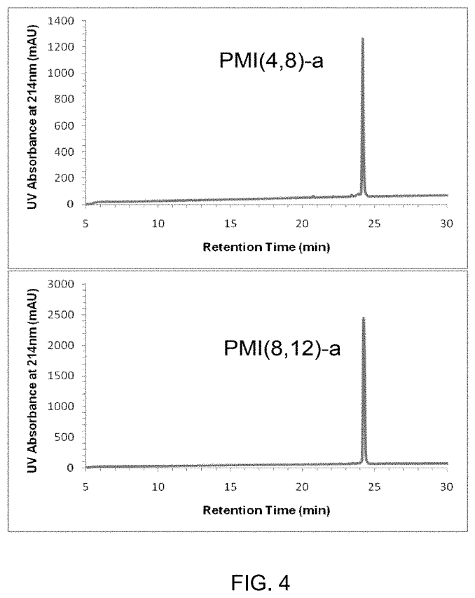

Compared with the N-acetylated and C-amidated wild-type peptide PMI-O, PMI(4,8)-a and PMI(8,12)-a bound more strongly to MDM2 and MDMX. In fact, the crosslinked Lys-Cys pair at positions (4, 8) enhanced peptide binding to both proteins by one order of magnitude as measured by SPR (FIGS. 1B-1C). Both PMI(4,8)-a and PMI(8,12)-a partially adopted an α-helical structure in aqueous solution (FIG. 5, Table 3). While not wanting to be bound by any particular theory, this result suggested that crosslinking Lys-Cys side chains stabilized peptide conformation productive for MDM2 and MDMX binding. Of note, the reversal of Lys-Cys (a) to Cys-Lys (b) in PMI was in general detrimental to peptide binding to MDM2 and MDMX (Table 3), indicating that the DTC crosslink is unidirectional functionally.

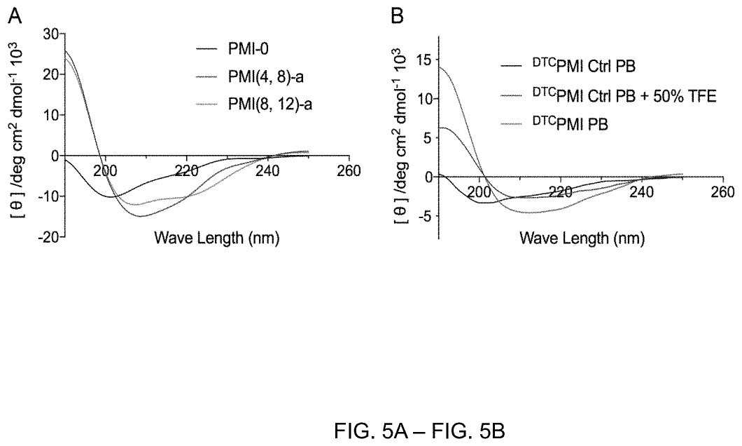

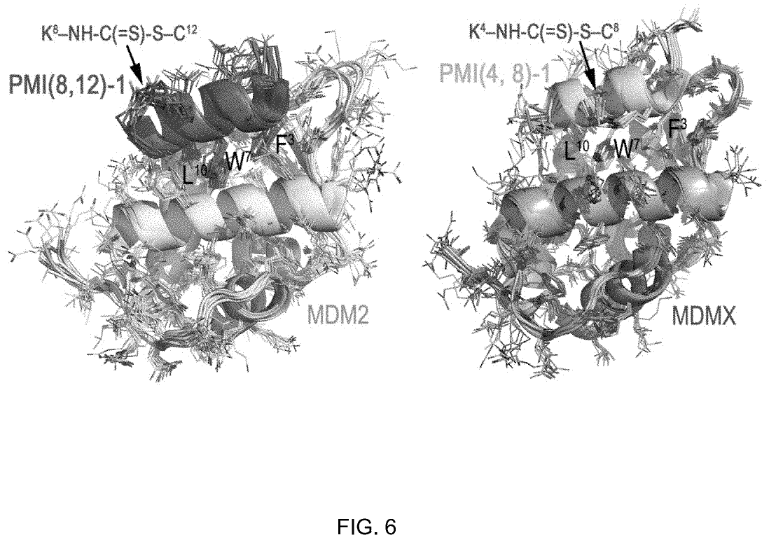

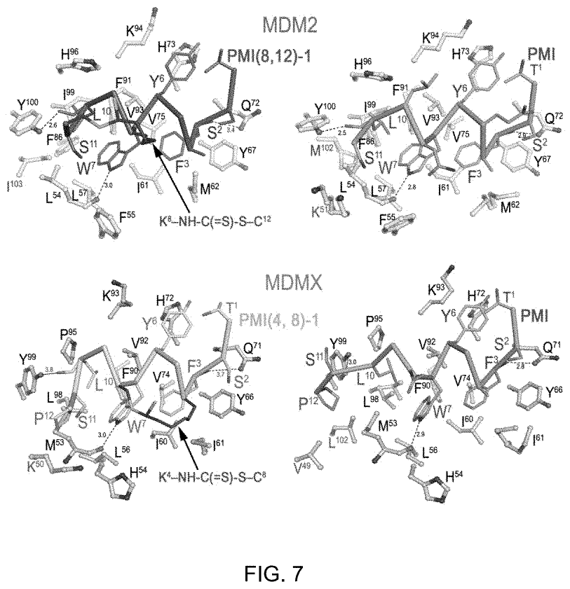

To structurally validate the DTC stapling chemistry, the co-crystal structures of MDM2-PMI(8,12)-a and MDMX-PMI(4,8)-a were solved at 1.8 and 2.7 Å resolution (Table 4), respectively, and compared them with the structures of MDM2 and MDMX in complex with PMI (FIGS. 1D-1E). Both complexes crystallized with multiple copies in the asymmetric unit of the crystal—12 for MDM2-PMI(8,12)-a and 8 for MDMX-PMI(4,8)-a (FIG. 6, Table 1). Whereas all 12 residues could be built into each PMI(8,12)-a peptide complexed with MDM2, PMI(4,8)-a was fully defined in only 3 copies of the MDMX complex with no density observed for Ser11 and/or Pro12 (FIG. 7). Alignment analysis of the PMI(8,12)-a conformation also indicated noticeable variability among the 12 copies of peptide (FIG. 8), as evidenced by the root-mean-square deviation (RMSD) between the main-chain atoms in the range of 0.48-1.35 Å (Table 4). In both complexes, however, the crystallographic density for all atoms of the crosslink formed between Lys (i) and Cys (i+4) unambiguously defined the geometry of the DTC staple (FIGS. 1D-1E). Tables 4 and 5 below show root mean square deviation (RMSD) between MDM2-PMI complexes and MDM2-PMI(8,12)-a (Table 4) or MDM2-PMI complexes (Table 5).

TABLE 4
The root mean square deviation (RMSD) between MDM2-PMI(8, 12)-a and MDM2-PMI complexes.

| | MDM2- | MDM2- | MDM2- | MDM2- | MDM2- | MDM2- | MDM2- | MDM2- |
|---|---|---|---|---|---|---|---|---|
| | PMI(8, 12)a | PMI(8, 12)b | PMI(8, 12)c | PMI(8, 12)d | PMI(8, 12)e | PMI(8, 12)f | PMI(8, 12)g | PMI(8, 12)h |
| MDM2- | — | 0.686 | 0.927 | 0.647 | 0.579 | 0.835 | 0.526 | 0.926 |
| PMI(8, 12)a | | | | | | | | |
| PMI(8, 12)a | — | 0.759 | 0.867 | 0.751 | 0.589 | 0.871 | 0.384 | 1.017 |
| MDM2-1 | — | 0.583 | 0.868 | 0.567 | 0.510 | 0.772 | 0.468 | 0.870 |
| MDM2- | 0.686 | — | 1.049 | 0.620 | 0.548 | 0.686 | 0.574 | 0.873 |
| PMI(8, 12)b | | | | | | | | |
| PMI(8, 12)b | 0.759 | — | 1.240 | 1.225 | 0.942 | 0.580 | 0.670 | 0.756 |
| MDM2b | 0.583 | — | 0.818 | 0.393 | 0.435 | 0.676 | 0.543 | 0.884 |
| MDM2- | 0.927 | 1.049 | — | 0.917 | 1.037 | 1.133 | 1.072 | 1.090 |
| PMI(8, 12)c | | | | | | | | |
| PMI(8, 12)c | 0.867 | 1.240 | — | 0.645 | 1.114 | 1.374 | 1.043 | 1.340 |
| MDM2c | 0.868 | 0.818 | — | 0.825 | 0.860 | 0.915 | 0.935 | 0.898 |
| MDM2- | 0.647 | 0.620 | 0.917 | — | 0.538 | 0.826 | 0.603 | 0.959 |
| PMI(8, 12)d | | | | | | | | |
| PMI(8, 12)d | 0.751 | 1.225 | 0.645 | — | 0.850 | 1.288 | 0.880 | 1.394 |
| MDM2d | 0.567 | 0.393 | 0.825 | — | 0.426 | 0.648 | 0.533 | 0.845 |
| MDM2- | 0.579 | 0.548 | 1.037 | 0.538 | — | 0.721 | 0.520 | 0.927 |
| PMI(8, 12)e | | | | | | | | |
| PMI(8, 12)e | 0.589 | 0.942 | 1.114 | 0.850 | — | 0.852 | 0.729 | 1.172 |
| MDM2e | 0.510 | 0.435 | 0.860 | 0.426 | — | 0.669 | 0.456 | 0.875 |
| MDM2- | 0.835 | 0.686 | 1.133 | 0.826 | 0.721 | — | 0.760 | 0.890 |
| PMI(8, 12)f | | | | | | | | |
| PMI(8, 12)f | 0.871 | 0.580 | 1.374 | 1.288 | 0.852 | — | 0.786 | 0.776 |
| MDM2f | 0.772 | 0.676 | 0.915 | 0.648 | 0.669 | — | 0.720 | 0.884 |
| MDM2- | 0.526 | 0.574 | 1.072 | 0.603 | 0.520 | 0.760 | — | 0.960 |
| PMI(8, 12)g | | | | | | | | |
| PMI(8, 12)g | 0.384 | 0.670 | 1.043 | 0.880 | 0.729 | 0.786 | — | 0.937 |
| MDM2g | 0.468 | 0.543 | 0.935 | 0.533 | 0.456 | 0.720 | — | 0.953 |
| MDM2- | 0.926 | 0.873 | 1.090 | 0.959 | 0.927 | 0.890 | 0.960 | — |
| PMI(8, 12)h | | | | | | | | |
| PMI(8, 12)h | 1.017 | 0.756 | 1.340 | 1.394 | 1.172 | 0.776 | 0.937 | — |
| MDM2h | 0.870 | 0.884 | 0.898 | 0.845 | 0.875 | 0.884 | 0.953 | — |
| MDM2- | 0.702 | 0.779 | 0.802 | 0.569 | 0.696 | 0.852 | 0.783 | 0.791 |
| PMI(8, 12)i | | | | | | | | |
| PMI(8, 12)i | 0.632 | 1.043 | 0.753 | 0.286 | 0.662 | 1.088 | 0.817 | 1.315 |
| MDM2i | 0.662 | 0.666 | 0.699 | 0.594 | 0.663 | 0.732 | 0.748 | 0.638 |
| MDM2- | 1.038 | 1.064 | 0.902 | 1.007 | 1.101 | 1.186 | 1.144 | 1.055 |
| PMI(8, 12)j | | | | | | | | |
| PMI(8, 12)j | 0.833 | 1.262 | 0.647 | 0.573 | 0.890 | 1.319 | 1.030 | 1.417 |
| MDM2j | 0.994 | 0.823 | 0.877 | 0.900 | 0.947 | 1.017 | 1.011 | 0.784 |
| MDM2- | 1.120 | 1.045 | 1.348 | 1.087 | 1.061 | 1.028 | 1.062 | 0.956 |
| PMI(8, 12)k | | | | | | | | |
| PMI(8, 12)k | 0.952 | 1.004 | 1.276 | 1.258 | 1.158 | 1.056 | 0.884 | 1.134 |
| MDM2k | 1.044 | 1.015 | 1.180 | 0.988 | 0.973 | 0.976 | 1.042 | 0.860 |

| | MDM2- | MDM2- | MDM2- | MDM2- | MDM2- |
|---|---|---|---|---|---|
| | PMI(8, 12)i | PMI(8, 12)j | PMI(8, 12)k | PMI(8, 12)l | PMI |
| MDM2- | 0.702 | 1.038 | 1.120 | 0.722 | 0.914 |
| PMI(8, 12)a | | | | | |
| PMI(8, 12)a | 0.632 | 0.833 | 0.952 | 0.853 | 0.731 |
| MDM2-1 | 0.662 | 0.994 | 1.044 | 0.650 | 0.885 |
| MDM2- | 0.779 | 1.064 | 1.045 | 0.752 | 0.754 |
| PMI(8, 12)b | | | | | |
| PMI(8, 12)b | 1.043 | 1.262 | 1.004 | 1.275 | 0.678 |
| MDM2b | 0.666 | 0.823 | 1.015 | 0.578 | 0.745 |
| MDM2- | 0.802 | 0.902 | 1.348 | 0.810 | 0.927 |
| PMI(8, 12)c | | | | | |
| PMI(8, 12)c | 0.753 | 0.647 | 1.276 | 0.661 | 0.897 |
| MDM2c | 0.699 | 0.877 | 1.180 | 0.713 | 0.851 |
| MDM2- | 0.569 | 1.007 | 1.087 | 0.520 | 0.783 |
| PMI(8, 12)d | | | | | |
| PMI(8, 12)d | 0.286 | 0.573 | 1.258 | 0.319 | 0.832 |
| MDM2d | 0.594 | 0.900 | 0.988 | 0.539 | 0.760 |
| MDM2- | 0.696 | 1.101 | 1.061 | 0.703 | 0.786 |
| PMI(8, 12)e | | | | | |
| PMI(8, 12)e | 0.662 | 0.890 | 1.158 | 0.989 | 0.643 |
| MDM2e | 0.663 | 0.947 | 0.973 | 0.603 | 0.782 |
| MDM2- | 0.852 | 1.186 | 1.028 | 0.866 | 0.770 |
| PMI(8, 12)f | | | | | |
| PMI(8, 12)f | 1.088 | 1.319 | 1.056 | 1.385 | 0.490 |
| MDM2f | 0.732 | 1.017 | 0.976 | 0.671 | 0.774 |
| MDM2- | 0.783 | 1.144 | 1.062 | 0.748 | 0.896 |
| PMI(8, 12)g | | | | | |
| PMI(8, 12)g | 0.817 | 1.030 | 0.884 | 0.974 | 0.620 |
| MDM2g | 0.748 | 1.011 | 1.042 | 0.688 | 0.912 |
| MDM2- | 0.791 | 1.055 | 0.956 | 0.877 | 0.818 |
| PMI(8, 12)h | | | | | |
| PMI(8, 12)h | 1.315 | 1.417 | 1.134 | 1.476 | 0.768 |
| MDM2h | 0.638 | 0.784 | 0.860 | 0.710 | 0.799 |
| MDM2- | — | 0.785 | 1.139 | 0.479 | 0.607 |
| PMI(8, 12)i | | | | | |
| PMI(8, 12)i | — | 0.501 | 1.337 | 0.314 | 0.560 |
| MDM2i | — | 0.600 | 1.013 | 0.494 | 0.594 |
| MDM2- | 0.785 | — | 1.299 | 0.850 | 0.878 |
| PMI(8, 12)j | | | | | |
| PMI(8, 12)j | 0.501 | — | 1.373 | 0.547 | 0.859 |
| MDM2j | 0.600 | — | 1.110 | 0.704 | 0.714 |
| MDM2- | 1.139 | 1.299 | — | 1.045 | 0.970 |
| PMI(8, 12)k | | | | | |
| PMI(8, 12)k | 1.337 | 1.373 | — | 1.340 | 0.829 |
| MDM2k | 1.013 | 1.110 | — | 0.923 | 0.984 |

Comparisons were made between 12 copies of MDM2-PMI(8, 12) complex (copies a, b, c, d, e, f, g, h, i, j, k, l) and one copy of MDM2-PMI complex.

TABLE 5
The root mean square deviation (RMSD) between MDMX-PMI(4, 8)-a and MDMX-PMI complexes.

| | MDMX- | MDMX- | MDMX- | MDMX- | MDMX- | MDMX- | MDMX- | MDMX- | MDMX- | MDMX- |
|---|---|---|---|---|---|---|---|---|---|---|
| | PMI(4, 8)a | PMI(4, 8)b | PMI(4, 8)c | PMI(4, 8)d | PMI(4, 8)e | PMI(4, 8)f | PMI(4, 8)g | PMI(4, 8)h | PMIa | PMIb |
| MDMX- | — | 0.976 | 0.600 | 0.677 | 0.936 | 0.762 | 0.586 | 0.589 | 1.254 | 1.270 |
| PMI(4, 8)a | | | | | | | | | | |
| PMI(4, 8)a | — | 2.188 | 1.011 | 1.091 | 1.815 | 1.100 | 0.721 | 0.274 | 1.059 | 1.081 |
| MDMXa | — | 0.464 | 0.479 | 0.538 | 0.461 | 0.641 | 0.529 | 0.594 | 0.966 | 0.969 |
| MDMX- | 0.976 | — | 0.618 | 0.545 | 0.660 | 0.830 | 0.724 | 0.611 | 1.534 | 1.574 |
| PMI(4, 8)b | | | | | | | | | | |
| PMI(4, 8)b | 2.188 | — | 1.018 | 0.752 | 1.089 | 1.002 | 1.411 | 0.590 | 2.461 | 2.582 |
| MDMXb | 0.464 | — | 0.505 | 0.454 | 0.504 | 0.735 | 0.526 | 0.583 | 0.853 | 0.855 |
| MDMX- | 0.600 | 0.618 | — | 0.684 | 0.498 | 0.761 | 0.599 | 0.672 | 1.096 | 1.096 |
| PMI(4, 8)c | | | | | | | | | | |
| PMI(4, 8)c | 1.011 | 1.018 | — | 1.029 | 0.946 | 1.010 | 1.003 | 1.060 | 0.902 | 0.902 |
| MDMXc | 0.479 | 0.505 | — | 0.575 | 0.368 | 0.683 | 0.491 | 0.575 | 0.939 | 0.940 |
| MDMX- | 0.677 | 0.545 | 0.684 | — | 0.543 | 0.756 | 0.647 | 0.627 | 1.106 | 1.107 |
| PMI(4, 8)d | | | | | | | | | | |
| PMI(4, 8)d | 1.091 | 0.752 | 1.029 | — | 0.732 | 0.464 | 1.157 | 0.354 | 1.318 | 1.319 |
| MDMXd | 0.538 | 0.454 | 0.575 | — | 0.490 | 0.744 | 0.471 | 0.632 | 0.845 | 0.847 |
| MDMX- | 0.936 | 0.660 | 0.498 | 0.543 | — | 0.733 | 0.711 | 0.564 | 1.508 | 1.541 |
| PMI(4, 8)e | | | | | | | | | | |
| PMI(4, 8)e | 1.815 | 1.089 | 0.946 | 0.732 | — | 1.036 | 1.338 | 0.585 | 2.112 | 2.198 |
| MDMXe | 0.461 | 0.504 | 0.368 | 0.490 | — | 0.617 | 0.463 | 0.552 | 0.896 | 0.899 |
| MDMX- | 0.762 | 0.830 | 0.761 | 0.756 | 0.733 | — | 0.816 | 0.746 | 1.388 | 1.389 |
| PMI(4, 8)f | | | | | | | | | | |
| PMI(4, 8)f | 1.100 | 1.002 | 1.010 | 0.464 | 1.036 | — | 0.945 | 0.319 | 1.104 | 1.105 |
| MDMXf | 0.641 | 0.735 | 0.683 | 0.744 | 0.617 | — | 0.710 | 0.774 | 1.113 | 1.114 |
| MDMX- | 0.586 | 0.724 | 0.599 | 0.647 | 0.711 | 0.816 | — | 0.501 | 1.036 | 1.037 |
| PMI(4, 8)g | | | | | | | | | | |
| PMI(4, 8)g | 0.721 | 1.411 | 1.003 | 1.157 | 1.338 | 0.945 | — | 0.366 | 0.453 | 0.456 |
| MDMXg | 0.529 | 0.526 | 0.491 | 0.471 | 0.463 | 0.710 | — | 0.496 | 0.914 | 0.916 |
| MDMX- | 0.589 | 0.611 | 0.672 | 0.627 | 0.564 | 0.746 | 0.501 | — | 1.066 | 1.067 |
| PMI(4, 8)h | | | | | | | | | | |
| PMI(4, 8)h | 0.274 | 0.590 | 1.060 | 0.354 | 0.585 | 0.319 | 0.366 | — | 0.362 | 0.364 |
| MDMXh | 0.594 | 0.583 | 0.575 | 0.632 | 0.552 | 0.774 | 0.496 | — | 0.955 | 0.956 |
| MDMX- | 1.254 | 1.534 | 1.096 | 1.106 | 1.508 | 1.388 | 1.036 | 1.066 | — | |
| PMIa | | | | | | | | | | |
| PMIa | 1.059 | 2.461 | 0.902 | 1.318 | 2.112 | 1.104 | 0.453 | 0.362 | — | 0.107 |
| MDMXa | 0.966 | 0.853 | 0.939 | 0.845 | 0.896 | 1.113 | 0.914 | 0.955 | — | 0.289 |
| | | | | | | | | | | 0.017 |
| MDMX- | 1.270 | 1.574 | 1.096 | 1.107 | 1.541 | 1.389 | 1.037 | 1.067 | 0.107 | — |
| PMIb | | | | | | | | | | |
| PMIb | 1.081 | 2.582 | 0.902 | 1.319 | 2.198 | 1.105 | 0.456 | 0.364 | 0.289 | — |
| MDMXb | 0.969 | 0.855 | 0.940 | 0.847 | 0.899 | 1.114 | 0.916 | 0.956 | 0.017 | — |

Comparisons were made between 8 copies of MDMX-PMI(4, 8) complex (copies a, b, c, d, e, f, g, h) and two copies of MDM-PMI complex (copies a, b).

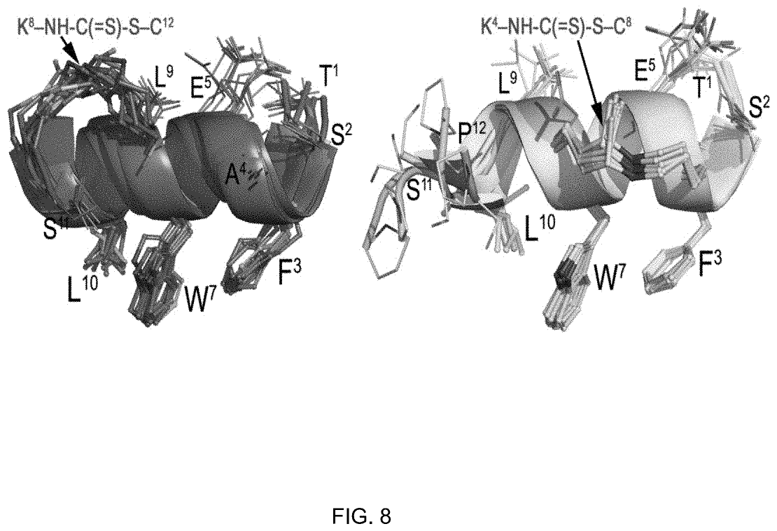

As shown in FIG. 1D, MDM2-bound PMI(8,12)-a largely overlapped with PMI, differing mainly in positions of the equivalent C_{α} atoms of residues Thr1-Trp7 with little change in the C-terminal region (Trp7-Ser11) (Table 4). More pronounced differences were observed between MDMX-bound PMI(4,8)-a and PMI (Table 5), with the backbone of the former longitudinally shifting ˜2 Å toward one side of the p53-binding pocket of MDMX and closer to its α2-helix in relation to PMI (FIG. 1E). This shift, while increasing PMI(4,8)-a contacts with the edge of the cavity formed by the α2-helix of MDMX, reduced hydrophobic contacts and lengthened some hydrogen bonds seen in the PMI-MDMX complex (FIG. 7). The DTC staple rigidified, at positions (8,12), the C-terminus of PMI in a helical conformation and extended, at positions (4,8), the C-terminal helix of PMI from Leu9 to Ser11 (FIGS. 1D-1E). The rigidity of PMI(8,12)-a or PMI(4,8)-a increased to such an extent that the local buried surface area (BSA) slightly decreased as compared with the BSA contributed by PMI to its interface with MDM2/MDMX (FIG. 9). While not wishing to be bound by any particular theory, this finding strongly suggests that DTC stapling-enhanced binding is energetically attributable to a reduced loss in entropy afforded by a pre-organized stable helix.

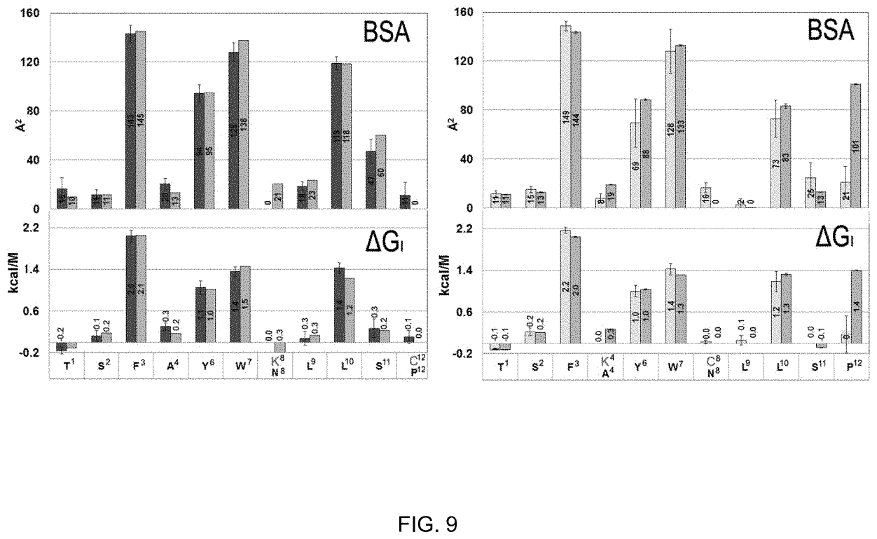

Side chain stapled peptides are structurally rigidified as compared with their linear counterparts and, thus, expected to be more resistant to proteolysis in vivo. HPLC and ESI-MS were used to evaluate the proteolytic stability of PMI(8,12)-a versus PMI-0 at 100 μM in cell culture medium in the presence of 25 μg/mL cathepsin G—an intracellular protease with dual specificities for both basic and bulky hydrophobic residues. As shown in FIG. 10, while PMI-0 was fully degraded by the enzyme within 30 min of co-incubation at room temperature, the DTC-stapled peptide was substantially more stable with a half-life of ˜8 h under identical conditions. Of note, the DTC structure is also stable in the presence of reduced glutathione (GST). When PMI(8,12)-a was incubated at 25° C. in PBS buffer with GST at 10 mM—a physiological concentration, no apparent breakdown of the DTC structure was observed over 24 h (FIG. 10).

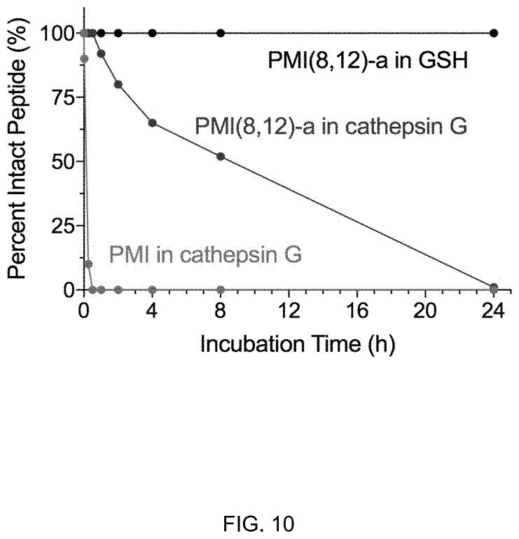

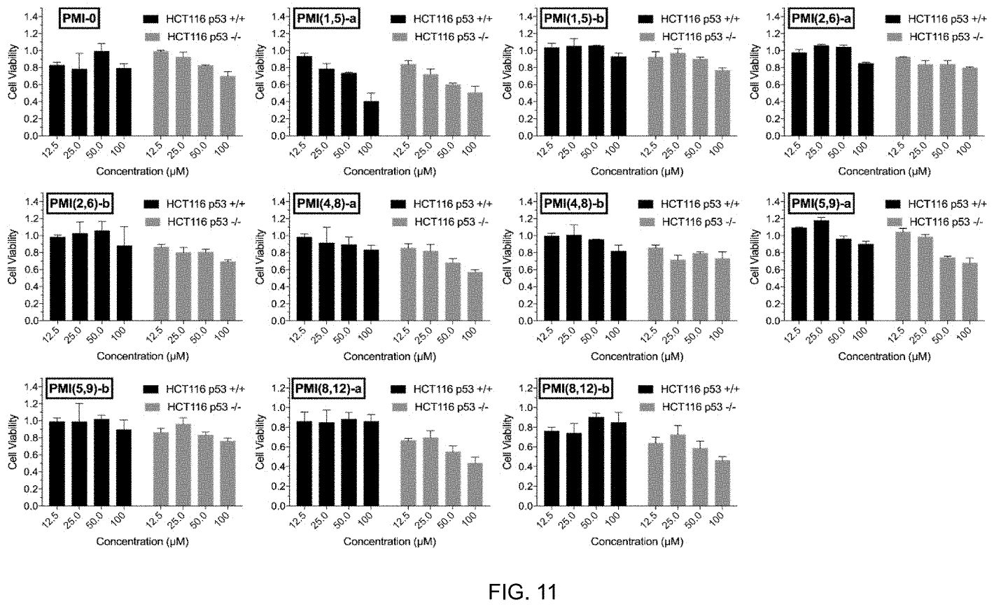

Verdine and colleagues have shown that structurally permissible stapling of a p53 peptide, while enhancing α-helicity and improving MDM2 binding, is not sufficient to endow the peptide with an ability to kill tumor cells. In fact, the amino acid composition of a stapled peptide, and cationicity in particular, is critical for its ability to traverse the cell membrane to exert biological activity. Not surprisingly, our DTC-stapled peptides showed little cytotoxicity against HCT116p53^{+/+} and HCT116p53^{-/-} cells at up to 100 μM as they all carried a net charge of either 0 or −1 (FIG. 11). Using PMI(4,8)-a as a template, two cationicity-enhancing mutations were made, E5Q and P12R, resulting in a DTC-stapled peptide termed ^{DTC}PMI with a +1 net charge (FIG. 1F). Compared with its unstapled control peptide, Ac-TSFKQYWCLLSR-NH₂, DTC crosslinking increased peptide binding affinity for MDM2 and MDMX by 50-fold as measured by SRP (FIGS. 1B-1C) or ˜20-fold by FP (FIG. 12), making ^{DTC}PMI one of the strongest dual-specificity peptide antagonists ever designed. Of note, ^{DTC}PMI also displayed a strong tendency to adopt α-helix on its own in aqueous solution (FIG. 5, Table 3), likely contributing energetically to its high-affinity binding to both MDM2 and MDMX.

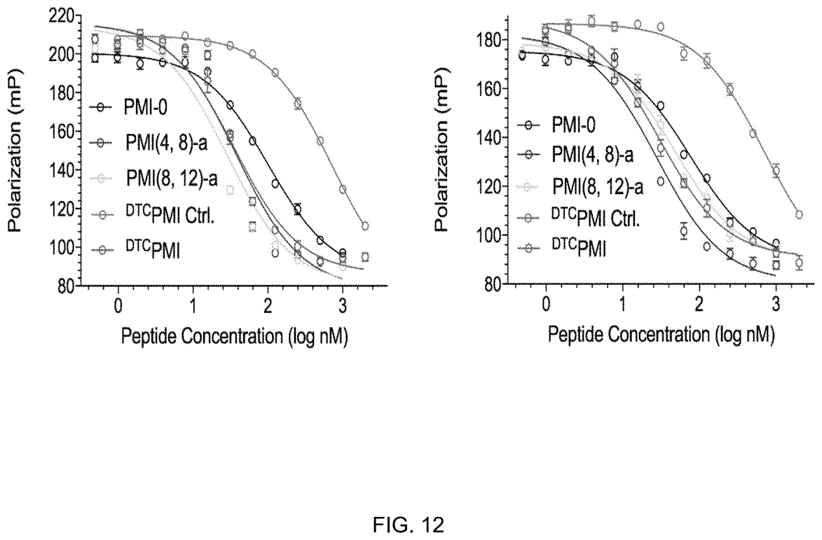

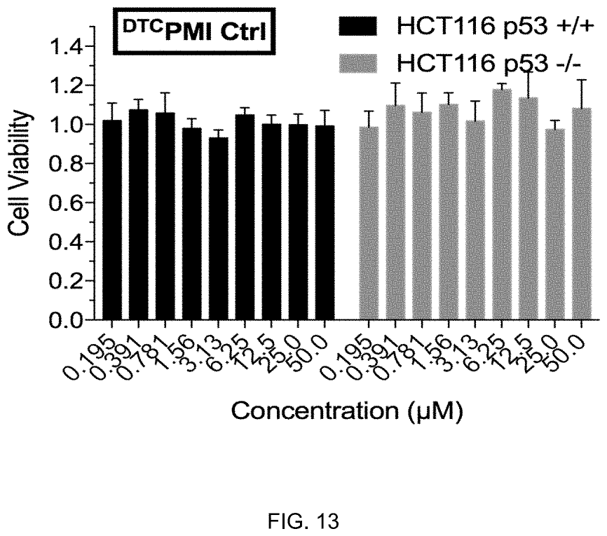

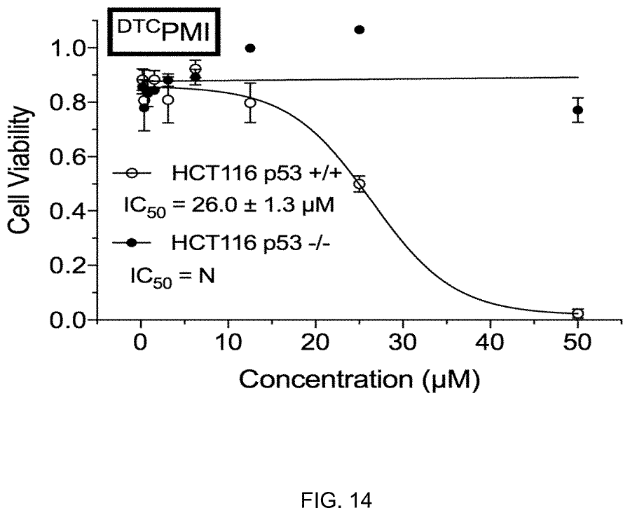

To functionally validate ^{DTC}PMI, it and its unstapled control were subjected to a cell viability assay using HCT116p5.3^{+/+} and p5.3″/− cells. While the control peptide exhibited no anti-proliferative activity against both cell lines at concentrations of up to 50 μM (FIG. 13), ^{DTC}PMI displayed p53-dependent growth inhibitory activity against HCT116p53^{+/+}, but not HCT116 p53^{-/-}, with an IC₅₀ value of ˜25 μM at 72 h (FIG. 1G and FIG. 14). To investigate the mechanisms of killing of HCT116p53^{+/+} by ^{DTC}PMI, the expression of MDM2, p53 and p21 were analysed by Western blotting. As shown in FIG. 1H, 8 h after treatment with ^{DTC}PMI, dose-dependent induction of p53, MDM2 and p21 became evident in HCT116 p53^{+/+} cells. Consistent with this result, dose-dependent induction of apoptosis of HCT116p53^{+/+} cells by ^{DTC}PMI was verified by fluorescence-activated cell sorting (FIG. 1I-1J and FIG. 15). Taken together, these findings support that ^{DTC}PMI actively traversed the cell membrane and killed tumor cells by antagonizing MDM2 to reactivate the p53 pathway.

In summary, a novel stapling strategy for peptide drug design has been developed by taking advantage of the DTC chemistry to crosslink the side chains of the two natural amino acid residues Lys and Cys at (i, i+4) positions. The DTC staple, structurally validated, induced the formation of and stabilized a productive α-helical conformation of PMI—a dual-specificity peptide antagonist of MDM2 and MDMX, enabling it to traverse the cell membrane and kill tumor cells by reactivating the p53 pathway. DTC stapling functionally rescued PMI that, on its own, failed to activate p53 in vitro and in vivo due to its poor membrane permeability and susceptibility to proteolytic degradation. Compared with other known stapling techniques, the solution-based DTC chemistry is simple, cost-effective, and highly efficient, promising an important new tool for peptide drug discovery and development for a variety of human diseases.

### Example 2: Dithiocarbamate-Inspired Side Chain Stapling Chemistry for Peptide Drug Design

Two major pharmacological hurdles severely limit the widespread use of small peptides as therapeutics: poor proteolytic stability and membrane permeability. Importantly, low aqueous solubility also impedes the development of peptides for clinical use. Various elaborate side chain stapling chemistries have been developed for α-helical peptides to circumvent this problem, with considerable success in spite of inevitable limitations. This Example describes a novel peptide stapling strategy based on the dithiocarbamate chemistry linking the side chains of residues Lys (i) and Cys (i+4) of unprotected peptides and apply it to a series of dodecameric peptide antagonist of the p53-inhibitory oncogenic proteins MDM2 and MDMX. Crystallographic studies of peptide-MDM2/MDMX complexes structurally validated the chemoselectivity of the dithiocarbamate staple bridging Lys and Cys at (i, i+4) positions. One dithiocarbamate-stapled PMI derivative, ^{DTC}PMI, showed a 50-fold stronger binding to MDM2 and MDMX than its linear counterpart. Importantly, in contrast to PMI and its linear derivatives, the ^{DTC}PMI peptide actively traversed the cell membrane and killed HCT116 tumor cells in vitro by activating the tumor suppressor protein p53. Compared with other known stapling techniques, this solution-based DTC stapling chemistry method is simple, cost-effective, regio-specific and environmentally friendly, promising an important new tool for the development of peptide therapeutics with improved pharmacological properties including aqueous solubility, proteolytic stability and membrane permeability.

Peptides are effective inhibitors of protein-protein interactions (PPI) and superior in many aspects as therapeutics to small molecule and protein drugs. However, peptides have two major pharmacological disadvantages—strong susceptibility to proteolytic degradation in vivo and poor membrane permeability, severely limiting their therapeutic efficacy. Importantly, another bottleneck in the development of peptides for clinical use is low solubility in aqueous solutions. Many therapeutic peptide drug candidates are abandoned because of their unacceptable solubility. For small peptides that adopt an α-helical structure upon interaction with target protein, various side chain stapling chemistries have been developed to improve their pharmacological properties via a pre-formed stable α-helix, among which the elaborate “hydrocarbon stapling” technique is probably best known. The hydrocarbon stapling chemistry takes advantage of Grubbs catalysts to crosslink on resin, via ruthenium-catalyzed olefin metathesis, two unnatural amino acids bearing olefinic side chains at (i, i+4) or (i, i+7) positions, and has been successfully used to design various peptide inhibitors with improved proteolytic stability, membrane permeability, and biological activity. One notable example is ALRN-6924, a hydrocarbon-stapled peptide antagonist of the oncogenic proteins MDM2 and MDMX that functionally inhibit the tumor suppressor protein p53. ALRN-6924, in phase 2 clinical trials for advanced solid tumors and lymphomas, kills tumor cells harboring wild-type p53 by antagonizing MDM2 and/or MDMX to reactivate the p53 pathway.

Despite its success in peptide drug design, hydrocarbon stapling can be technically cumbersome and costly due to the use of conformationally constrained unnatural amino acids and required transition metal carbene complexes as catalysts for olefin metathesis. Additionally, owing to an introduction of severely hydrophobic hydrocarbon stapling, another potential issue of this strategy is the problem of poor aqueous solubility, especially in those cases where the native hydrophilic side chains of Ser, Lys or Arg have to be sacrificed. To tackle these problems, described herein is a novel peptide stapling strategy by crosslinking the side chains of Lys and Cys at (i, i+4) positions via a thiocarbonyl group to form the dithiocarbamate (DTC) structure —NH—C(═S)—S—.

Materials. All reagents and solvents were purchased from Peptide International, Bachem Co. Ltd, Sigma or Millipore, and were purified when necessary.

Reversed phase analytical HPLC. Analytical HPLC was run on a SHIMADZU (Prominence LC-20AD) instrument using an analytical column (Dikma Tech “Diamonsil Plus C18”, 250×4.6 mM, 5 μm particle size, flow rate 1.0 mL/min, r.t.). Analytical injections were monitored at 214 nm. Solution A was 0.1% TFA in water, and solution B was 0.1% TFA in MeCN. Gradient A: A linear gradient of 10% to 10% B over 2 mins, then a linear gradient of 10% to 80% B over 25 mins.

High resolution mass spectra. HR-Q-TOF-MS was measured on an Agilent 6538 UHD Accurate Mass Q-TOF mass spectrometer.

Peptide and protein synthesis. All peptides and proteins used in this work were chemically synthesized, either in a stepwise fashion or via native chemical ligation. Peptides were synthesized using a machine-assisted Boc chemistry tailored from the optimized HBTU activation/DIEA in situ neutralization protocol. After chain assembly, side chain protecting groups were removed and peptides cleaved from the resin by treatment with anhydrous HF and p-cresol (9:1) at 0° C. for 1 h. Crude peptides were precipitated with cold ether and purified by preparative C18 reversed-phase (RP) HPLC. The synthesis of ²⁵⁻¹⁰⁹MDM2 and ²⁴⁻¹⁰⁸MDMX was described previously, and obtained via native chemical ligation. The reaction between MDM2(25-76)-COSR and MDM2(77-109) (1.5 eq) or between MDMX (24-75)-COSR and MDMX (76-108) (1.5 eq) was carried out at a total peptide concentration of 10-20 mg/ml in 0.25 M phosphate buffer (pH 7.1) containing 6 M guanidine hydrochloride, 50 mM MPAA and 20 mM TCEP.HCl. They went to completion overnight as monitored by analytical HPLC. The ligation products were purified by preparative RP-HPLC to homogeneity. The molecular masses of all peptides and proteins were ascertained by electrospray ionization mass spectrometry.

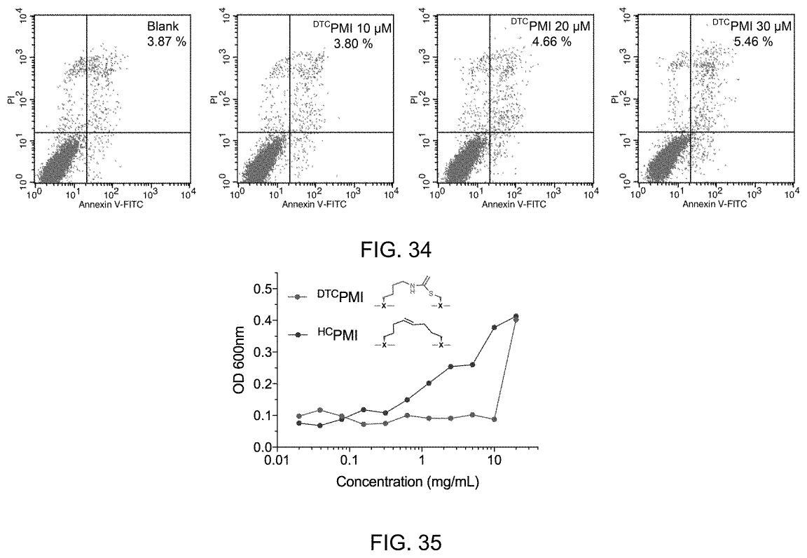

Synthesis of Stapled PMIs. PMI(1,5)-a is used as an example (FIG. 35). Cys to Dha. Buffer A containing 6 M guanidine hydrochloride and 100 mM Na₂HPO₄, pH=8.5, and Buffer B containing 6 M guanidine hydrochloride and 100 mM NaH₂PO₄, pH=2.5, were prepared prior to the reaction. 3 mL Buffer B was used for dissolving 50 mg PMI(1K,5C) for storage. 75 mg Bisamide reagent (1.5 mg per linear peptide) was dissolved in 47 mL Buffer A, followed by a slow addition of Buffer B containing the linear peptide. The reaction was stirred at room temperature overnight and monitored by analytical HPLC. The crude intermediate product PMI(1K,5DHA) was purified by preparative RP-HPLC to homogeneity (35 mg). DTC cyclization. 20 mg PMI(1K,5DHA) was dissolved in 10 mL ethanol, followed by addition of 1 mL Et₃N and 1 mL CS₂. The reaction proceeded with stirring overnight at room temperature until a complete conversion. After the solvent was removed, the residual material was purified by preparative RP-HPLC to yield the stapled product PMI(1,5)-a (10 mg).

Surface plasmon resonance (SPR). Competition binding kinetics was carried out at 25° C. using a Biacore T100 SPR instrument and ¹⁵⁻²⁹p53-immobilized CM5 sensor chips as described. ²⁵⁻¹⁰⁹MDM2 and ²⁴⁻¹⁰⁸MDMX at 50 nM or 100 nM were incubated in 10 mM HEPES buffer containing 150 mM NaCl, 0.005% surfactant P20, pH 7.4, with varying concentrations of peptide inhibitor before SPR analysis. The concentration of unbound MDM2 or MDMX in solution was deduced, based on p53-association RU values, from a calibration curve established by RU measurements of different concentrations of MDM2/MDMX injected alone. Two replicates and three independent experiments were performed.

Fluorescence polarization (FP). A FP-based competitive binding assay was established using ²⁵⁻¹⁰⁹MDM2, ²⁴⁻¹⁰⁸MDMX and a fluorescently tagged PMI peptide as previously described. Succinimidyl ester-activated carboxytetramethylrhodamine (TAMRA-NHS) was covalently conjugated to the N-terminus of PMI (TSFAEYWNLLSP) (K_{d}^{PMI-MDM2}=3.2 nM, K_{d}^{PMI-MDMX}=8.5 nM). Unlabeled PMI competed with TAMRA-PMI for MDM2/MDMX binding, based on which the K_{d} values of TAMRA-PMI with MDM2 and MDMX were determined by changes in FP to be 0.62 and 0.72 nM, respectively. As an additional positive control, the binding of Nutlin-3 to MDM2 and MDMX was quantified, yielding respective Ki values of 5.1 nM and 1.54 μM, similar to the values reported in the literature. For dose-dependent competitive binding experiments, MDM2 or MDMX protein (50 nM) was first incubated with TAMRA-PMI peptide (10 nM) in PBS (pH 7.4) on a Costar 96-well plate, to which a serially diluted solution of test peptide was added to a final volume of 125 μL. After 30 min of incubation at room temperature, the FP values were measured at λₑₓ=530 nm and λₑₘ=580 nm on a Tecan Infinite M1000 plate reader. Curve fitting was performed using GRAPHPAD PRISM software, and Ki values were calculated as described previously. Two replicates and three independent experiments were performed.

Cell Viability Assay. The human colon cancer cell lines HCT116 p53^{+/+} and HCT116 p53^{-/-} were maintained in McCoy's 5 A medium (Invitrogen) supplemented with 10% heat-inactivated FCS and 1% penicillin-streptomycin at 37° C. with 5% CO₂ under fully humidified conditions. Cells (3×10³ cells/well) were seeded at in 96-well plates and treated with PMI and stapled PMIs at various concentrations in serum-free media for 8 hours, followed by serum complementary and additional incubation for 64 hours. The absorbance at 450 nm was then measured followed by the addition of CCK8 kit, and percent cell viability was calculated on the ratio of the A₄₅₀ of sample wells versus reference wells.

Circular Dichroism (CD) Spectroscopy. Compounds were dissolved in PB (pH=7.2) to concentrations ranging from 10-50 μM. The spectra were obtained on a Jasco J-715 spectropolarimeter at 20° C. The spectra were collected using a 0.1 cm path-length quartz cuvette with the following measurement parameters: wavelength, 185-255 nm; step resolution 0.1 nm; speed, 20 nm min⁻¹; accumulations, 6; bandwidth, 1 nm. The helical content of each peptide was calculated as reported previously.

Proteolytic Stability. PMI-0 and the stapled peptide PMI(8,12)-a were incubated at 100 μM each in RPMI 1640 with 25 μg/ml cathepsin G—an intracellular protease with dual specificities for both basic and bulky hydrophobic residues. RP-HPLC was used to monitor and quantify time-dependent peptide hydrolysis.

Stability in GSH. PMI(8,12)-a was incubated at 25° C. in PBS buffer with reduced glutathione at 10 mM. RP-HPLC and ESI-MS were used to monitor and quantify time-dependent breakdown of the DTC staple.

Cellular uptake of ^{DTC}PMI. HCT116 p53^{+/+} cells were seeded in four-well chambered cover-glass (6×10⁴ cells per well) and allowed to grow overnight. Cells were then incubated with 20 μM FITC-^{DTC}PMI Ctrl. or FITC-^{DTC}PMI for 4 h. Cells were washed with Dulbecco's phosphate buffered saline, fixed with 4% (wt/vol) paraformaldehyde, finally incubated by DAPI to stain the cell nucleus. Imaged using an LSM 510 Zeiss Axiovert 200M (v4.0) confocal microscope. Images were analyzed using an LSM image browser.

Western Blot Analysis. HCT116 p53^{+/+} cells (1×10⁶) incubated at 37° C. were treated with ^{DTC}PMI (10, 20, 30 μM) in serum-free media for 8 hours. The cells were lysed (20 mM Tris-HCl pH 8.0, 0.8% SDS, 1 mM PMSF, 1 U mL⁻¹ benzonase nuclease) and the crude lysates were clarified by brief centrifugation and total protein concentration was determined by using the Pierce BCA protein assay. Aliquots containing 5 μg of total protein were run on 4-12% Bis-Tris polyacrylamide gels (Invitrogen). Proteins were detected by chemiluminescence reagent (Perkin Elmer) using antibodies specific for p53 (Santa Cruz Biotechnology), MDM2 (Santa Cruz Biotechnology), p21 (Merck Millipore), and β-actin (Sigma-Aldrich).

Cell Apoptosis Assay. HCT116 p53^{+/+} or HCT116 p53^{-/-} cells were seeded in 6-well tissue culture plates (3×10⁵ cells per well) for 12 h and treated with 10, 20, or 30 μM ^{DTC}PMI in serum-free media for 8 hours, followed by serum complementary and additional incubation for 40 hours. No treatment controls were established. Culture medium that may contain detached cells was collected, and attached cells were trypsinized. After centrifugation and removal of the supernatants, cells were resuspended in 300 μL of 1×binding buffer which was then added to 5 μL of annexin V-FITC and incubated at room temperature for 15 min. After addition of 10 μL of PI, the cells were incubated at room temperature for another 10 min in the dark. The stained cells were analyzed by a flow cytometer (BD-FACSVerse).

All Hydrocarbon Stapling. 400 mg Rink Amide MBHA resin was swelled with DCM (5 mL) for 20 mins. Then the resin was treated with 20% piperidine/DMF twice (10 and 5 mins), followed by washing with DMF (5 times), DCM (5 times) and DMF (5 times). For coupling of the first amino acid, Fmoc-AA-OH (1 mmol), HCTU (0.9 mmol), DIEA (2 mmol) and DMF (6 mL) were mixed for 2 mins and then added to the resin. After 2 hrs, the resin was washed with DMF (5 times), DCM (5 times), and DMF (5 times). The peptide couplings of N-Fmoc-α-pentene amino acid S₅ were carried out over a single two hours coupling cycle using 2 eq. of the Fmoc protected amino acids. The deprotection, washing, coupling and washing steps were repeated until all the amino acid residues were assembled reagent. The peptide-bound resin was treated with 20% piperidine/DMF to remove the Fmoc group from the N-terminus. After the resin was washed it was treated with 3 mL solution of acetic anhydride and pyridine (1:1) for 20 mins. Then the resin was washed with DMF (5 times), DCM (5 times), and DMF (5 times). The ring-closing metathesis reaction was carried out in 1,2-dichloroethane (DCE) at room temperature (20-25° C.) using Grubbs' first-generation catalyst (10 mM). After the first round of the 2 hrs metathesis, w the same procedure was repeated for a second round of catalyst treatment with fresh catalyst solution, then the peptide-resin was washed with DMF (5 times), DCM (5 times). Peptides were cleaved from their resin by treatment with reagent K (80% TFA, 5%, H₂O, 2.5% EDT, 5% Thioanisole and 7.5% phenol) for 4 hrs at room temperature. After completion of the cleavage reaction, TFA was evaporated by blowing with Ar. The crude peptides were obtained by precipitation with 40 mL of cold diethyl ether and purified with preparative RP-HPLC to yield the stapled product (^{HC}PMI).

Comparison of Solubility between DTC and Hydrocarbon link. 1 mg each of ^{DTC}PMI and ^{HC}PMI were mixed in 50 μL PBS Buffer individually, and then transferred 40 μL the suspension to the Costar mini 96-well plate. The suspension was gradient diluted from 20 mg/mL to 0.0195 mg/mL. OD values were measured at 600 nm on a Biotech Synergy 4 plate reader. PBS was set as blank control.

Crystallization of stapled-PMI complexes. Initial screening for crystals was done with an Art Robbinson crystallization robot using vapor diffusion sitting trials of sparse matrix crystallization screens: the Hampton crystal screen I and II (Hampton Research), the precipitant wizard screen (Emerald BioSystems), the synergy screen (Emerald BioSystems) and the ProComplex and MacroSol screens from Molecular Dimensions. All crystallization experiments were performed with complexes at 8-10 mg/ml in 20 mM Tris pH 7.4. Conditions that produced micro crystals were then reproduced and optimized using the hanging-drop vapor diffusion method (drops of 0.5 μl of protein and 0.5 μl of precipitant solution equilibrated against 700 μl of reservoir solution). Diffraction quality crystals for MDM2-PMI(8,12)-a complex were obtained from a solution containing 1.34 M ammonium sulfate, 6.7% (v/v) glycerol, 50 mM magnesium sulfate, and 0.1 M imidazole pH 6.5. Prior to being frozen, the crystals were transferred into the crystal growth solution supplemented with 20% (v/v) 2-methyl-2,4-pentanediol (MPD). Crystals of MDMX-PMI(4,8)-a complex were grown from 30% (v/v) 2-propanol, 30% (v/v) PEG 3350, and 0.1 M Tris-HCl pH 8.5 and frozen from the same solution supplemented with 20% (v/v) MPD.

Data Collection, Structure Solution and Refinement. Diffraction data for both complexes were collected at the Stanford Synchrotron Radiation Light Source (SSRL) BL12-2 beam line equipped with Pilatus 6M PAD area detector. The MDM2-PMI(8,12)-a complex crystals belong to a space group C222₁ with unit-cell parameters a=90.8 Å, b=157.5 Å, and c=196.7 Å with twelve complexes copies present in the asymmetric unit. The MDMX-PMI(4,8)-a complex crystals belong to a space group P1 with unit-cell parameters a=43.3 Å, b=47.7 Å, c=93.4 Å, α=76.7°, β=89.9°, and γ=72.6° and eight complexes in the asymmetric unit (Table 9). The data for both the complexes were processed and scaled with HKL2000. Structures were solved by molecular replacement with Phaser from the CCP4 program suite based on the coordinates extracted from the structure of MDM2-PMI complex (PDB code: 3EQS) and MDMX-PMI complex (PDB code: 3EQY). The models were refined using Refmac and the structure manually rebuilt with COOT. The MDM2-PMI(8,12)-a complex was refined to R_{factor} of 0.197 and R_{free} of 0.245. The MDMX-PMI(4,8)-a complex was refined to R_{factor} of 0.278 and R_{free} of 0.336. 97.8% and 98.8% of residues fell within allowed regions of the Ramachandran plot as determined by MolProbity, respectively (Table 9).

### Results and Discussion

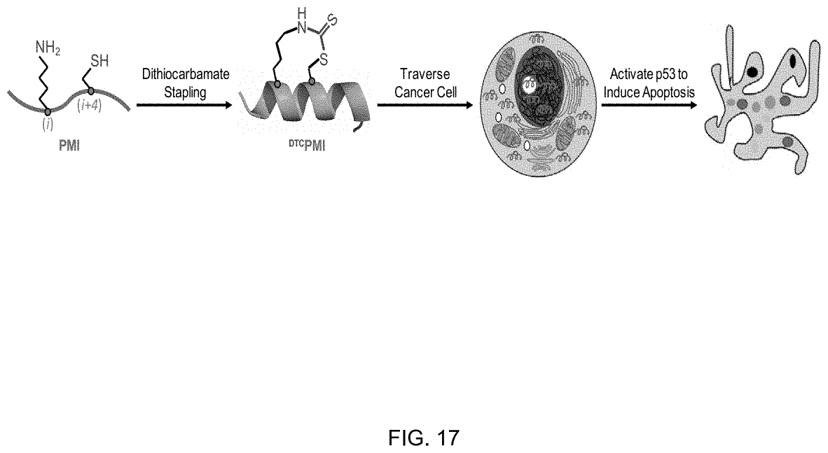

This solution chemistry for unprotected peptides entails the conversion of Cys via oxidative elimination to dehydroalanine (DHA), which subsequently reacts with the ε-amino group of Lys in the presence of carbon disulfide (CS₂) (FIG. 18A). In this proof-of-concept study, PMI was used—a potent dodecameric peptide antagonist of MDM2 and MDMX that, despite its high affinity for both proteins, fails to activate p53 and kill p5.3^{+/+} tumor cells due presumably to its inability to traverse the cell membrane and susceptibility to proteolytic degradation.

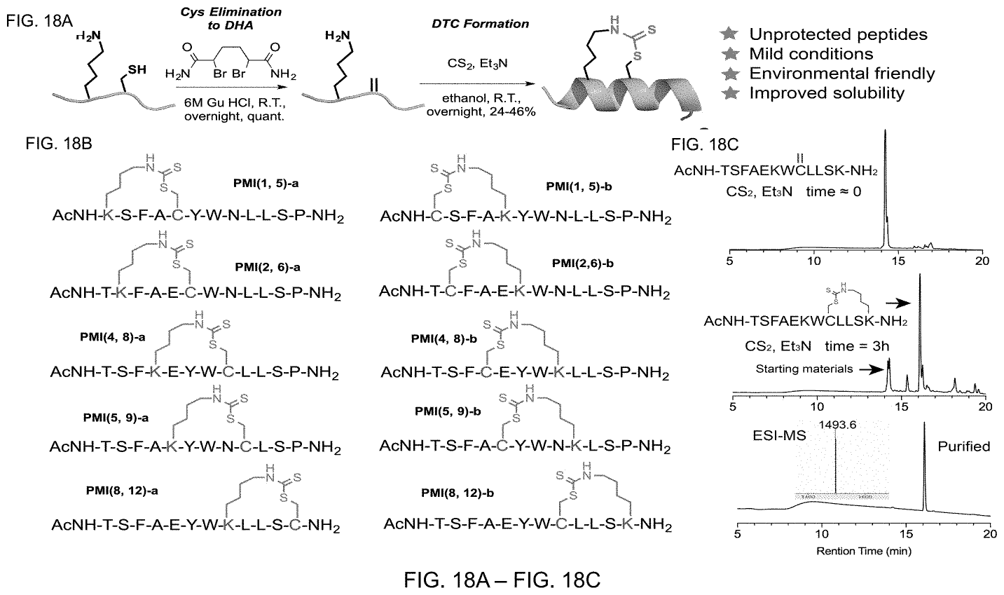

Previous structural and functional studies of PMI (TSFAEYWNLLSP) identified Phe3, Trp7 and Leu10 as the most critical residues for MDM2/MDMX binding. Thus, those three residues were maintained in the design of DTC-stapled peptides and Lys-Cys (a) or Cys-Lys (b) pairs were introduced into (1, 5), (2, 6), (4, 8), (5, 9), or (8, 12) positions of PMI (FIG. 18B). These N-acetylated and C-amidated peptides were synthesized using solid phase peptide synthesis, and purified by HPLC to homogeneity. Conversion of Cys to DHA, monitored by HPLC and electrospray ionization mass spectrometry (ESI-MS), was achieved in an overnight reaction in 6 M GuHCl, pH 8.0, in the presence of the bisamide of the 1,4-dibromobutane core, to give the elimination-prone sulfonium salt, followed by HPLC purification. Crosslinking DHA and Lys side chains was readily accomplished overnight in ethanol containing Et₃N and CS₂ (FIG. 18A, FIG. 2), as verified by ESI-MS (FIG. 19A-FIG. 19C and Table 6 below), resulting in 10 DTC-stapled constructs termed PMI(1,5)-a, PMI(1,5)-b, PMI(2,6)-a, PMI(2,6)-b, PMI(4,8)-a, PMI(4,8)-b, PMI(5,9)-a, PMI(5,9)-b, PMI(8,12)-a and PMI(8,12)-b (FIG. 18B).

TABLE 6
Yield and HR-MS spectrometry data
for DTC-stapled PMI peptides.

| | | Calculated Mass | | |
|---|---|---|---|---|
| Compound | Yield (%) | (M/2 + H) | Found | Method |
| PMI(1, 5)-a | 28 | 756.3441 | 756.3483 | Q-TOF |
| PMI(1, 5)-b | 37 | 756.3441 | 756.3451 | Q-TOF |
| PMI(2, 6)-a | 34 | 746.3416 | 746.3412 | Q-TOF |
| PMI(2, 6)-b | 39 | 746.3416 | 746.3419 | Q-TOF |
| PMI(4, 8)-a | 42 | 778.8493 | 778.8498 | Q-TOF |
| PMI(4, 8)-b | 33 | 778.8493 | 778.8499 | Q-TOF |
| PMI(5, 9)-a | 24 | 750.3259 | 750.3294 | Q-TOF |
| PMI(5, 9)-b | 27 | 750.3259 | 750.3271 | Q-TOF |
| PMI(8, 12)-a | 45 | 765.8414 | 765.8433 | Q-TOF |
| PMI(8, 12)-b | 38 | 765.8414 | 765.8416 | Q-TOF |
| ^{DTC}PMI | 46 | 807.8814 | 807.8834 | Q-TOF |

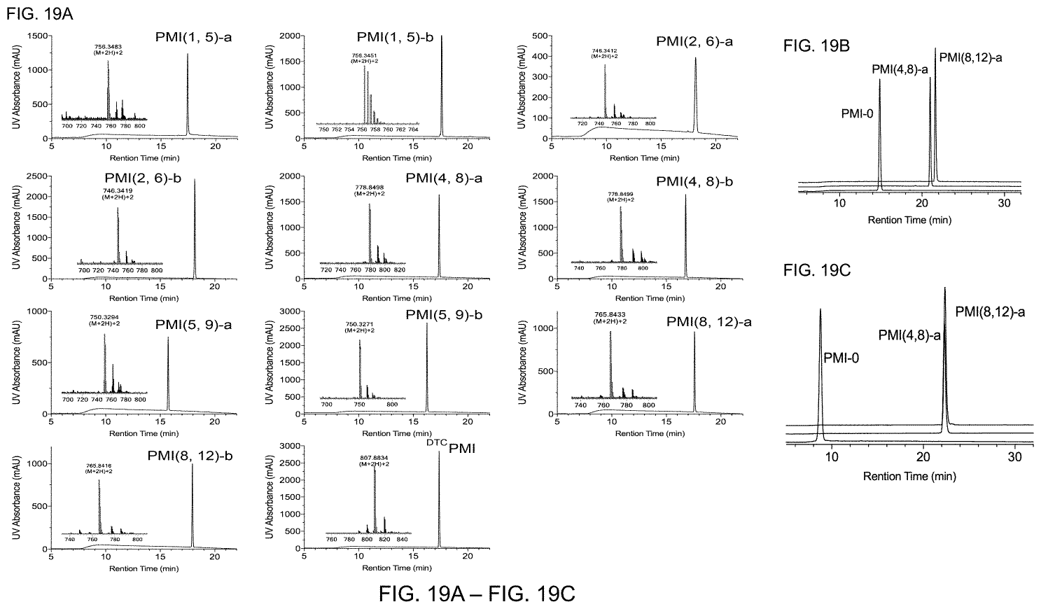

Although this Example focused on PMI and its derivatives, the DTC stapling chemistry is expected to be applicable to other peptide systems as well. The transactivation domain (TAD) of p53, a peptide of 12-15 amino acid residues, has been extensively studied for its interaction with MDM2 and MDMX. Ser20 was mutated to Cys of a TAD peptide of p53, i.e., 16-27p53 (QETFSDLWKLLP), and stapled through a DTC linkage between Cys20 and Lys24. (FIG. 20A-FIG. 20E). Importantly, when Lys24 was replaced by Ornithine, diaminobutyric acid or diaminopropionic acid, the DTC staple failed to form under otherwise identical experimental conditions. While not wishing to be bound by any particular theory, this result suggests that the side chains of Cys and Lys (or Lys and Cys) at (i, i+4) positions are well-paired geometrically for the DTC chemistry.

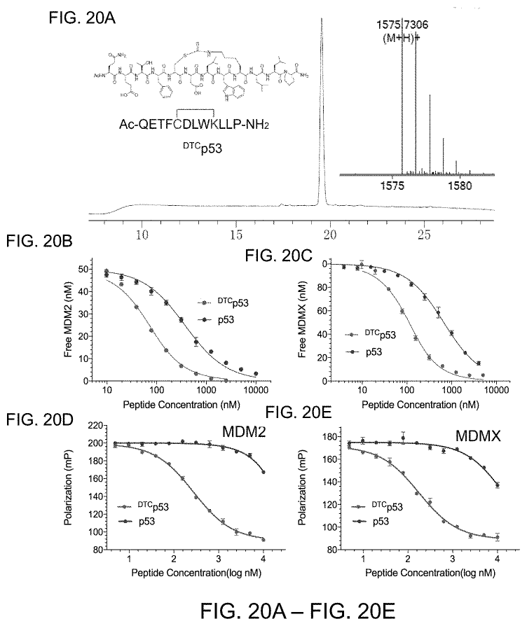

To furthermore demonstrate the regio-selectivity of the DTC chemistry, the PMI-derived peptide Ac-TSFAEKWCLLSK-NH₂ was examined, where Cys and two Lys residues are present in the same sequence. The question was: can Cys form two competing DTC staples with the two Lys residues in the same sequence, at (i, i+4) and (i, i+2) positions? Only one predominant reaction product containing a DTC staple was recovered (FIG. 18C). After HPLC purification, the product was subjected to tryptic digestion and mass spec analysis, and the data unambiguously demonstrated that the DTC staple had formed between Cys and Lys at (i, i+4) positions, but not at (i, i+2) positions (FIG. 21).

Formation of the DTC crosslink between Lys and Cys side chains appears stereo-selective despite that possibility that Michael addition of Lys-NH—C(═S)S⁻ (product of the reaction between the amino group —NH₂ and CS₂) to dehydro-alanine could yield two epimeric compounds (L-Cys and R-Cys) in equal quantities. In reality, however, one predominant isomer was identified and purified by HPLC for subsequent characterization (FIG. 18C, FIG. 19A-FIG. 19C), while a very minor isomer of an identical molecular mass was chromatographically resolved but discarded. To ascertain the purity of DTC-stapled peptides, PMI(4,8)-a and PMI(8,12)-a were analysed on HPLC at different gradients. Both PMI(4,8)-a and PMI(8,12)-a, along with the wild type control peptide PMI-0, eluted as single and symmetric peaks at 30-60% and 35-45% acetonitrile over 30 min (FIG. 19A-FIG. 19C).

The influence of DTC staple on binding affinities of peptides with target proteins was next evaluated. The interactions of DTC-stapled PMI peptides with the p53-binding domains of MDM2 and MDMX were quantified using fluorescence polarization (FP) and surface plasmon resonance (SPR) techniques, and the Kᵢ and K_{d} values are tabulated in Table 7.

TABLE 7
K_{d} and Kᵢ values of DTC-stapled peptides for MDM2
and MDMX determined by SPR and FP techniques as
well as percent α-helix measured by CD spectroscopy

| | | | | | α- |
|---|---|---|---|---|---|
| | PMI-MDM2 | | PMI-MDMX | | helix |
| | Kᵢ (nM) | K_{d} (nM) | Kᵢ (nM) | K_{d} (nM) | (%) |
| PMI-0 | 5.9 ± 2.6 | 4.2 ± 0.90 | 5.2 ± 1.0 | 17 ± 1.2 | 9.77 |
| PMI(1,5)-a | 123 ± 28 | >500 | >1000 | >500 | 6.50 |
| PMI(1,5)-b | 51 ± 7.8 | 134 ± 5.5 | 39 ± 4.1 | 200 ± 7.1 | 8.38 |
| PMI(2,6)-a | 4.5 ± 1.8 | 6.2 ± 0.70 | 4.4 ± 1.2 | 9.7 ± 1.2 | 15.3 |
| PMI(2,6)-b | 337 ± 136 | 121 ± 5.5 | 17 ± 1.2 | 48 ± 3.4 | 4.81 |
| PMI(4,8)-a | 2.2 ± 4.0 | 0.35 ± 0.12 | 1.9 ± 2.5 | 0.82 ± 0.70 | 39.3 |
| PMI(4,8)-b | 14 ± 1.5 | 20 ± 1.9 | 5.7 ± 1.7 | 12 ± 1.8 | 9.15 |
| PMI(5,9)-a | 29 ± 3.4 | 55 ± 3.3 | 24 ± 3.2 | 89 ± 5.6 | 6.88 |
| PMI(5,9)-b | 69 ± 13 | 90 ± 4.6 | 20 ± 2.7 | 54 ± 3.4 | 9.58 |
| PMI(8,12)-a | 1.7 ± 3.7 | 0.18 ± 0.19 | 3.3 ± 1.3 | 6.0 ± 0.90 | 43.3 |
| PMI(8,12)-b | 38 ± 6.5 | 57 ± 3.0 | 162 ± 31 | 400 ± 16 | 0.23 |
| ^{DTC}PMI Ctrl. | 42 ± 4.0 | 47 ± 3.0 | 47 ± 3.1 | 220 ± 11 | 16.6 |
| ^{DTC}PMI | 2.1 ± 2.7 | 0.87 ± 0.49 | 2.0 ± 1.5 | 3.9 ± 2.0 | 62.2 |
| p53 | >1000 | 346 ± 19 | 987 ± 17 | 614 ± 26 | N/A |
| ^{DTC}p53 | 16 ± 1.2 | 46 ± 2.7 | 12 ± 1.3 | 62 ± 4.9 | N/A |

Note:
In the SPR-based quantification method, where direct binding of stapled peptide to MDM2/MDMX was measured, Kd (the equilibrium dissociation constant) values were given by a non-linear regression analysis using the equation Kd = [peptide][MDM2/MDMX]/[complex]. In the FP-based competitive binding assay, where a fluorescently tagged PMI peptide in complex with MDM2/MDMX was competed off by stapled peptide, Ki (equilibrium inhibition constant) values were calculated using the equation Ki = [I]₅₀/([L]₅₀/Kd + [P]₀/Kd + 1) (see Nikolovska-Coleska Z, Wang S, et al. Anal Biochem. 2004, 332: 261-73, which is incorporated by reference herein in its entirety), in which [I]₅₀ denotes the concentration of stapled peptide at 50% inhibition, [L]₅₀ the concentration of labeled PMI at 50% inhibition, [P]₀ the concentration of free MDM2/MDMX at 0% inhibition, and Kd the equilibrium dissociation constant of the MDM2/MDMX-PMI complex.

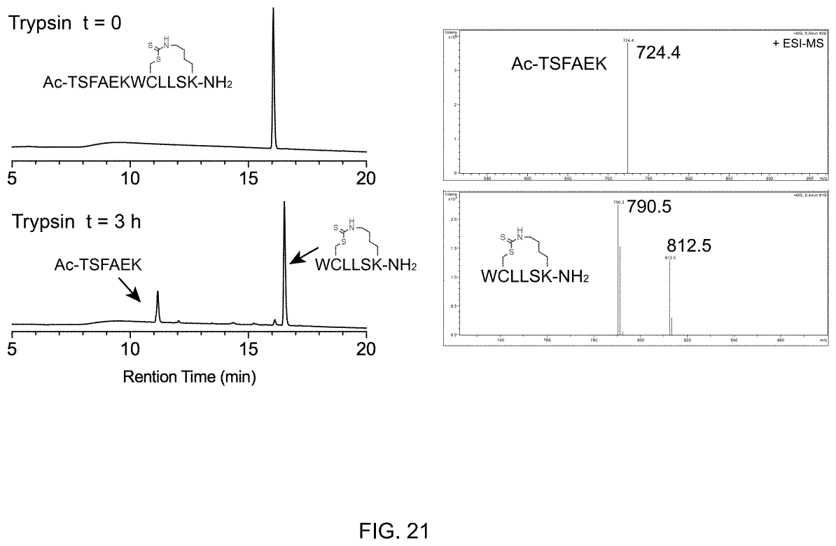

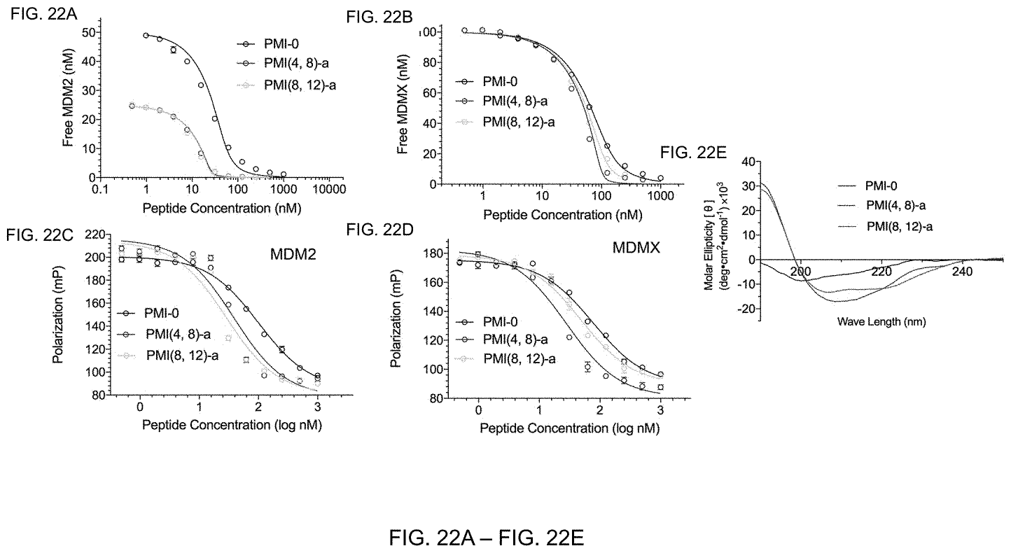

In the FP-based competitive binding assay, stapled peptide at increasing concentrations competed off a fluorescently tagged PMI peptide (10 nM) complexed with synthetic²⁵⁻¹⁰⁹ MDM2/²⁴⁻¹⁰⁸MDMX (50 nM), resulting in a progressive decrease in FP. The equilibrium inhibition constant, Ki, of stapled peptide for MDM2/MDMX was calculated as described. For SPR-based direct binding, different concentrations of stapled peptide were incubated with MDM2 at 50 nM or MDMX at 100 nM, unless indicated otherwise, and free MDM2/MDMX was quantified on a ¹⁵⁻²⁹p53-immobilized CM5 sensor chip to obtain the equilibrium dissociation constant, Kd, through non-linear regression analysis. Compared with the N-acetylated and C-amidated wild-type peptide PMI-0, PMI(4,8)-a and PMI(8,12)-a bound more strongly to MDM2 and MDMX. In fact, the crosslinked Lys-Cys pair at positions (4, 8) enhanced peptide binding to both proteins by one order of magnitude as measured (FIG. 22A-FIG. 22D). Both PMI(4,8)-a and PMI(8,12)-a partially adopted an α-helical structure in aqueous solution according to CD analyses (Table 7, FIG. 22E, FIG. 23). While not wishing to be bound by any particular theory, this results suggests that crosslinking Lys-Cys side chains stabilized peptide conformation productive for MDM2 and MDMX binding. Similarly, the stapled p53 peptide bound to MDM2 and MDMX roughly one order of magnitude stronger than ¹⁶⁻²⁷p53 (Table 7, FIG. 20A-FIG. 20E). Of note, the reversal of Lys-Cys (a) to Cys-Lys (b) in PMI was in general detrimental to peptide binding to MDM2 and MDMX (Table 7), indicating that the DTC crosslink is functionally unidirectional.

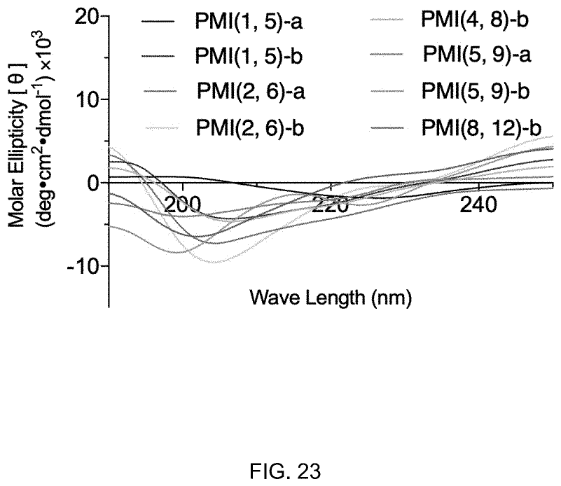

To structurally validate the DTC stapling chemistry, the co-crystal structures of MDM2-PMI(8,12)-a and MDMX-PMI(4,8)-a were solved at 1.8 and 2.7 Å resolution (Table 8), respectively, and compared with the structures of MDM2 and MDMX in complex with PMI (FIG. 24A and FIG. 24B).

TABLE 8
Data collection and refinement statistics

| Data collection | MDM2-PMI(8,12)-a | | MDMX-PMI(4,8)-a | |
|---|---|---|---|---|
| Wavelength, \acute{Å} | 0.97946 | | 0.97946 | |
| Space group | C222₁ | | P1 | |
| Cell parameters | | | | |
| a, b, c, Å | 90.8, 157.5, 196.7 | | 43.3, 47.7, 93.4 | |
| α, β, γ, ° | 90.0, 90.0, 90.0 | | 76.7, 89.9, 72.6 | |
| Complexes/a.u. | 12 | | 8 | |
| Resolution. (Å) | 50-1.8 | (1.83-1.8) | 50-2.7 | (2.75-2.7) |
| # of reflections | | | | |
| Total | 419,465 | | 31,027 | |
| Unique | 123,372 | | 16,330 | |
| R_{merg}^{b}, % | 7.6 | (29.9) | 13.2 | (61.4) |
| I/σ | 23.2 | (3.0) | 10.3 | (1.1) |
| Completeness, % | 95.5 | (97.9) | 86.0 | (78.7) |
| Redundancy | 3.4 | (3.4) | 1.9 | (1.8) |

Refinement Statistics

| | | |
|---|---|---|
| Resolution, Å | 41.7-1.80 | 50-2.7 |
| R^{c}, % | 19.8 | 28.1 |
| R_{free}^{d}, % | 24.6 | 33.5 |
| # of atoms | | |
| Protein | 9,547 | 5,910 |
| Water | 418 | 27 |
| Ligand/Ion | 182 | 115 |
| Overall B value (Å)² | | |
| Protein | 26.8 | 54.4 |
| Water | 24.8 | 38.9 |
| Ligand/Ion | 26.7 | 57.7 |
| Root mean square | | |
| deviation | | |
| Bond lengths, Å | 0.012 | 0.006 |
| Bond angles, ° | 1.79 | 1.14 |
| Ramachandran ^{e} | | |
| favored, % | 91.2 | 95.5 |
| allowed, % | 6.6 | 3.3 |
| outliers, % | 2.2 | 1.2 |
| PDB ID | 5VK0 | 5VK1 |

^{a}all data (outer shell).
^{b}R_{merge} = Σ|I − <I>|/ΣI, where I is the observed intensity and <I> is the average intensity obtained from multiple observations of symmetry-related reflections after rejections
^{c}R = Σ||Fₒ| − |F_{c}||/Σ|Fₒ|, where Fₒ and F_{c} are the observed and calculated structure factors, respectively
^{d}R_{free} = as defined by Brünger

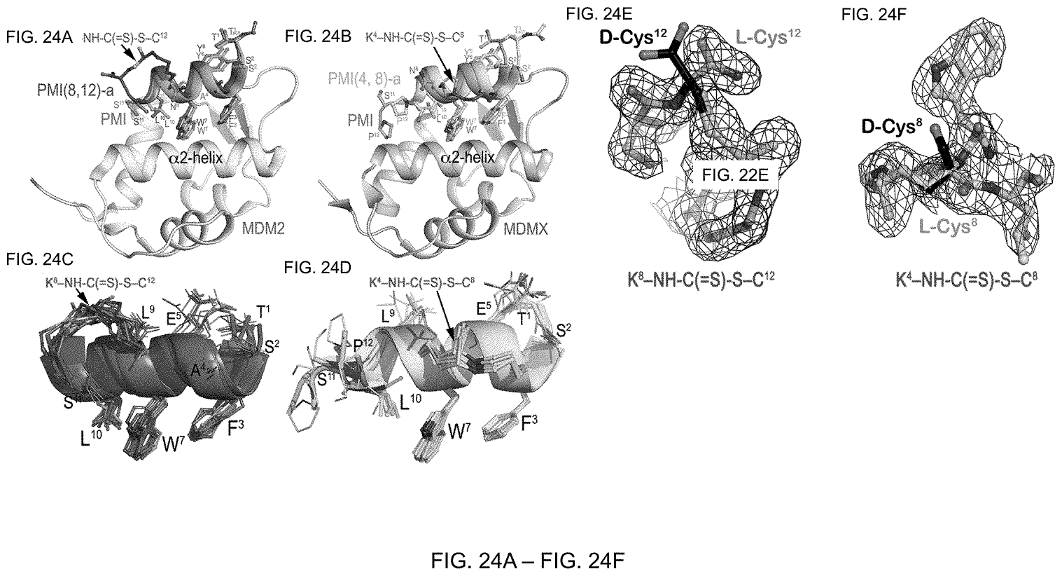

Both complexes crystallized with multiple copies in the asymmetric unit of the crystal—12 for MDM2-PMI(8,12)-a and 8 for MDMX-PMI(4,8)-a (Table 8, FIG. 25). Whereas all 12 residues could be built into each PMI(8,12)-a peptide complexed with MDM2, PMI(4,8)-a was fully defined in only 3 copies of the MDMX complex with no density observed for Ser11 and/or Pro12 (FIG. 24C and FIG. 24D). Alignment analysis of the PMI(8,12)-a conformation also indicated noticeable variability among the 12 copies of peptide, as evidenced by the root-mean-square deviation (RMSD) between the main-chain atoms in the range of 0.48-1.35 Å (Table 9). In both complexes, however, the crystallographic density for all atoms of the crosslink formed between Lys (i) and Cys (i+4) unambiguously defined the geometry of the DTC staple.

As shown in FIG. 24A, MDM2-bound PMI(8,12)-a largely overlapped with PMI, differing mainly in positions of the equivalent Ca atoms of residues Thr1-Trp7 with little change in the C-terminal region (Trp7-Ser11) (Table 9). More pronounced differences were observed between MDMX-bound

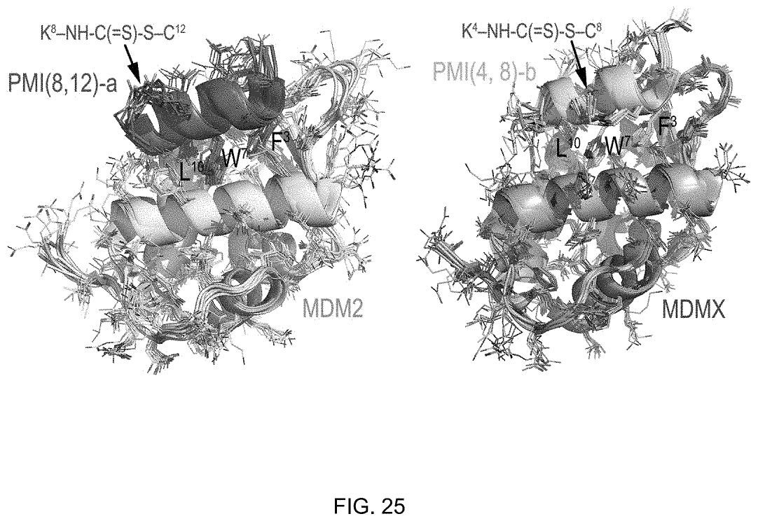

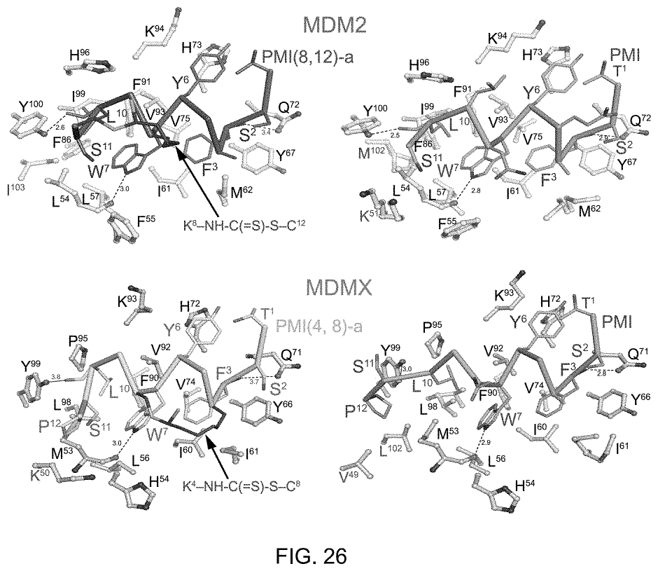

PMI(4,8)-a and PMI (Table 10), with the backbone of the former longitudinally shifting ˜2 Å toward one side of the p53-binding pocket of MDMX and closer to its α2-helix in relation to PMI (FIG. 24B). This shift, while increasing PMI(4,8)-a contacts with the edge of the cavity formed by the α2-helix of MDMX, reduced hydrophobic contacts and lengthened some hydrogen bonds seen in the PMI-MDMX complex (FIG. 26). The DTC staple rigidified, at positions (8,12), the C-terminus of PMI in a helical conformation and extended, at positions (4,8), the C-terminal helix of PMI from Leu9 to Ser11 (FIG. 24A and FIG. 24B). The rigidity of PMI(8,12)-a or PMI(4,8)-a increased to such an extent that the local buried surface area (BSA) slightly decreased as compared with the BSA contributed by PMI to its interface with MDM2/MDMX (FIG. 27). Although not wishing to be bound by any particular theory, this finding suggests that DTC stapling-enhanced binding may be energetically attributable to a reduced loss in entropy afforded by a pre-organized stable helix.

TABLE 9
The root mean square deviation (RMSD) between MDM2-PMI(8, 12)-a and MDM2-PMI complexes.

| | MDM2- | MDM2- | MDM2- | MDM2- | MDM2- | MDM2- | MDM2- | MDM2- |
|---|---|---|---|---|---|---|---|---|
| | PMI(8, 12)a | PMI(8, 12)b | PMI(8, 12)c | PMI(8, 12)d | PMI(8, 12)e | PMI(8, 12)f | PMI(8, 12)g | PMI(8, 12)h |
| MDM2- | — | 0.686 | 0.927 | 0.647 | 0.579 | 0.835 | 0.526 | 0.926 |
| PMI(8, 12)a | | | | | | | | |
| PMI(8, 12)a | — | 0.759 | 0.867 | 0.751 | 0.589 | 0.871 | 0.384 | 1.017 |
| MDM2-1 | — | 0.583 | 0.868 | 0.567 | 0.510 | 0.772 | 0.468 | 0.870 |
| MDM2- | 0.686 | — | 1.049 | 0.620 | 0.548 | 0.686 | 0.574 | 0.873 |
| PMI(8, 12)b | | | | | | | | |
| PMI(8, 12)b | 0.759 | — | 1.240 | 1.225 | 0.942 | 0.580 | 0.670 | 0.756 |
| MDM2b | 0.583 | — | 0.818 | 0.393 | 0.435 | 0.676 | 0.543 | 0.884 |
| MDM2- | 0.927 | 1.049 | — | 0.917 | 1.037 | 1.133 | 1.072 | 1.090 |
| PMI(8, 12)c | | | | | | | | |
| PMI(8, 12)c | 0.867 | 1.240 | — | 0.645 | 1.114 | 1.374 | 1.043 | 1.340 |
| MDM2c | 0.868 | 0.818 | — | 0.825 | 0.860 | 0.915 | 0.935 | 0.898 |
| MDM2- | 0.647 | 0.620 | 0.917 | — | 0.538 | 0.826 | 0.603 | 0.959 |
| PMI(8, 12)d | | | | | | | | |
| PMI(8, 12)d | 0.751 | 1.225 | 0.645 | — | 0.850 | 1.288 | 0.880 | 1.394 |
| MDM2d | 0.567 | 0.393 | 0.825 | — | 0.426 | 0.648 | 0.533 | 0.845 |
| MDM2- | 0.579 | 0.548 | 1.037 | 0.538 | — | 0.721 | 0.520 | 0.927 |
| PMI(8, 12)e | | | | | | | | |
| PMI(8, 12)e | 0.589 | 0.942 | 1.114 | 0.850 | — | 0.852 | 0.729 | 1.172 |
| MDM2e | 0.510 | 0.435 | 0.860 | 0.426 | — | 0.669 | 0.456 | 0.875 |
| MDM2- | 0.835 | 0.686 | 1.133 | 0.826 | 0.721 | — | 0.760 | 0.890 |
| PMI(8, 12)f | | | | | | | | |
| PMI(8, 12)f | 0.871 | 0.580 | 1.374 | 1.288 | 0.852 | — | 0.786 | 0.776 |
| MDM2f | 0.772 | 0.676 | 0.915 | 0.648 | 0.669 | — | 0.720 | 0.884 |
| MDM2- | 0.526 | 0.574 | 1.072 | 0.603 | 0.520 | 0.760 | — | 0.960 |
| PMI(8, 12)g | | | | | | | | |
| PMI(8, 12)g | 0.384 | 0.670 | 1.043 | 0.880 | 0.729 | 0.786 | — | 0.937 |
| MDM2g | 0.468 | 0.543 | 0.935 | 0.533 | 0.456 | 0.720 | — | 0.953 |
| MDM2- | 0.926 | 0.873 | 1.090 | 0.959 | 0.927 | 0.890 | 0.960 | — |
| PMI(8, 12)h | | | | | | | | |
| PMI(8, 12)h | 1.017 | 0.756 | 1.340 | 1.394 | 1.172 | 0.776 | 0.937 | — |
| MDM2h | 0.870 | 0.884 | 0.898 | 0.845 | 0.875 | 0.884 | 0.953 | — |
| MDM2- | 0.702 | 0.779 | 0.802 | 0.569 | 0.696 | 0.852 | 0.783 | 0.791 |
| PMI(8, 12)i | | | | | | | | |
| PMI(8, 12)i | 0.632 | 1.043 | 0.753 | 0.286 | 0.662 | 1.088 | 0.817 | 1.315 |
| MDM2i | 0.662 | 0.666 | 0.699 | 0.594 | 0.663 | 0.732 | 0.748 | 0.638 |
| MDM2- | 1.038 | 1.064 | 0.902 | 1.007 | 1.101 | 1.186 | 1.144 | 1.055 |
| PMI(8, 12)j | | | | | | | | |
| PMI(8, 12)j | 0.833 | 1.262 | 0.647 | 0.573 | 0.890 | 1.319 | 1.030 | 1.417 |
| MDM2j | 0.994 | 0.823 | 0.877 | 0.900 | 0.947 | 1.017 | 1.011 | 0.784 |
| MDM2- | 1.120 | 1.045 | 1.348 | 1.087 | 1.061 | 1.028 | 1.062 | 0.956 |
| PMI(8, 12)k | | | | | | | | |
| PMI(8, 12)k | 0.952 | 1.004 | 1.276 | 1.258 | 1.158 | 1.056 | 0.884 | 1.134 |
| MDM2k | 1.044 | 1.015 | 1.180 | 0.988 | 0.973 | 0.976 | 1.042 | 0.860 |
| MDM2- | 0.722 | 0.752 | 0.810 | 0.520 | 0.703 | 0.866 | 0.748 | 0.877 |
| PMI(8, 12)l | | | | | | | | |
| PMI(8, 12)l | 0.853 | 1.275 | 0.661 | 0.319 | 0.989 | 1.385 | 0.974 | 1.476 |
| MDM2I | 0.650 | 0.578 | 0.713 | 0.539 | 0.603 | 0.671 | 0.688 | 0.710 |
| MDM2- | 0.914 | 0.754 | 0.927 | 0.783 | 0.786 | 0.770 | 0.896 | 0.818 |
| PMI | | | | | | | | |
| PMI | 0.731 | 0.678 | 0.897 | 0.832 | 0.643 | 0.490 | 0.620 | 0.768 |
| MDM2 | 0.885 | 0.745 | 0.851 | 0.760 | 0.782 | 0.774 | 0.912 | 0.799 |

| | MDM2- | MDM2- | MDM2- | MDM2- | MDM2- |
|---|---|---|---|---|---|
| | PMI(8, 12)i | PMI(8, 12)j | PMI(8, 12)k | PMI(8, 12)l | PMI |
| MDM2- | 0.702 | 1.038 | 1.120 | 0.722 | 0.914 |
| PMI(8, 12)a | | | | | |
| PMI(8, 12)a | 0.632 | 0.833 | 0.952 | 0.853 | 0.731 |
| MDM2-1 | 0.662 | 0.994 | 1.044 | 0.650 | 0.885 |
| MDM2- | 0.779 | 1.064 | 1.045 | 0.752 | 0.754 |
| PMI(8, 12)b | | | | | |
| PMI(8, 12)b | 1.043 | 1.262 | 1.004 | 1.275 | 0.678 |
| MDM2b | 0.666 | 0.823 | 1.015 | 0.578 | 0.745 |
| MDM2- | 0.802 | 0.902 | 1.348 | 0.810 | 0.927 |
| PMI(8, 12)c | | | | | |
| PMI(8, 12)c | 0.753 | 0.647 | 1.276 | 0.661 | 0.897 |
| MDM2c | 0.699 | 0.877 | 1.180 | 0.713 | 0.851 |
| MDM2- | 0.569 | 1.007 | 1.087 | 0.520 | 0.783 |
| PMI(8, 12)d | | | | | |
| PMI(8, 12)d | 0.286 | 0.573 | 1.258 | 0.319 | 0.832 |
| MDM2d | 0.594 | 0.900 | 0.988 | 0.539 | 0.760 |
| MDM2- | 0.696 | 1.101 | 1.061 | 0.703 | 0.786 |
| PMI(8, 12)e | | | | | |
| PMI(8, 12)e | 0.662 | 0.890 | 1.158 | 0.989 | 0.643 |
| MDM2e | 0.663 | 0.947 | 0.973 | 0.603 | 0.782 |
| MDM2- | 0.852 | 1.186 | 1.028 | 0.866 | 0.770 |
| PMI(8, 12)f | | | | | |
| PMI(8, 12)f | 1.088 | 1.319 | 1.056 | 1.385 | 0.490 |
| MDM2f | 0.732 | 1.017 | 0.976 | 0.671 | 0.774 |
| MDM2- | 0.783 | 1.144 | 1.062 | 0.748 | 0.896 |
| PMI(8, 12)g | | | | | |
| PMI(8, 12)g | 0.817 | 1.030 | 0.884 | 0.974 | 0.620 |
| MDM2g | 0.748 | 1.011 | 1.042 | 0.688 | 0.912 |
| MDM2- | 0.791 | 1.055 | 0.956 | 0.877 | 0.818 |
| PMI(8, 12)h | | | | | |
| PMI(8, 12)h | 1.315 | 1.417 | 1.134 | 1.476 | 0.768 |
| MDM2h | 0.638 | 0.784 | 0.860 | 0.710 | 0.799 |
| MDM2- | — | 0.785 | 1.139 | 0.479 | 0.607 |
| PMI(8, 12)i | | | | | |
| PMI(8, 12)i | — | 0.501 | 1.337 | 0.314 | 0.560 |
| MDM2i | — | 0.600 | 1.013 | 0.494 | 0.594 |
| MDM2- | 0.785 | — | 1.299 | 0.850 | 0.878 |
| PMI(8, 12)j | | | | | |
| PMI(8, 12)j | 0.501 | — | 1.373 | 0.547 | 0.859 |
| MDM2j | 0.600 | — | 1.110 | 0.704 | 0.714 |
| MDM2- | 1.139 | 1.299 | — | 1.045 | 0.970 |
| PMI(8, 12)k | | | | | |
| PMI(8, 12)k | 1.337 | 1.373 | — | 1.340 | 0.829 |
| MDM2k | 1.013 | 1.110 | — | 0.923 | 0.984 |
| MDM2- | 0.479 | 0.850 | 1.045 | — | 0.606 |
| PMI(8, 12)l | | | | | |
| PMI(8, 12)l | 0.314 | 0.547 | 1.340 | — | 0.854 |
| MDM2I | 0.494 | 0.704 | 0.923 | — | 0.542 |
| MDM2- | 0.607 | 0.878 | 0.970 | 0.606 | — |
| PMI | | | | | |
| PMI | 0.560 | 0.859 | 0.829 | 0.854 | — |
| MDM2 | 0.594 | 0.714 | 0.984 | 0.542 | — |

Comparisons were made between 12 copies of MDM2-PMI(8, 12) complex (copies a, b, c, d, e, f, g, h, i, j, k, l) and one copy of MDM2-PMI complex.

TABLE 10
The root mean square deviation (RMSD) between MDMX-PMI(4, 8)-a and MDMX-PMI complexes.

| | MDMX- | MDMX- | MDMX- | MDMX- | MDMX- | MDMX- | MDMX- | MDMX- | MDMX- | MDMX- |
|---|---|---|---|---|---|---|---|---|---|---|
| | PMI(4, 8)a | PMI(4, 8)b | PMI(4, 8)c | PMI(4, 8)d | PMI(4, 8)e | PMI(4, 8)f | PMI(4, 8)g | PMI(4, 8)h | PMIa | PMIb |
| MDMX- | — | 0.976 | 0.600 | 0.677 | 0.936 | 0.762 | 0.586 | 0.589 | 1.254 | 1.270 |
| PMI(4, 8)a | | | | | | | | | | |
| PMI(4, 8)a | — | 2.188 | 1.011 | 1.091 | 1.815 | 1.100 | 0.721 | 0.274 | 1.059 | 1.081 |
| MDMXa | — | 0.464 | 0.479 | 0.538 | 0.461 | 0.641 | 0.529 | 0.594 | 0.966 | 0.969 |
| MDMX- | 0.976 | — | 0.618 | 0.545 | 0.660 | 0.830 | 0.724 | 0.611 | 1.534 | 1.574 |
| PMI(4, 8)b | | | | | | | | | | |
| PMI(4, 8)b | 2.188 | — | 1.018 | 0.752 | 1.089 | 1.002 | 1.411 | 0.590 | 2.461 | 2.582 |
| MDMXb | 0.464 | — | 0.505 | 0.454 | 0.504 | 0.735 | 0.526 | 0.583 | 0.853 | 0.855 |
| MDMX- | 0.600 | 0.618 | — | 0.684 | 0.498 | 0.761 | 0.599 | 0.672 | 1.096 | 1.096 |
| PMI(4, 8)c | | | | | | | | | | |
| PMI(4, 8)c | 1.011 | 1.018 | — | 1.029 | 0.946 | 1.010 | 1.003 | 1.060 | 0.902 | 0.902 |
| MDMXc | 0.479 | 0.505 | — | 0.575 | 0.368 | 0.683 | 0.491 | 0.575 | 0.939 | 0.940 |
| MDMX- | 0.677 | 0.545 | 0.684 | — | 0.543 | 0.756 | 0.647 | 0.627 | 1.106 | 1.107 |
| PMI(4, 8)d | | | | | | | | | | |
| PMI(4, 8)d | 1.091 | 0.752 | 1.029 | — | 0.732 | 0.464 | 1.157 | 0.354 | 1.318 | 1.319 |
| MDMXd | 0.538 | 0.454 | 0.575 | — | 0.490 | 0.744 | 0.471 | 0.632 | 0.845 | 0.847 |
| MDMX- | 0.936 | 0.660 | 0.498 | 0.543 | — | 0.733 | 0.711 | 0.564 | 1.508 | 1.541 |
| PMI(4, 8)e | | | | | | | | | | |
| PMI(4, 8)e | 1.815 | 1.089 | 0.946 | 0.732 | — | 1.036 | 1.338 | 0.585 | 2.112 | 2.198 |
| MDMXe | 0.461 | 0.504 | 0.368 | 0.490 | — | 0.617 | 0.463 | 0.552 | 0.896 | 0.899 |
| MDMX- | 0.762 | 0.830 | 0.761 | 0.756 | 0.733 | — | 0.816 | 0.746 | 1.388 | 1.389 |
| PMI(4, 8)f | | | | | | | | | | |
| PMI(4, 8)f | 1.100 | 1.002 | 1.010 | 0.464 | 1.036 | — | 0.945 | 0.319 | 1.104 | 1.105 |
| MDMXf | 0.641 | 0.735 | 0.683 | 0.744 | 0.617 | — | 0.710 | 0.774 | 1.113 | 1.114 |
| MDMX- | 0.586 | 0.724 | 0.599 | 0.647 | 0.711 | 0.816 | — | 0.501 | 1.036 | 1.037 |
| PMI(4, 8)g | | | | | | | | | | |
| PMI(4, 8)g | 0.721 | 1.411 | 1.003 | 1.157 | 1.338 | 0.945 | — | 0.366 | 0.453 | 0.456 |
| MDMXg | 0.529 | 0.526 | 0.491 | 0.471 | 0.463 | 0.710 | — | 0.496 | 0.914 | 0.916 |
| MDMX- | 0.589 | 0.611 | 0.672 | 0.627 | 0.564 | 0.746 | 0.501 | — | 1.066 | 1.067 |
| PMI(4, 8)h | | | | | | | | | | |
| PMI(4, 8)h | 0.274 | 0.590 | 1.060 | 0.354 | 0.585 | 0.319 | 0.366 | — | 0.362 | 0.364 |
| MDMXh | 0.594 | 0.583 | 0.575 | 0.632 | 0.552 | 0.774 | 0.496 | — | 0.955 | 0.956 |
| MDMX- | 1.254 | 1.534 | 1.096 | 1.106 | 1.508 | 1.388 | 1.036 | 1.066 | — | |
| PMIa | | | | | | | | | | |
| PMIa | 1.059 | 2.461 | 0.902 | 1.318 | 2.112 | 1.104 | 0.453 | 0.362 | — | 0.107 |
| MDMXa | 0.966 | 0.853 | 0.939 | 0.845 | 0.896 | 1.113 | 0.914 | 0.955 | — | 0.289 |
| | | | | | | | | | | 0.017 |
| MDMX- | 1.270 | 1.574 | 1.096 | 1.107 | 1.541 | 1.389 | 1.037 | 1.067 | 0.107 | — |
| PMIb | | | | | | | | | | |
| PMIb | 1.081 | 2.582 | 0.902 | 1.319 | 2.198 | 1.105 | 0.456 | 0.364 | 0.289 | — |
| MDMXb | 0.969 | 0.855 | 0.940 | 0.847 | 0.899 | 1.114 | 0.916 | 0.956 | 0.017 | — |

Comparisons were made between 8 copies of MDMX-PMI(4, 8) complex (copies a, b, c, d, e, f, g, h) and two copies of MDM-PMI complex (copies a, b).

The DTC structure of the predominant epimer was deduced from the crystal structures of PMI(4,8)-a and PMI(8,12)-a in respective complex with MDMX and MDM2, where Cys8 or Cys12 remained as an L-amino acid residue as shown in the electron density maps (FIG. 24E and FIG. 24F). The biochemical and biophysical findings on the DTC-stapled peptides unambiguously demonstrated their purity and stereo-selectivity for L-Cys, though.

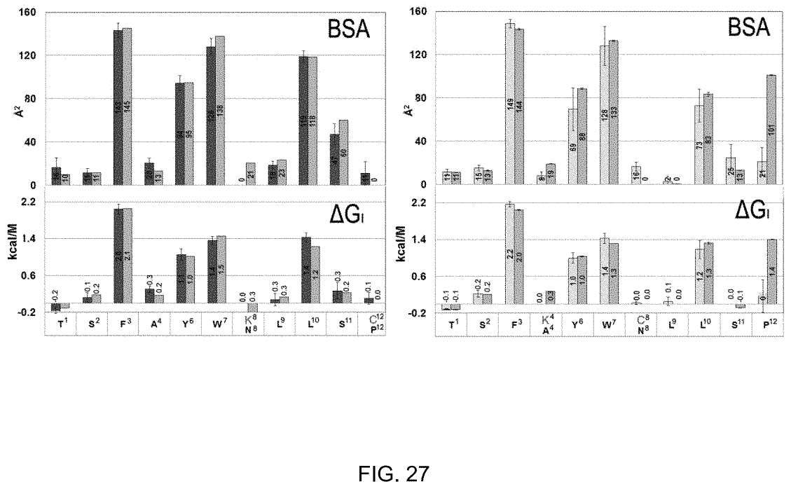

Side chain stapled peptides are structurally rigidified as compared with their linear counterparts and, thus, expected to be more resistant to proteolysis in vivo. HPLC and ESI-MS were used to evaluate the proteolytic stability of PMI(8,12)-a versus PMI-0 at 100 μM in cell culture medium in the presence of 25 μg/mL cathepsin G—an intracellular protease with dual specificities for both basic and bulky hydrophobic residues. As shown in FIG. 28A, while PMI-0 was fully degraded by the enzyme within 30 min of co-incubation at room temperature, the DTC-stapled peptide was substantially more stable with a half-life of ˜8 h under identical conditions. Similar results were obtained using human serum (FIG. 28B). Of note, the DTC structure is also stable in the presence of reduced glutathione (GST). When PMI(8,12)-a was incubated at 25° C. in PBS buffer with GST at 10 mM—a physiological concentration, no apparent breakdown of the DTC structure was observed over 24 h (FIG. 28A-FIG. 28B).

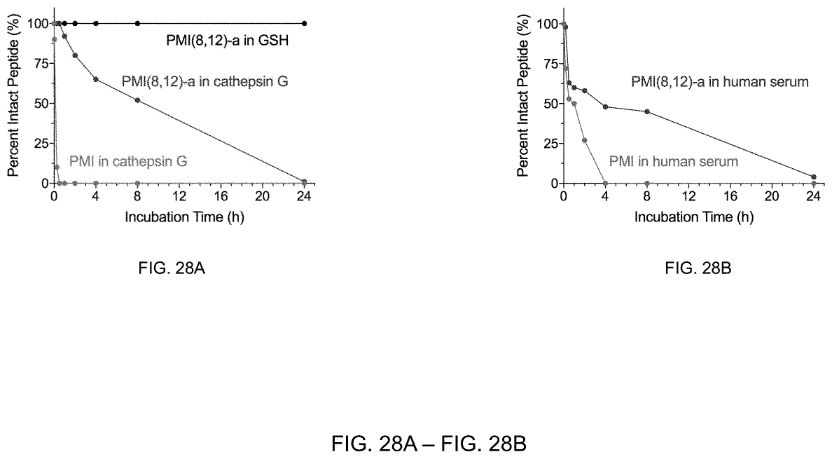

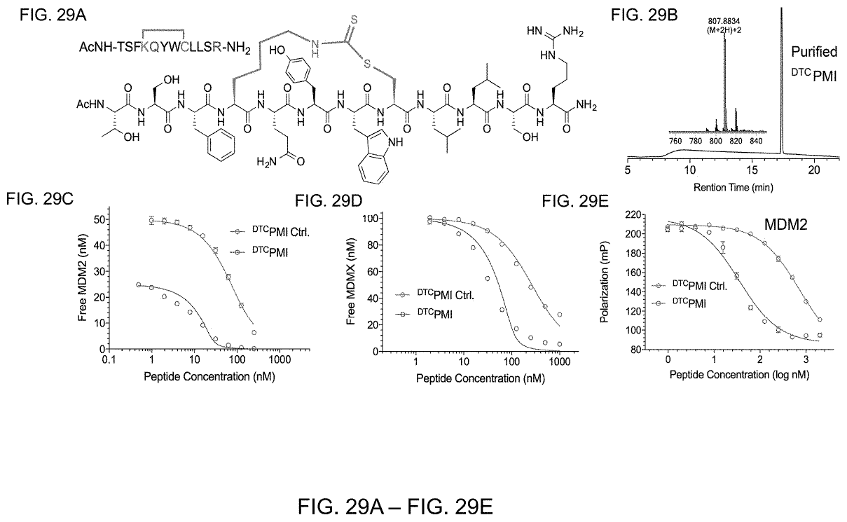

Verdine and colleagues have shown that structurally permissible stapling of a p53 peptide, while enhancing α-helicity and improving MDM2 binding, is not sufficient to endow the peptide with an ability to kill tumor cells. Although cationicity is not a universal molecular signature of cell-penetrating peptides, it plays a critical role in the ability of stapled peptides to traverse the cell membrane to exert biological activity. The DTC-stapled peptides described herein carrying a net charge of either 0 or −1 showed little cytotoxicity against HCT116p53^{+/+} and HCT116p53^{-/-} cells at up to 100 μM (FIG. 11). Using PMI(4,8)-a as a template, two cationicity-enhancing mutations were made, E5Q and P12R, resulting in a DTC-stapled peptide termed ^{DTC}PMI with a +1 net charge (FIG. 29A-FIG. 29B). Confocal microscopic analysis of HCT116 cells treated with 20 μM^{DTC}PMI N-terminally conjugated to fluorescein (FITC) revealed a diffused intracellular localization of the peptide (FIG. 30), confirming the ability of ^{DTC}PMI to permeabilize the cell membrane.

Compared with its unstapled control peptide, Ac-TSFKQYWCLLSR-NH₂, DTC crosslinking increased peptide binding affinity for MDM2 and MDMX by 50-fold as measured by SPR (FIG. 29C-FIG. 29D, Table 6) or ˜20-fold by FP (FIG. 29E-FIG. 29F, Table 6), making ^{DTC}PMI (K_{d}=0.87 and 3.9 nM for MDM2 and MDMX, respectively) a strong dual-specificity peptide antagonist against both proteins. Of note, ^{DTC}PMI also displayed a strong tendency to adopt α-helix on its own in aqueous solution (Table 6, FIG. 29G), likely contributing energetically to its high-affinity binding to both MDM2 and MDMX. As is the case with ^{DTC}PMI, PMI(4,8)-a and PMI(8,12)-a, while stapling-enhanced α-helicity qualitatively predicts strong peptide binding to MDM2/MDMX, a quantitative correlation appears lacking, due, in part, to the deficiency of CD spectroscopy in accurate measurements of α-helicity of small peptides that are generally disordered and conformationally heterogeneous.

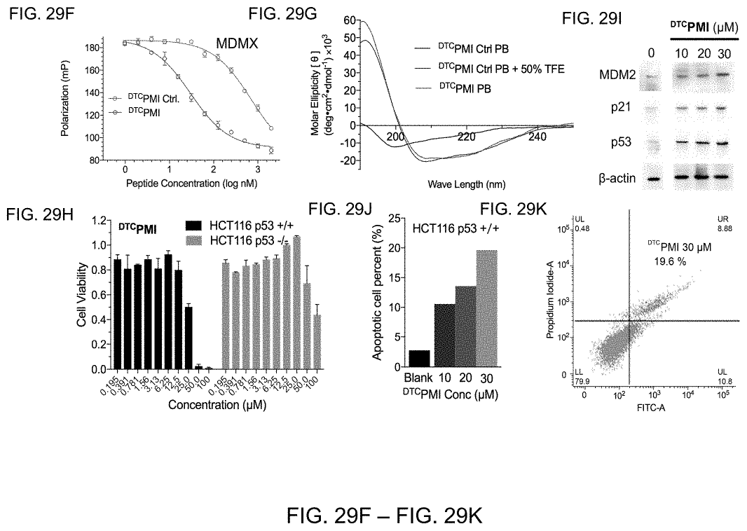

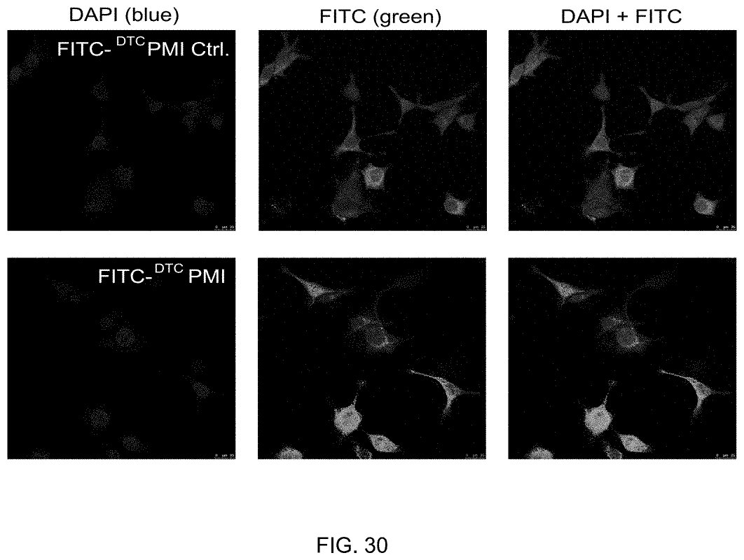

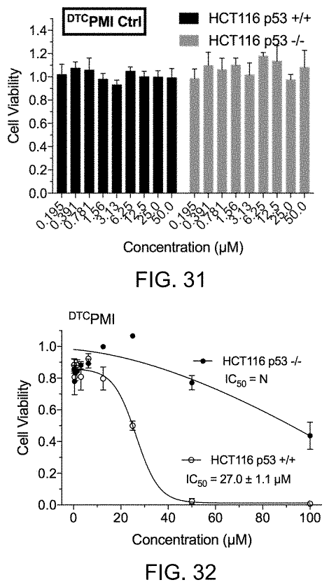

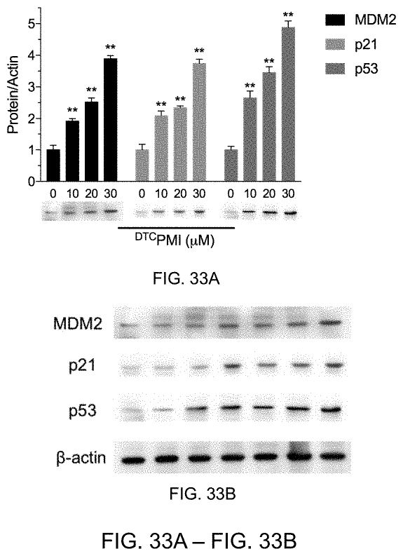

To functionally validate ^{DTC}PMI, it and its unstapled control were subjected to a cell viability assay using HCT116 p5.3^{+/+} and p5.3^{-/-} cells. Lane and colleagues previously reported that serum proteins were inhibitory against the tumor-killing activity of hydrocarbon-stapled peptide antagonists of MDM2. To mitigate the potential effect of serum binding on peptide activity, cells were treated in serum-free media for 8 h, followed by addition of serum supplements and incubation for 64 h. While the control peptide exhibited no anti-proliferative activity against both cell lines at concentrations of up to 50 μM (FIG. 31), ^{DTC}PMI displayed p53-dependent growth inhibitory activity against HCT116 p53^{+/+}, but not HCT116 p53^{+/+}, with an IC₅₀ value of ˜25 μM at 72 h (FIG. 29H, FIG. 32). To investigate the mechanisms of killing of HCT116 p53^{+/+} by ^{DTC}PMI, the expression of MDM2, p53 and p21 was analysed by Western blotting. As shown in FIG. 29I and FIG. 33A-FIG. 33B, 8 h after treatment with ^{DTC}PMI, dose-dependent induction of p53, MDM2 and p21 became evident in HCT116 p53^{+/+} cells. Consistent with this result, dose-dependent induction of apoptosis of HCT116 p53^{+/+} cells by ^{DTC}PMI was verified by fluorescence-activated cell sorting (FACS) (FIG. 29J, FIG. 29K, FIG. 15). By contrast, no obvious apoptosis of HCT116 p53^{-/-} cells was observed by FACS under identical treatment conditions (FIG. 34). Taken together, these findings support that ^{DTC}PMI actively traversed the cell membrane and killed tumor cells by antagonizing MDM2 to reactivate the p53 pathway.

Of note, at the high concentration of 100 μM, ^{DTC}PMI significantly reduced cell viability of HCT116 p53^{-/-} cells as well (FIG. 29H). Although not wishing to be bound by any particular theory, this result may be due to the fact that the MDM2 antagonist Nutlin-3 also kills HCT116 p53^{-/-} at high concentrations, in part by disrupting MDM2 interactions with p73, a member of the p53 family that transcriptionally induces cell-cycle arrest and/or apoptosis. In fact, recent data demonstrate that p73 is elevated to compensate for p53 loss when MDM2 is deleted in p53-null tumor cells. It is therefore plausible that the observed killing of HCT116 p53^{-/-} by ^{DTC}PMI at high concentrations arises from its p53-independent on-target activity, potentially extending ^{DTC}PMI to the treatment of p53-deficient cancers as well.

Aside from the simplicity of using natural amino acids, the DTC chemistry may offer an added advantage over the hydrocarbon stapling technique: peptide solubility. If stapling severely decreases peptide solubility, it can potentially limit drug concentration in vivo, thus therapeutic efficacy. For direct comparison, Ac-TSFXQYWXLLSR-NH₂ was stapled with a hydrocarbon linkage between X residues at positions 4 and 8 (X═(S)-2-(4′-pentenyl)alanine), yielding a hydrocarbon stapled peptide termed ^{HC}PMI that differs only in the crosslink from ^{DTC}PMI. ^{DTC}PMI and ^{HC}PMI were each suspended at 20 mg/ml in PBS, followed by a 2-fold serial dilution and OD measurements at 600 nm. As shown in FIG. 35, while ^{DTC}PMI was soluble at a concentration of >10 mg/ml, the solubility of ^{HC}PMI was significantly lower, at ˜0.3 mg/ml. Since dithiocarbamate contains multiple hydrogen bond donors/acceptors, the DTC staple is expected to be more soluble than all-hydrocarbon crosslinks.

### CONCLUSIONS

This Example demonstrates a novel stapling strategy for peptide drug design by taking advantage of the DTC chemistry to crosslink the side chains of the two natural amino acid residues Lys and Cys at (i, i+4) positions. The DTC staple, structurally validated, induced the formation of and stabilized a productive α-helical conformation of PMI—a dual-specificity peptide antagonist of MDM2 and MDMX, enabling it to traverse the cell membrane and kill tumor cells by reactivating the p53 pathway. DTC stapling functionally rescued PMI that, on its own, failed to activate p53 in vitro and in vivo due to its poor membrane permeability and susceptibility to proteolytic degradation. DTC stapling offers a better peptide aqueous solubility over hydrocarbon stapling. Compared with other known stapling techniques, the solution-based DTC chemistry is simple, cost-effective, regio-specific, and environmentally friendly, promising an important new tool for peptide drug discovery and development for a variety of human diseases.

### REFERENCES

1. (a) Azzarito, V.; Long, K.; Murphy, N. S.; Wilson, A. J., Nat Chem 2013, 5 (3), 161; (b) Keskin, O.; Gursoy, A.; Ma, B.; Nussinov, R., Chem Rev 2008, 108 (4), 1225.
2. (a) Zhao, L.; Lu, W., Curr Opin Chem Biol 2014, 22, 56; (b) Liu, M.; Li, C.; Pazgier, M.; Li, C.; Mao, Y.; Lv, Y.; Gu, B.; Wei, G.; Yuan, W.; Zhan, C.; Lu, W. Y.; Lu, W., Proc Natl Acad Sci USA 2010, 107 (32), 14321; (c) Liu, M.; Pazgier, M.; Li, C.; Yuan, W.; Li, C.; Lu, W., Angew Chem Int Ed Engl 2010, 49 (21), 3649.
3. (a) Lau, Y. H.; de Andrade, P.; Wu, Y.; Spring, D. R., Chem Soc Rev 2015, 44 (1), 91; (b) Kutchukian, P. S.; Yang, J. S.; Verdine, G. L.; Shakhnovich, E. I., J Am Chem Soc 2009, 131 (13), 4622; (c) Lau, Y. H.; Wu, Y.; Rossmann, M.; Tan, B. X.; de Andrade, P.; Tan, Y. S.; Verma, C.; McKenzie, G. J.; Venkitaraman, A. R.; Hyvonen, M.; Spring, D. R., Angew Chem Int Ed Engl 2015, 54 (51), 15410; (d) Lautrette, G.; Touti, F.; Lee, H. G.; Dai, P.; Pentelute, B. L., J Am Chem Soc 2016, 138 (27), 8340; (e) Peraro, L.; Zou, Z.; Makwana, K. M.; Cummings, A. E.; Ball, H. L.; Yu, H.; Lin, Y. S.; Levine, B.; Kritzer, J. A., J Am Chem Soc 2017, 139 (23), 7792; (f) Speltz, T. E.; Fanning, S. W.; Mayne, C. G.; Fowler, C.; Tajkhorshid, E.; Greene, G. L.; Moore, T. W., Angew Chem Int Ed Engl 2016, 55 (13), 4252; (g) Wiedmann, M. M.; Tan, Y. S.; Wu, Y.; Aibara, S.; Xu, W.; Sore, H. F.; Verma, C. S.; Itzhaki, L.; Stewart, M.; Brenton, J. D.; Spring, D. R., Angew Chem Int Ed Engl 2017, 56 (2), 524.
4. (a) Bernal, F.; Tyler, A. F.; Korsmeyer, S. J.; Walensky, L. D.; Verdine, G. L., J Am Chem Soc 2007, 129 (9), 2456; (b) Schafmeister, C. E.; Po, J.; Verdine, G. L., J Am Chem Soc 2000, 122, 5891; (c) Verdine, G. L.; Hilinski, G. J., Methods Enzymol 2012, 503, 3.
5. (a) Baek, S.; Kutchukian, P. S.; Verdine, G. L.; Huber, R.; Holak, T. A.; Lee, K. W.; Popowicz, G. M., J Am Chem Soc 2012, 134 (1), 103; (b) Bernal, F.; Wade, M.; Godes, M.; Davis, T. N.; Whitehead, D. G.; Kung, A. L.; Wahl, G. M.; Walensky, L. D., Cancer Cell 2011, 18 (5), 411; (c) Brown, C. J.; Quah, S. T.; Jong, J.; Goh, A. M.; Chiam, P. C.; Khoo, K. H.; Choong, M. L.; Lee, M. A.; Yurlova, L.; Zolghadr, K.; Joseph, T. L.; Verma, C. S.; Lane, D. P., ACS Chem Biol 2013, 8 (3), 506; (d) Chang, Y. S.; Graves, B.; Guerlavais, V.; Tovar, C.; Packman, K.; To, K. H.; Olson, K. A.; Kesavan, K.; Gangurde, P.; Mukherjee, A.; Baker, T.; Darlak, K.; Elkin, C.; Filipovic, Z.; Qureshi, F. Z.; Cai, H.; Berry, P.; Feyfant, E.; Shi, X. E.; Horstick, J.; Annis, D. A.; Manning, A. M.; Fotouhi, N.; Nash, H.; Vassilev, L. T.; Sawyer, T. K., Proc Natl Acad Sci USA 2013, 110 (36), E3445; (e) LaBelle, J. L.; Katz, S. G.; Bird, G. H.; Gavathiotis, E.; Stewart, M. L.; Lawrence, C.; Fisher, J. K.; Godes, M.; Pitter, K.; Kung, A. L.; Walensky, L. D., J Clin Invest 2012, 122 (6), 2018; (f) Moellering, R. E.; Cornejo, M.; Davis, T. N.; Del Bianco, C.; Aster, J. C.; Blacklow, S. C.; Kung, A. L.; Gilliland, D. G.; Verdine, G. L.; Bradner, J. E., Nature 2009, 462 (7270), 182; (g) Phillips, C.; Roberts, L. R.; Schade, M.; Bazin, R.; Bent, A.; Davies, N. L.; Moore, R.; Pannifer, A. D.; Pickford, A. R.; Prior, S. H.; Read, C. M.; Scott, A.; Brown, D. G.; Xu, B.; Irving, S. L., J Am Chem Soc 2011, 133 (25), 9696; (h) Takada, K.; Zhu, D.; Bird, G. H.; Sukhdeo, K.; Zhao, J. J.; Mani, M.; Lemieux, M.; Carrasco, D. E.; Ryan, J.; Horst, D.; Fulciniti, M.; Munshi, N. C.; Xu, W.; Kung, A. L.; Shivdasani, R. A.; Walensky, L. D.; Carrasco, D. R., Sci Transl Med 2012, 4 (148), 148ra117; (i) Walensky, L. D.; Kung, A. L.; Escher, I.; Malia, T. J.; Barbuto, S.; Wright, R. D.; Wagner, G.; Verdine, G. L.; Korsmeyer, S. J., Science 2004, 305 (5689), 1466; (j) Grossmann, T. N.; Yeh, J. T.; Bowman, B. R.; Chu, Q.; Moellering, R. E.; Verdine, G. L., Proc Natl Acad Sci USA 2012, 109 (44), 17942; (k) Chen, X.; Tai, L.; Gao, J.; Qian, J.; Zhang, M.; Li, B.; Xie, C.; Lu, L.; Lu, W.; Lu, W., J Control Release 2015, 218, 29.
6. (a) Wade, M.; Li, Y. C.; Wahl, G. M., Nat Rev Cancer 2013, 13 (2), 83; (b) Khoo, K. H.; Verma, C. S.; Lane, D. P., Nat Rev Drug Discov 2014, 13 (3), 217; (c) Zhang, Q.; Zeng, S. X.; Lu, H., Subcell Biochem 2014, 85, 281.
7. Meric-Bernstam, F.; Saleh, M. N.; Infante, J. R.; Goel, S.; Falchook, G. S.; Shapiro, G.; Chung, K. Y.; Conry, R. M.; Hong, D. S.; Wang, J. S.-Z.; Steidl, U.; Walensky, L. D.; Guerlavais, V.; Payton, M.; Annis, D. A.; Aivado, M.; Patel, M. R., Journal of Clinical Oncology 2017, 35 (15_suppl), 2505.
8. (a) Chalker, J. M.; Lercher, L.; Rose, N. R.; Schofield, C. J.; Davis, B. G., Angew Chem Int Ed Engl 2012, 51 (8), 1835; (b) Haj-Yahya, N.; Hemantha, H. P.; Meledin, R.; Bondalapati, S.; Seenaiah, M.; Brik, A., Org Lett 2014, 16 (2), 540.
9. (a) Azizi, N.; Aryanasab, F.; Saidi, M. R., Org Lett 2006, 8 (23), 5275; (b) Azizi, N.; Aryanasab, F.; Torkiyan, L.; Ziyaei, A.; Saidi, M. R., J Org Chem 2006, 71 (9), 3634.
10. (a) Li, C.; Pazgier, M.; Li, C.; Yuan, W.; Liu, M.; Wei, G.; Lu, W. Y.; Lu, W., J Mol Biol 2010, 398 (2), 200; (b) Pazgier, M.; Liu, M.; Zou, G.; Yuan, W.; Li, C.; Li, C.; Li, J.; Monbo, J.; Zella, D.; Tarasov, S. G.; Lu, W., Proc Natl Acad Sci USA 2009, 106 (12), 4665.
11. Kent, S. B., Chem Soc Rev 2009, 38 (2), 338.
12. (a) Zhan, C.; Varney, K.; Yuan, W.; Zhao, L.; Lu, W., J Am Chem Soc 2012, 134 (15), 6855; (b) Zhan, C.; Zhao, L.; Wei, X.; Wu, X.; Chen, X.; Yuan, W.; Lu, W. Y.; Pazgier, M.; Lu, W., J Med Chem 2012, 55 (13), 6237; (c) Chen, X.; Gohain, N.; Zhan, C.; Lu, W. Y.; Pazgier, M.; Lu, W., Oncogene 2016, 35 (15), 1919.
13. Polanowska, J.; Krokoszynska, I.; Czapinska, H.; Watorek, W.; Dadlez, M.; Otlewski, J., Biochim Biophys Acta 1998, 1386 (1), 189.
14. Deponte, M., Biochim Biophys Acta 2013, 1830 (5), 3217.
15. Hu, B.; Gilkes, D. M.; Chen, J., Cancer Res 2007, 67 (18), 8810. 1.
16 (a) Dawson, P. E.; Kent, S. B., Annu Rev Biochem 2000, 69, 923; (b) Dawson, P. E.; Muir, T. W.; Clark-Lewis, I.; Kent, S. B., Science 1994, 266 (5186), 776; (c) Pazgier, M.; Liu, M.; Zou, G.; Yuan, W.; Li, C.; Li, C.; Li, J.; Monbo, J.; Zella, D.; Tarasov, S. G.; Lu, W., Proc Natl Acad Sci USA 2009, 106 (12), 4665.
17. Ding, K.; Lu, Y.; Nikolovska-Coleska, Z.; Qiu, S.; Ding, Y.; Gao, W.; Stuckey, J.; Krajewski, K.; Roller, P. P.; Tomita, Y.; Parrish, D. A.; Deschamps, J. R.; Wang, S., J Am Chem Soc 2005, 127 (29), 10130.
18. Wang, D.; Chen, K.; Kulp Iii, J. L.; Arora, P. S., J Am Chem Soc 2006, 128 (28), 9248.
19. Otwinowski, Z.; Minor, W.; Charles W. Carter, Jr., Processing of X-ray diffraction data collected in oscillation mode. In Methods in Enzymology, Academic Press: 1997; Vol. Volume 276, pp 307.
20. Murshudov, G. N.; Vagin, A. A.; Dodson, E. J., Acta Crystallogr D Biol Crystallogr 1997, 53 (Pt 3), 240.
21. Emsley, P.; Cowtan, K., Acta Crystallogr D Biol Crystallogr 2004, 60 (Pt 12 Pt 1), 2126. L. Malavolta, M. R. Pinto, J. H. Cuvero, C. R. Nakaie, Protein Sci., 2006, 15, 1476-1488.
22. Y. H. Lau, P. de Andrade, Y. Wu, D. R. Spring, Chem. Soc. Rev., 2015, 44, 91-102.
23 P. S. Kutchukian, J. S. Yang, G. L. Verdine, E. I. Shakhnovich, J. Am. Chem. Soc., 2009, 131, 4622-4627.
24 Y. H. Lau, Y. Wu, M. Rossmann, B. X. Tan, P. de Andrade, Y. S. Tan, C. Verma, G. J. McKenzie, A. R. Venkitaraman, M. Hyvonen, D. R. Spring, Angew. Chem. Int. Ed. Engl., 2015, 54, 15410-15413.
25 G. Lautrette, F. Touti, H. G. Lee, P. Dai, B. L. Pentelute, J. Am. Chem. Soc., 2016, 138, 8340-8343.
26 L. Peraro, Z. Zou, K. M. Makwana, A. E. Cummings, H. L. Ball, H. Yu, Y. S. Lin, B. Levine, J. A. Kritzer, J. Am. Chem. Soc., 2017, 139, 7792-7802.
27 T. E. Speltz, S. W. Fanning, C. G. Mayne, C. Fowler, E. Tajkhorshid, G. L. Greene, T. W. Moore, Angew. Chem. Int. Ed. Engl., 2016, 55, 4252-4255.
28 M. M. Wiedmann, Y. S. Tan, Y. Wu, S. Aibara, W. Xu, H. F. Sore, C. S. Verma, L. Itzhaki, M. Stewart, J. D. Brenton, D. R. Spring, Angew. Chem. Int. Ed. Engl., 2017, 56, 524-529.
29 Y. Wu, Y. H. Li, X. Li, Y. Zou, H. L. Liao, L. Liu, Y. G. Chen, D. Bierer, H. G. Hu, Chem. Sci., 2017, 8, 7368-7373.
30 C. M. Fadzen, J. M. Wolfe, C. F. Cho, E. A. Chiocca, S. E. Lawler, B. L. Pentelute, J. Am. Chem. Soc., 2017, 139, 15628-15631.
31 C. M. Grison, G. M. Burslem, J. A. Miles, L. K. A. Pilsl, D. J. Yeo, Z. Imani, S. L. Warriner, M. E. Webb, A. J. Wilson, Chem. Sci., 2017, 8, 5166-5171.
32 G. T. Perell, R. L. Staebell, M. Hairani, A. Cembran, W. C. K. Pomerantz, ChemBioChem, 2017, 18, 1836-1844.
33 H. K. Cui, Y. Guo, Y. He, F. L. Wang, H. N. Chang, Y. J. Wang, F. M. Wu, C. L. Tian, L. Liu, Angew. Chem. Int. Ed. Engl., 2013, 52, 9558-9562.
34 Y. Guo, D. M. Sun, F. L. Wang, Y. He, L. Liu, C. L. Tian, Angew. Chem. Int. Ed. Engl., 2015, 54, 14276-14281.
35 X. Li, Y. Zou, H. G. Hu, Chinese Chem. Lett., 2018, 29, 1088-1092.
36 F. Bernal, A. F. Tyler, S. J. Korsmeyer, L. D. Walensky, G. L. Verdine, J. Am. Chem. Soc., 2007, 129, 2456-2457.
37 C. E. Schafmeister, J. Po, G. L. Verdine, J. Am. Chem. Soc., 2000, 122, 5891-5892.
38 G. L. Verdine, G. J. Hilinski, Methods Enzymol., 2012, 503, 3-33.
39 S. Baek, P. S. Kutchukian, G. L. Verdine, R. Huber, T. A. Holak, K. W. Lee, G. M. Popowicz, J. Am. Chem. Soc., 2012, 134, 103-106.
40 F. Bernal, M. Wade, M. Godes, T. N. Davis, D. G. Whitehead, A. L. Kung, G. M. Wahl, L. D. Walensky, Cancer Cell, 2011, 18, 411-422.
41 C. J. Brown, S. T. Quah, J. Jong, A. M. Goh, P. C. Chiam, K. H. Khoo, M. L. Choong, M. A. Lee, L. Yurlova, K. Zolghadr, T. L. Joseph, C. S. Verma, D. P. Lane, ACS Chem. Biol., 2013, 8, 506-512.
42 Y. S. Chang, B. Graves, V. Guerlavais, C. Tovar, K. Packman, K. H. To, K. A. Olson, K. Kesavan, P. Gangurde, A. Mukherjee, T. Baker, K. Darlak, C. Elkin, Z. Filipovic, F. Z. Qureshi, H. Cai, P. Berry, E. Feyfant, X. E. Shi, J. Horstick, D. A. Annis, A. M. Manning, N. Fotouhi, H. Nash, L. T. Vassilev, T. K. Sawyer, Proc. Natl. Acad. Sci. U.S.A., 2013, 110, E3445-3454.
43 J. L. LaBelle, S. G. Katz, G. H. Bird, E. Gavathiotis, M. L. Stewart, C. Lawrence, J. K. Fisher, M. Godes, K. Pitter, A. L. Kung, L. D. Walensky, J. Clin. Invest., 2012, 122, 2018-2031.
44 R. E. Moellering, M. Cornejo, T. N. Davis, C. Del Bianco, J. C. Aster, S. C. Blacklow, A. L. Kung, D. G. Gilliland, G. L. Verdine, J. E. Bradner, Nature, 2009, 462, 182-188.
45 C. Phillips, L. R. Roberts, M. Schade, R. Bazin, A. Bent, N. L. Davies, R. Moore, A. D. Pannifer, A. R. Pickford, S. H. Prior, C. M. Read, A. Scott, D. G. Brown, B. Xu, S. L. Irving, J. Am. Chem. Soc., 2011, 133, 9696-9699.
46 K. Takada, D. Zhu, G. H. Bird, K. Sukhdeo, J. J. Zhao, M. Mani, M. Lemieux, D. E. Carrasco, J. Ryan, D. Horst, M. Fulciniti, N. C. Munshi, W. Xu, A. L. Kung, R. A. Shivdasani, L. D. Walensky, D. R. Carrasco, Sci. Transl. Med., 2012, 4, 148ra117.
47 L. D. Walensky, A. L. Kung, I. Escher, T. J. Malia, S. Barbuto, R. D. Wright, G. Wagner, G. L. Verdine, S. J. Korsmeyer, Science, 2004, 305, 1466-1470.
48 T. N. Grossmann, J. T. Yeh, B. R. Bowman, Q. Chu, R. E. Moellering, G. L. Verdine, Proc. Natl. Acad. Sci. U.S.A, 2012, 109, 17942-17947.
49 X. Chen, L. Tai, J. Gao, J. Qian, M. Zhang, B. Li, C. Xie, L. Lu, W. Lu, W. Lu, J. Control. Release, 2015, 218, 29-35.
50 H. K. Cui, B. Zhao, Y. Li, Y. Guo, H. Hu, L. Liu, Y. G. Chen, Cell Res., 2013, 23, 581-584.
51 M. Wade, Y. C. Li, G. M. Wahl, Nat. Rev. Cancer, 2013, 13, 83-96.
52 K. H. Khoo, C. S. Verma, D. P. Lane, Nat. Rev. Drug Discov., 2014, 13, 217-236.
53 Q. Zhang, S. X. Zeng, H. Lu, Subcell. Biochem., 2014, 85, 281-319.
54 F. Meric-Bernstam, M. N. Saleh, J. R. Infante, S. Goel, G. S. Falchook, G. Shapiro, K. Y. Chung, R. M. Conry, D. S. Hong, J. S.-Z. Wang, U. Steidl, L. D. Walensky, V. Guerlavais, M. Payton, D. A. Annis, M. Aivado, M. R. Patel, J. Clin. Oncol., 2017, 35, 2505-2505.
55 J. M. Chalker, L. Lercher, N. R. Rose, C. J. Schofield, B. G. Davis, Angew. Chem. Int. Ed. Engl., 2012, 51, 1835-1839.
56 N. Haj-Yahya, H. P. Hemantha, R. Meledin, S. Bondalapati, M. Seenaiah, A. Brik, Org. Lett., 2014, 16, 540-543.
57 N. Azizi, F. Aryanasab, M. R. Saidi, Org. Lett., 2006, 8, 5275-5277.
58 N. Azizi, F. Aryanasab, L. Torkiyan, A. Ziyaei, M. R. Saidi, J. Org. Chem., 2006, 71, 3634-3635.
59 W. M. Valentine, V. Amarnath, D. G. Graham, D. C. Anthony, Chem. Res. Toxicol., 1992, 5, 254-262.
60 C. Li, M. Pazgier, C. Li, W. Yuan, M. Liu, G. Wei, W. Y. Lu, W. Lu, J. Mol. Biol., 2010, 398, 200-213.
61 M. Pazgier, M. Liu, G. Zou, W. Yuan, C. Li, C. Li, J. Li, J. Monbo, D. Zella, S. G. Tarasov, W. Lu, Proc. Natl. Acad. Sci. U.S.A, 2009, 106, 4665-4670.
62 S. B. Kent, Chem. Soc. Rev., 2009, 38, 338-351.
63 G.-M. Fang, Y.-M. Li, F. Shen, Y.-C. Huang, J.-B. Li, Y. Lin, H.-K. Cui, L. Liu, Angew. Chem. Int. Ed. Engl., 2011, 50, 7645-7649.
64 G. M. Fang, J. X. Wang, L. Liu, Angew. Chem. Int. Ed. Engl., 2012, 51, 10347-10350.
65 J. S. Zheng, S. Tang, Y. K. Qi, Z. P. Wang, L. Liu, Nat. Protoc., 2013, 8, 2483-2495.
66 P. H. Kussie, S. Gorina, V. Marechal, B. Elenbaas, J. Moreau, A. J. Levine, N. P. Pavletich, Science, 1996, 274, 948-953.
67 C. Zhan, K. Varney, W. Yuan, L. Zhao, W. Lu, J. Am. Chem. Soc., 2012, 134, 6855-6864.
68 C. Zhan, L. Zhao, X. Wei, X. Wu, X. Chen, W. Yuan, W. Y. Lu, M. Pazgier, W. Lu, J. Med. Chem., 2012, 55, 6237-6241.
69 X. Chen, N. Gohain, C. Zhan, W. Y. Lu, M. Pazgier, W. Lu, Oncogene, 2016, 35, 1919-1925.
70 Z. Nikolovska-Coleska, R. Wang, X. Fang, H. Pan, Y. Tomita, P. Li, P. P. Roller, K. Krajewski, N. G. Saito, J. A. Stuckey, S. Wang, Anal. Biochem., 2004, 332, 261-273.
71 J. Polanowska, I. Krokoszynska, H. Czapinska, W. Watorek, M. Dadlez, J. Otlewski, Biochim. Biophys. Acta, 1998, 1386, 189-198.
72 M. Deponte, Biochim. Biophys. Acta, 2013, 1830, 3217-3266.
73 B. Hu, D. M. Gilkes, J. Chen, Cancer Res., 2007, 67, 8810-8817.
74 L. M. Lau, J. K. Nugent, X. Zhao, M. S. Irwin, Oncogene, 2008, 27, 997-1003.
75 W. M. Ongkeko, X. Q. Wang, W. Y. Siu, A. W. S. Lau, K. Yamashita, A. L. Harris, L. S. Cox, R. Y. C. Poon, Curr. Biol., 1999, 9, 829-832.
76 K. P. Feeley, C. M. Adams, R. Mitra, C. M. Eischen, Cancer Res., 2017, 77, 3823-3833.
77 P. E. Dawson, S. B. Kent, Annu. Rev. Biochem, 2000, 69, 923-960.
78 P. E. Dawson, T. W. Muir, I. Clark-Lewis, S. B. Kent, Science, 1994, 266, 776-779.
79 M. Pazgier, M. Liu, G. Zou, W. Yuan, C. Li, C. Li, J. Li, J. Monbo, D. Zella, S. G. Tarasov, W. Lu, Proc. Natl. Acad. Sci. U.S.A, 2009, 106, 4665-4670.
80 S. B. Kent, Chem. Soc. Rev., 2009, 38, 338-351.
81 X. Chen, L. Tai, J. Gao, J. Qian, M. Zhang, B. Li, C. Xie, L. Lu, W. Lu, W. Lu, J. Control. Release, 2015, 218, 29-35.
82 C. Li, M. Pazgier, C. Li, W. Yuan, M. Liu, G. Wei, W. Y. Lu, W. Lu, J. Mol. Biol., 2010, 398, 200-213.
83 M. Liu, C. Li, M. Pazgier, C. Li, Y. Mao, Y. Lv, B. Gu, G. Wei, W. Yuan, C. Zhan, W. Y. Lu, W. Lu, Proc. Natl. Acad. Sci. U.S.A, 2010, 107, 14321-14326.
84 M. Liu, M. Pazgier, C. Li, W. Yuan, C. Li, W. Lu, Angew. Chem. Int. Ed. Engl., 2010, 49, 3649-3652.
85 C. Zhan, K. Varney, W. Yuan, L. Zhao, W. Lu, J. Am. Chem. Soc., 2012, 134, 6855-6864.
86 C. Zhan, L. Zhao, X. Wei, X. Wu, X. Chen, W. Yuan, W. Y. Lu, M. Pazgier, W. Lu, J. Med. Chem., 2012, 55, 6237-6241.
87 K. Ding, Y. Lu, Z. Nikolovska-Coleska, S. Qiu, Y. Ding, W. Gao, J. Stuckey, K. Krajewski, P. P. Roller, Y. Tomita, D. A. Parrish, J. R. Deschamps, S. Wang, J. Am. Chem. Soc., 2005, 127, 10130-10131.
88 D. Wang, K. Chen, J. L. Kulp Iii, P. S. Arora, J. Am. Chem. Soc., 2006, 128, 9248-9256.
89 Z. Otwinowski, W. Minor, Charles W. Carter, Jr., in Methods in Enzymology, Vol. Volume 276, Academic Press, 1997, pp. 307-326.
80 G. N. Murshudov, A. A. Vagin, E. J. Dodson, Acta Crystallogr D Biol Crystallogr, 1997, 53, 240-255.
91 P. Emsley, K. Cowtan, Acta Crystallogr D Biol Crystallogr, 2004, 60, 2126-2132.

A number of patent and non-patent publications are cited herein in order to describe the state of the art to which this disclosure pertains. The entire disclosure of each of these publications is incorporated by reference herein.

While certain embodiments of the present disclosure have been described and/or exemplified above, various other embodiments will be apparent to those skilled in the art from the foregoing disclosure. The present disclosure is, therefore, not limited to the particular embodiments described and/or exemplified, but is capable of considerable variation and modification without departure from the scope and spirit of the appended claims.

1. A method for preparing a dithiocarbamate stapled peptide comprising:
contacting a peptide comprising a dehydroalanine residue and a lysine residue with carbon disulfide to form a dithiocarbamate linker.

2. The method of claim 1, further comprising contacting a peptide comprising a cysteine residue and a lysine residue with a reagent to convert the cysteine residue into a dehydroalanine residue.

3. The method of claim 2, wherein the cysteine residue and the lysine residue are separated by one or more amino acid residues.

4. The method of claim 2, wherein the reagent comprises a 1,4-dihalobutane group.

5. The method of claim 2, wherein the reagent is selected from 2,5-dibromohexanediamide, 1,4-butanediol dimethanesulfonate, 1,4-dibromobutane, 1,4-diiodobutane, and methyl 2,5-dibromopentanoate.

6. The method of claim 2, wherein the reagent is selected from O-mesitylenesulfonylhydroxylamine, 5,5-dithio-bis-(2-nitrobenzoic acid (Ellman's reagent), and 1,2-bis(bromomethyl)benzene.

7. The method of claim 2, wherein the method further comprises adding the reagent to a buffer solution.

8. The method of claim 7, wherein the buffer solution is at a pH in a range of about 8 to about 9.

9. The method of claim 1, wherein the method further comprises adding the peptide comprising a cysteine residue and a lysine residue to a buffer solution.

10. The method of claim 9, wherein the buffer solution is at a pH in a range of about 2 to about 3.

11. The method of claim 7, wherein the buffer solution comprises guanidine hydrochloride and sodium hydrogen phosphate (Na₂HPO₄).

12. The method of claim 7, wherein the method further comprises adding the buffer solution comprising the peptide comprising a cysteine residue and a lysine residue to the buffer solution comprising the reagent.

13. The method of claim 1, wherein the method further comprises adding the carbon disulfide to a solution comprising one or more alcohols and the peptide comprising a dehydroalanine residue and a lysine residue.

14. The method of claim 13, wherein the alcohol is selected from methanol, ethanol, n-propanol, isopropanol, n-butanol, s-butanol, and t-butanol.

15. (canceled)

16. The method of claim 13, wherein the method further comprises adding a base to the solution of the peptide comprising a dehydroalanine residue and a lysine residue.

17. (canceled)

18. The method of claim 1, wherein the dithiocarbamate stapled peptide comprises a peptide backbone comprising 3 or more amino acid residues.

19. The method of claim 18, wherein the peptide backbone comprises 3 to 20 amino acid residues.

20. (canceled)

21. The method of claim 1, wherein the dehydroalanine residue and the lysine residue are separated by 1 to 8 amino acid residues.

22. (canceled)

23. A stapled peptide comprising a peptide backbone and a staple, wherein:
the peptide backbone comprises three or more amino acid residues;
the staple comprises a dithiocarbamate moiety and is attached to a cysteine residue and a lysine residue; and
the cysteine and lysine residues are separated by one or more amino acid residues.

24.-33. (canceled)

34. The stapled peptide of claim 23, wherein the staple comprises a structure of formula (I):

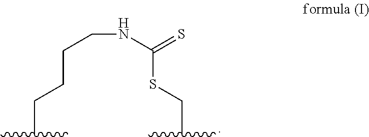 [SMILES: *CCCCNC(=S)SC*]

35. The stapled peptide of claim 23, wherein the stapled peptide is selected from SEQ ID NO: 1 to SEQ ID NO: 11:

| Peptide No. | Peptide Structure |
|---|---|
| SEQ ID NO: 1 | 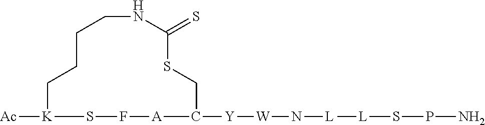 |
| SEQ ID NO: 2 | 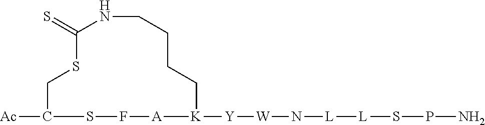 |
| SEQ ID NO: 3 | 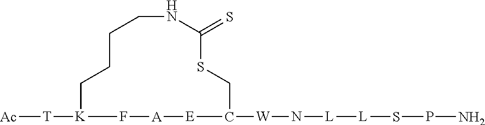 |
| SEQ ID NO: 4 | 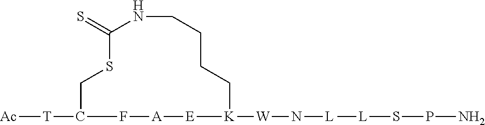 |
| SEQ ID NO: 5 | 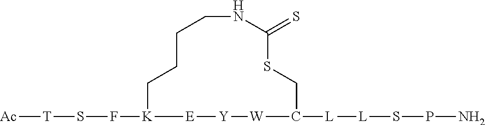 |
| SEQ ID NO: 6 | 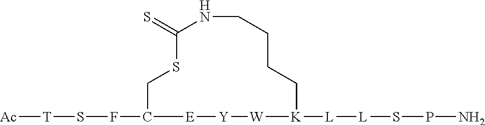 |
| SEQ ID NO: 7 | 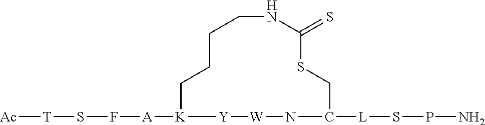 |
| . | |
| SEQ ID NO: 8 | 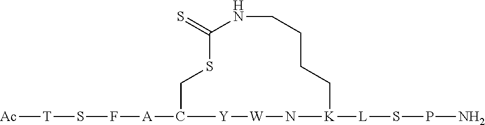 |
| SEQ ID NO: 9 | 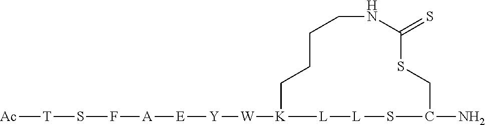 |
| SEQ ID NO: 10 | 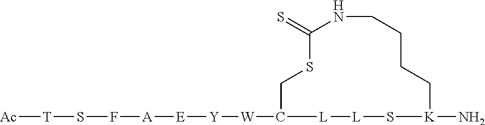 |
| SEQ ID NO: 11 | 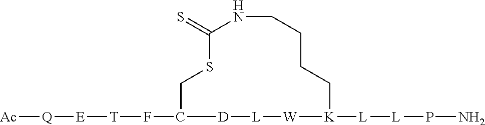 |

36. The stapled peptide of claim 23, wherein the peptide is selected from SEQ ID NO: 5 and SEQ ID NO: 9:

| Peptide No. | Peptide Structure |
|---|---|
| SEQ ID NO: 5 | 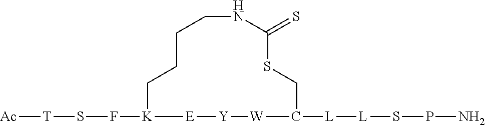 |
| SEQ ID NO: 9 | 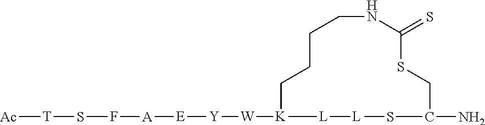 |

37.-44. (canceled)
